(19) United States
(12) Patent Application Publication
(10) Pub. No.: US 2024/0344152 A1
(43) Pub. Date: Oct. 17, 2024

(54) SYSTEMS AND METHODS FOR IDENTIFYING MICROBIAL SIGNATURES

(71) Applicant: Sun Genomics, Inc., San Diego, CA (US)

(72) Inventors: Suneer Jain, San Diego, CA (US); Joann Lieu Phan, San Diego, CA (US); Divya Balachandran Nair, San Diego, CA (US); Thibaut Montagne, San Diego, CA (US)

(21) Appl. No.: 18/574,720

(22) PCT Filed: Jun. 27, 2022
(86) PCT No.: PCT/US2022/035173

§ 371 Date: Dec. 27, 2023

Related U.S. Application Data
(60) Provisional application No. 63/297,638, filed on Jan. 7, 2022.
(60) Provisional application No. 63/237,436, filed on Aug. 26, 2021.
(60) Provisional application No. 63/215,845, filed on Jun. 28, 2021.

(51) Int. Cl. C12Q 1/689; C12Q 1/6851; C12Q 1/6874

(52) U.S. Cl. CPC C12Q 1/689; C12Q 1/6851; C12Q 1/6874; C12Q 2600/118

(57) ABSTRACT

The present disclosure provides systems and methods for identifying microbial signatures from samples that determine discriminant features between healthy control populations and diseased subjects or populations with specific disorders. The microbial signatures are used to diagnose, prognose or determine risk and/or severity of a disease or disorder in a subject, such as autism spectrum disorder or irritable bowel syndrome.

### CROSS-REFERENCE TO RELATED APPLICATIONS

This application claims benefit of priority under 35 U.S.C. § 119 (c) of U.S. Provisional Patent Application Ser. No. 63/297,638, filed Jan. 7, 2022; U.S. Provisional Patent Application Ser. No. 63/237,436, filed Aug. 26, 2021; and U.S. Provisional Patent Application Ser. No. 63/215,845, filed Jun. 28, 2021. The disclosure of the prior applications are considered part of and are incorporated by reference in the disclosure of this application.

### BACKGROUND OF THE INVENTION

### Field of the Invention

The present invention relates generally to the microbiome, and more specifically to systems and methods for determining microbial signatures in a sample that are determinant of discriminant features between healthy control populations and populations with a disease or disorder to diagnose and treat the disease or disorder in a subject.

### Background Information

Early development of the gut microbiota plays an essential role, if not required, for healthy Gastrointestinal (GI) function. About 100 trillion microorganisms live in and on the human body vastly outnumbering the body's approximately 10 trillion human cells. These normally harmless viruses, bacteria and fungi are referred to as commensal or mutualistic organisms. Commensal and mutualistic organisms help keep our bodies healthy in many ways. Together all of the microorganisms living in and on the body-commensal, mutualistic and pathogenic—are referred to as the microbiome and their equilibrium and associated metabolome is closely linked to an individual's health status and vice-versa.

Advances in nucleic acid sequencing has created an opportunity to quickly and accurately identify and profile the microbiome inhabiting the gut and subcutaneous tissue. The optimal flora also interacts with the host immune system in a synergistic way further propagating its health benefits. The associated metabolome of individuals can also be profiled either by a mass-spectrometry based system or using genomics-based metabolome modeling and flux-balance analysis and used to make a healthy metabolome profile. All these methodologies can be used to dissect the complexity of microbial communities.

Improvements in metagenomics (the analysis of more than one organism's DNA within a sample), computational speeds, and software have rapidly advanced our ability to access the microflora of an individual's gut. There is great variability in the human gut microbiome (the community of organisms that make up a human gut microflora) across individuals with some differences associated with host diet, which affect gut microbiome communities at the abundance and functional levels, intake of drugs and antibiotics, interactions with pathogens and parasites and lastly, microbe to microbe contact and engagement with their environment.

The status of an individual's microbiome has been linked to an individual's overall health and can be used in diagnosing disease, as well as treating disease through use of customized probiotics to alter the presence or relative abundances of microbes that make up the microbiome. Previous studies have lacked sample size, sufficient ability to stratify data sets, and sufficient resolution of the data sets when looking at the microbiome (16s vs. WGS) to determine microbial signatures of the microbiome that are indicative of disease.

### SUMMARY OF THE INVENTION

The present invention provides systems and methods for identifying microbial signatures of a microbiome that can be utilized to diagnose, prognose and/or determine risk or severity of a disease or disorder.

Accordingly, in one aspect, the invention provides a method for determining a microbial signature in a microbiome. The method includes:
analyzing microbiomes from a healthy control population, wherein analyzing comprises performing metagenomics analysis and classifying microbial taxa and/or biological pathways;
analyzing microbiomes from a population having a disease or disorder, wherein analyzing comprises performing metagenomics analysis and classifying microbial taxa and/or biological pathways; 2
identifying a microbial signature by comparing the analysis of microbiomes from the healthy control population to the analysis of the microbiomes from the population having the disease or disorder, wherein identifying comprises determining a difference in presence or relative abundances of i) microbial taxa, and/or ii) biological pathways, between the microbiomes from the healthy control population and the microbiomes from the population having the disease or disorder, thereby determining a microbial signature in a microbiome.

In some embodiments, the method further includes using the microbial signature to determine presence, risk or severity of the disease or disorder in a subject.

In some embodiments, the method further includes:
obtaining a sample comprising a microbiome from the subject;
analyzing the microbiome from the subject, wherein analyzing comprises performing metagenomics analysis; and
determining presence and/or relative abundance of the microbial signature in the microbiome from the subject.

In another aspect, the invention provides a method of diagnosing, prognosing and/or determining risk or severity of irritable bowel syndrome (IBS) in a subject. The method includes:
obtaining a sample comprising a microbiome from the subject;
performing metagenomics analysis on the sample;
classifying microbial taxa and/or biological pathways in the microbiome;
determining a microbial signature of the microbiome indicative of IBS based on the classifying; and
diagnosing, prognosing and/or determining risk or severity of IBS in the subject based on the microbial signature, thereby diagnosing, prognosing and/or determining risk or severity IBS in the subject.

In another aspect, the invention provides a method of treating irritable bowel syndrome (IBS) in a subject. The method includes diagnosing the subject as having, or at risk or having, IBS using the method of the invention, and administering the subject a therapeutic to treat IBS in the subject. In a related aspect, the method includes administering the subject having IBS a dietary supplement, such as a probiotic formulation of the invention.

In yet another aspect, the invention provides a method of diagnosing, prognosing and/or determining risk or severity of autism spectrum disorder (ASD) in a subject. The method includes:
obtaining a sample comprising a microbiome from the subject;
performing metagenomics analysis on the sample;
classifying microbial taxa and/or biological pathways in the microbiome;
determining a microbial signature of the microbiome indicative of ASD based on the classifying; and
diagnosing, prognosing and/or determining risk or severity of ASD in the subject based on the microbial signature, thereby diagnosing, prognosing and/or determining risk or severity ASD in the subject.

In still another aspect, the invention provides a method of treating autism spectrum disorder (ASD) in a subject. The method includes diagnosing the subject as having, or at risk or having, ASD using the method of the invention, and administering the subject a therapeutic to treat ASD in the subject. In a related aspect, the method includes administering the subject having ASD a dietary supplement, such as a probiotic formulation of the invention.

In another aspect, the invention provides a probiotic formulation including one or more of Eubacterium rectale and Faecalibacterium prausnitzii, Paraprevotella clara, Prevotella corporis, Roseburia intestinalis, Ruminococcus lactaris or any combination thereof.

In another aspect, the invention provides a dietary supplement that inhibits growth and/or proliferation of one or more of Actinobacteria bacterium, Paracoccidioides brasiliensis, Plasmodium knowlesi, Collectotrichim higginsianum, Xanthomanas vesicatoria, Rhodococcus 852002-51564, Klebsiella MS 92-3, Trypanosoma cruzi and Shigella flexneri.

In another aspect, the invention provides a non-transitory computer readable storage medium encoded with a computer program, the program having instructions that when executed by one or more processors cause the one or more processors to perform operations to perform a method of the present invention.

In another aspect, the invention provides a computing system comprising: a memory; and one or more processors coupled to the memory, the one or more processors configured to perform operations to perform a method of the present invention.

Both the foregoing general description and the following detailed description are exemplary and explanatory only and are intended to provide further explanation of the invention as claimed. Any accompanying drawings are included to provide a further understanding of the invention and are incorporated in and constitute part of this specification, illustrate several embodiments of the invention, and together with the description serve to explain the principles of the invention.

### BRIEF DESCRIPTION OF THE DRAWINGS

FIGS. 1A-1D are graphical representations showing the microbiome profiles of the healthy and IBS cohorts. A) Principal coordinates analysis based on the Bray-Curtis dissimilarity distance matrix of the IBS and healthy microbiomes. B) Boxplot of the microbiome distributions along the PCO1 axis. An unpaired t-test was computed. C) A random forest was employed to differentiate microbes between healthy and IBS subtypes. The density of microbes selected from random forest correspond to the sample distribution along PCO1 axis. D) Alpha diversity between healthy and each IBS subtype cohort. The Shannon index, species richness, and evenness were calculated. Unpaired t-tests were conducted, and p-values were adjusted with Benjamin-Hochberg false discovery rate (FDR) for multiple comparisons. * p<0.05, ** p<0.01, *** p<0.001, **** p<0.0001.

FIGS. 2A-2B are graphical representations showing microbes and pathways that differentiate healthy and IBS cohorts. A) Relative abundances of the microbes associated with healthy and IBS populations. A random forest was used to determine microbes that contribute to differentiating healthy and IBS. The relative abundances of the microbes were plotted for healthy and each IBS subtype. T-test were calculated. P values were adjusted for multiple comparisons testing by false discovery rate corrections. Non-significant comparisons were omitted. B) Functional pathways associated with healthy and IBS gut microbiomes. Multivariate linear association testing with Maaslin2 was used to determine pathways associated with IBS relative to the healthy control population. Values indicate the beta coefficient from linear association testing. Pathways listed were filtered based on q value<0.1 and beta coefficients>0.2 or <−0.2. * p<0.05, ** p<0.01, *** p<0.001, **** p<0.0001.

FIGS. 3A-3D are graphical representations showing longitudinal microbiome diversity and relative abundances of probiotics in subjects with IBS. A) Shannon index of the microbiome composition from subjects with timepoints 1-3. B) Bray-Curtis similarity of timepoints within each individual. Each timepoint is compared to each subsequent timepoint. C) The overall microbiome score across timepoints 1-3. Wilcoxon tests were computed with FDR adjusted p values. D) Relative abundances of probiotic species in subjects across 3 timepoints. T-tests were computed with FDR adjusted p values. * p value<0.05, ** p value<0.01, *** p value<0.001.

FIG. 4 is a graphical plot showing principal coordinates analysis of microbiome composition from healthy and ASD populations. Shape indicates the ASD and healthy control populations. Healthy control microbiome samples cluster more closely together than the ASD microbiome samples. The larger spread among the ASD microbiome samples indicate greater differences within the ASD microbiome sample cohort and between the healthy cohort. Different factors may contribute to the changes in microbiome composition.

FIG. 5 is a series of graphical plots showing alpha diversity across ASD and NT cohorts at time point 1 (T1).

FIG. 6 is a series of graphical plots showing alpha diversity of ASD across T1 and T2 (paired) using the ASD cohort only.

FIG. 7 is a graphical plot showing alpha diversity of ASD across T1 and T2 (paired) with ASD compared to NT at T1.

FIG. 8 shows a Random Forest analysis including microbial variables of importance and identifying microbial signatures that distinguish Healthy and ASD subjects. Mean decrease Gini values are plotted for each of the top 50 microbes.

FIG. 9 is a series of graphical plots of relative abundances of microbes in ASD and neurotypical (NT) cohorts. Relative abundances of the microbes shown were higher in abundance in ASD compared to the NT cohort. Microbes were selected from the random forest algorithm and were significantly different between the two populations.

FIG. 10 is a series of graphical plots of relative abundances of microbes in ASD and NT cohorts. Relative abundances of the microbes shown were lower in abundance in ASD compared to the NT cohort. Microbes were selected from the random forest algorithm and were significantly different between the two populations.

FIG. 11 is a graphical plot showing microbial variables of importance from a random forest used to distinguish abundance of biological pathways of ASD and NT baseline cohorts. The mean decrease Gini values are plotted for each of the top 50 pathways.

FIG. 12 is a series of graphical plots showing relative abundances of biological pathways in ASD and NT cohorts selected from the random forest algorithm. Relative abundances of the pathways shown were higher in abundance in ASD compared to the NT cohort. Pathways were selected from the random forest algorithm and were significantly different between the two populations.

FIG. 13 is a series of graphical plots showing relative abundances of biological pathways in ASD and NT cohorts selected from the random forest algorithm. Relative abundances of the pathways shown were lower in abundance in ASD compared to the NT cohort. Pathways were selected from the random forest algorithm and were significant different between the two populations.

FIG. 14 is a graphical plot showing microbial variables of importance from a random forest used to distinguish gene families of ASD and NT cohorts. Mean decrease Gini values are plotted for each of the top 50 gene families.

FIG. 15 is a series of graphical plots showing relative abundances of gene families in ASD and NT cohorts. Relative abundances of the gene families shown were higher in abundance in ASD compared to the NT cohort. Gene families were selected from the random forest algorithm and were significant different between the two populations.

FIG. 16 is a series of graphical plots showing relative abundances of gene families in ASD and NT cohorts. Relative abundances of the gene families shown were lower in abundance in ASD compared to the NT cohort. Gene families were selected from the random forest algorithm and were significant different between the two populations.

FIG. 17 is a graphical plot showing a significant biological pathway between ASD and NT cohorts.

FIG. 18 is a graphical plot showing a significant biological pathway between ASD and NT cohorts.

FIG. 19 is a graphical plot showing a significant biological pathway between ASD and NT cohorts.

FIG. 20 is a graphical plot showing a significant biological pathway between ASD and NT cohorts.

FIG. 21 is a graphical plot showing a significant biological pathway between ASD and NT cohorts.

FIG. 22 shows PGIA survey questions at T1.

FIG. 23 shows PGIA survey questions at T2.

FIG. 24 is a series of histograms representing results of parental global impressions of autism (PGIA) survey questions at timepoint 2, which is approximately 3 months after synbiotic usage. Each of the panels represent a phenotype in which the parent assesses improvement or worsening of symptoms observed in their child.

FIG. 25 is a series of histograms representing PGIA survey questions in longitudinal samples. Grey scale shades represent different timepoints.

FIG. 26 is a graphical plot representing PGIA T2+ survey score. The number above each boxplot indicates the number of participants that are evaluated at each timepoint.

FIG. 27 is a graphical plot representing PGIA T2+ scores at timepoint 2 and 3 for paired samples. There are 32 subjects with paired samples. There is a significant increase in the PGIA T2+ score, indicating an overall improvement in phenotypes assessed by this survey.

FIG. 28 is a series of graphical plots showing the Pearson correlations between the Shannon Index, species richness, and evenness with the nutritional assessment survey scores at baseline.

FIG. 29 is a series of graphical plots showing the Pearson correlations between the Shannon Index, species richness, and evenness with the PGIA survey scores at baseline.

FIG. 30 is a series of graphical plots showing the Pearson correlations between the Shannon Index, species richness, and evenness with the SRS2 survey scores at baseline.

FIG. 31 is a series of graphical plots showing the Pearson correlations between the Shannon Index, species richness, and evenness with the SCARED phenotypic survey scores at baseline. Results indicate the SCARED score survey is inversely correlated to microbial evenness and close to significant with the Shannon diversity index.

FIG. 32 is a series of graphical plots showing the Pearson correlations between alpha diversity and the gastrointestinal symptom rating scale (GSRS).

FIG. 33 is a graphical plot showing Pearson correlation between nutritional assessment and PGIA survey. There is a significant correlation between nutritional assessment and PGIA (p=0.025, R=−0.19).

FIG. 34 is a graphical plot showing longitudinal social responsiveness scale scores. Normalized T scores from participants with both timepoints were assessed to identify changes in autism severity. Sample 1 is from baseline while Sample 2 was taken after approximately 3 months on the personalized synbiotic. Results indicate there were no significant differences between timepoints on average.

FIG. 35 is a graphical plot showing SRS2 survey of subjects who showed improvement or worsening of symptoms. Of the participants who improved, there was a significant decrease in the T score while there was no significant increase in the T score in subjects with negative response or no change across Samples 1 and 2.

FIG. 36 is a series of graphical plots showing GSRS scores. Scores from participants with both timepoints were assessed to identify changes in gastrointestinal symptoms of abdominal pain, reflux syndrome, diarrhea, ingestion syndrome, constipation, and overall gastrointestinal health. Sample 1 is from baseline while Sample 2 was taken after approximately 3 months on the personalized synbiotic. Results indicate while there were no significant changes in the individual symptoms on average, there was a significant decrease in symptom severity in the total score. The total score takes into consideration all symptoms assessed by the survey.

FIG. 37 is a series of graphical plots showing GSRS scoring for each gastrointestinal phenotype as well as the overall total score. The value above each boxplot indicates the number of surveys or participants that have taken the survey at each timepoint.

FIG. 38 is a graphical plot showing the top 20 microbes from pilot whale gut microbiomes (Healthy vs Sick whale).

### DETAILED DESCRIPTION OF THE INVENTION

The present disclosure provides systems and methods for identifying microbial signatures from samples that determine discriminant genomic features between a healthy control population and diseased population. Metagenomic sequencing captures all genomic content of the sample, providing insight into microbial identity and protein coding and non-coding genetic materials. Microbes include eukaryotes and prokaryotes, including bacteria, parasites, archaea, fungi, viruses, phage, and others. As described herein, genomic content that infers functional potential was computationally assessed to find the underlying differences in metabolisms between microbiomes from healthy and diseased populations. The microbial signatures that were identified by comparing differences in microbiomes from healthy and diseased populations are used to diagnose, prognose and/or determine risk or severity of a disease or disorder in a subject, such as IBS and ASD.

Before the present compositions and methods are described, it is to be understood that this invention is not limited to the particular methods and systems described, as such methods and systems may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one aspect, the invention provides a method for determining a microbial signature in a microbiome. The method includes:
analyzing microbiomes from a healthy control population, wherein analyzing comprises performing metagenomics analysis and classifying microbial taxa and/or biological pathways;
analyzing microbiomes from a population having a disease or disorder, wherein analyzing comprises performing metagenomics analysis and classifying microbial taxa and/or biological pathways; and
identifying a microbial signature by comparing the analysis of microbiomes from the healthy control population to the analysis of the microbiomes from the population having the disease or disorder, wherein identifying comprises determining a difference in presence or relative abundances of i) microbial taxa, and/or ii) biological pathways, between the microbiomes from the healthy control population and the microbiomes from the population having the disease or disorder, thereby determining a microbial signature in a microbiome.

In various embodiments, microbiome analysis utilizes a universal method for extracting nucleic acid molecules from a diverse population of one or more types of microbes in a sample. In various embodiments, the types of microbes include, but are not limited to, gram-positive bacteria, gram-positive bacterial spores, gram-negative bacteria, archaea, protozoa, helminths, algae, fungi, fungal spores, viruses, viroids, bacteriophages, and rotifers. In some aspects, the diverse population is a plurality of different microbes of the same type, e.g., gram-positive bacteria. In some aspects, the diverse population is a plurality of different types of microbes, e.g., bacteria (gram-positive bacteria, gram-positive bacterial spores and/or gram-negative), fungi, viruses, and bacteriophages.

As used herein, the term “microbiome” refers to microorganisms, including, but not limited to bacteria, phages, viruses, and fungi, archaea, protozoa, amoeba, or helminths that inhabit the gut of a subject.

As used herein, the terms “microbial”, “microbe”, and “microorganism” refer to any microscopic organism including prokaryotes or eukaryotes, spores, bacterium, archeaebacterium, fungus, virus, or protist, unicellular or multicellular.

As used herein, the term “microbial signature” refers to presence, absence or relative abundace of a genetic signature indicative of a disease or disorder. Such genetic signatures may be associated with presence, absence or relative abundance of different types of microbial taxa in a microbiome, or genetic signatures may be associated with presence, absence or relative abundance of a biological pathway in the microbiome.

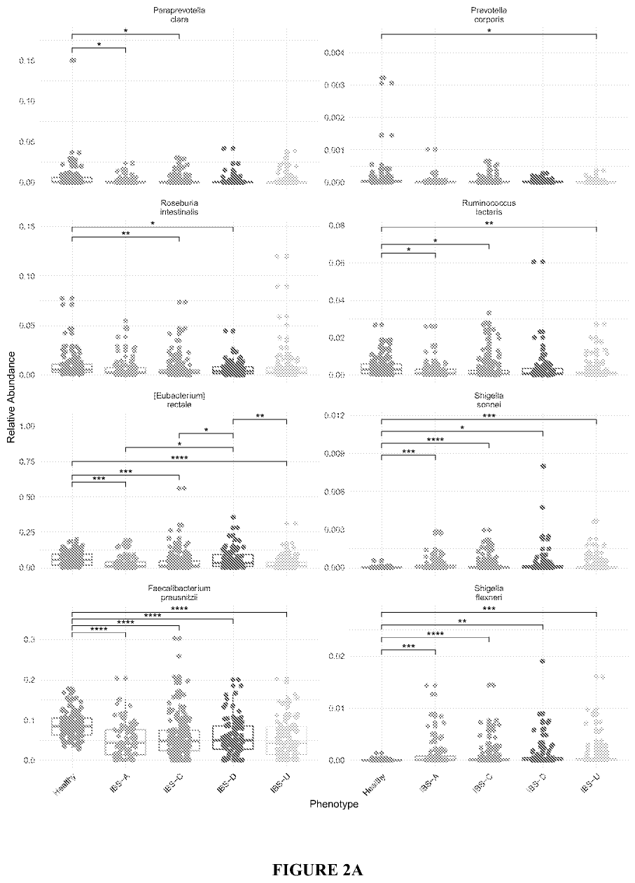

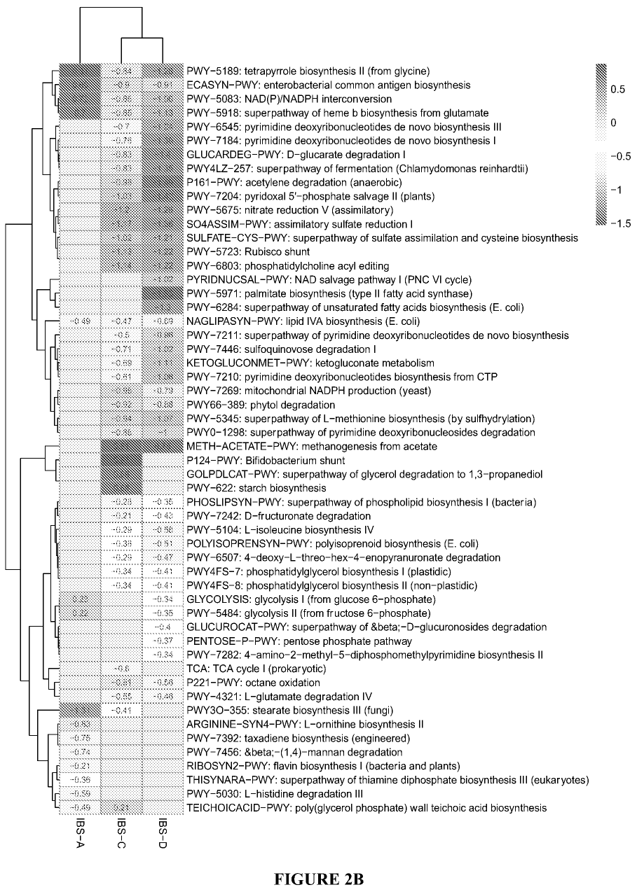

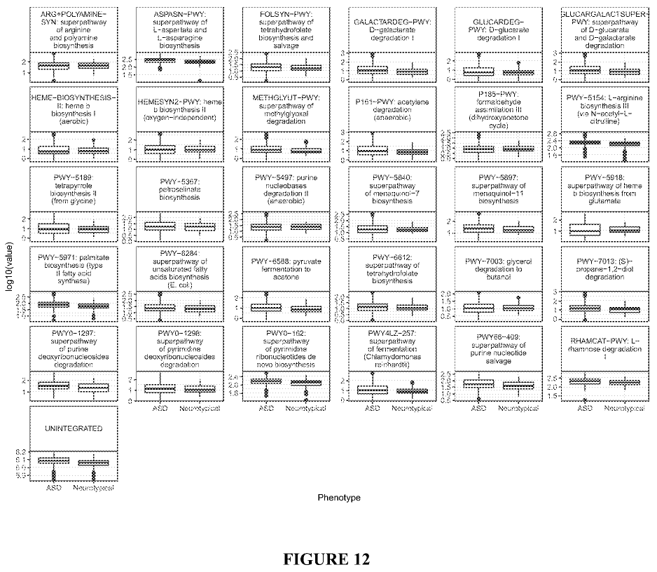

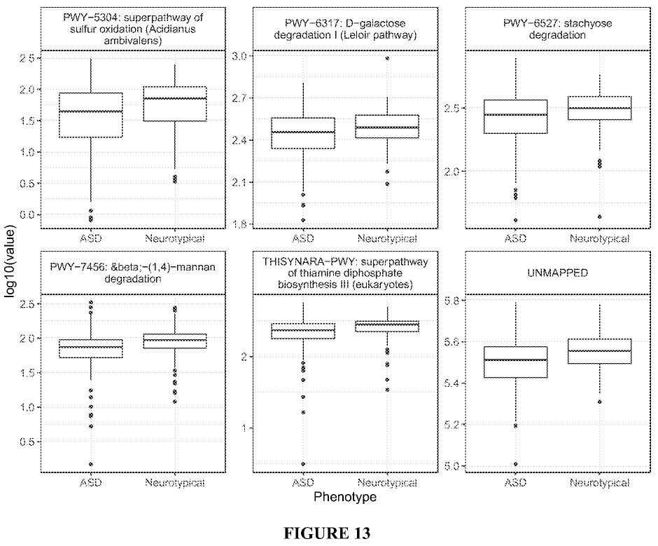

As used herein, the term “biological pathway” refers to any pathway any pathway in a biological system that includes a series of interactions among molecules in a cell that leads to a certain product or a change in a cell. Illustrative biological pathways for use with the invention include those listed in Table 3 and FIGS. 2B and 11-13.

As used herein, the term “subject” or “patient” includes humans and non-human animals. The term “non-human animal” includes all vertebrates, e.g., mammals and non-mammals, such as non-human primates, whales, elephants, horses, sheep, dogs, cats, cows, pigs, chickens, and other veterinary subjects and test animals.

As used herein, the terms “polynucleotide”, “nucleic acid” and “oligonucleotide” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cfDNA and cell-free RNA (cfRNA), nucleic acid probes, and primers. A polynucleotide may include one or more modified nucleotides, such as methylated nucleotides and nucleotide analogs.

In various embodiments, metagenomics analysis includes analysis of any type and length of nucleic acid. This nucleic acid can be of any length, as short as oligos of about 5 bp to as long a megabase or even longer. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule.

Because different types of microbes have different compositions and mechanisms to protect their own genetic material it is often difficult to extract the genetic material from one type of microbe without compromising the ability to also extract the genetic material of another type of microbe in the same biological sample. The present invention, however, utlizes techniques that allow the extraction of genetic material from different types of microbes in a sample without sacrificing the amount of genetic material that can be obtained from one type of microbe by extracting the genetic material of another type of microbe in the same sample. As will be appreciated, this is particularly advantageous for extraction of nucleic acid from a diverse population of microbes in performing metagenomics analysis of a microbiome.

In various embodiments, the methodology of the present invention includes extracting and analyzing nucleic acids present in a biological sample obtained from a subject to perform microbiome analysis. The methodology also includes extracting and analyzing nucleic acids present in biological samples obtained from a population of subjects to perform microbiome analysis, e.g., a healthy contol population and a population having a disease or disorder. The methodology also includes extracting and analyzing nucleic acids present in a biological sample obtained from a subject to detect a microbial signature that is indicative of a disease or disorder. The methodology also includes extracting and analyzing nucleic acids present in biological samples obtained from a population of subjects, e.g., a healthy contol population and a population having a disease or disorder, to detect a microbial signature that is indicative of a disease or disorder.

In various embodiments, the metagenomics analysis is perfomed using a biological sample that includes microbes. In some embodiments, a sample is a gut or fecal sample obtained by non-invasive or invasive techniques such as biopsy of a subject. In one aspect, the term “sample” refers to any preparation derived from fecal matter or gut tissue of a subject. For example, a sample of material obtained using the non-invasive method described herein can be used to isolate nucleic acid molecules for the methods of the present invention. In some embodiments, biological secretions are obtained from the digestive tract. Solid samples may be liquefied or mixed with a solution, and then genetic material may be extracted in accordance with any nucleic acid extraction protocols known in the art. In some embodiments, the extracted genetic material may be subjected to further processing and analysis, such as purification, amplification, and sequencing. In some embodiments, the extracted genetic material is subjected to metagenomics analysis to, for example, identify the one or more types of organisms in the sample from which the genetic material was extracted.

In some embodiments, the extracted genetic material is subjected to metagenomics analysis to, for example, identify the one or more types of microbes in the sample from which the genetic material was extracted for microbiome analysis. In additional embodiments, full whole genome shotgun sequencing can be performed on prepared extracted nucleic acid material from human fecal samples. Preparations include nucleic acid clean up reactions to remove organic solvents, impurities, salts, phenols, and other process inhibiting contaminants. Additional preparations include nucleic acid library prep from each sample where the gDNA is subject to modifications and/or amplifications to prep the sample for sequencing on a sequencing platform such as massively parallel sequencing by synthesis, nanopore, long read, and/or CMOS electronic, sequencing methods. In some aspects, nucleic acid is extracted and processed for microbiome analysis as described in International Patent Application No. PCT/US2019/058224, the content of which is incorporated by reference in its entirety.

In the various aspects discussed herein, processing steps may include, RNA or DNA clean-up, fragmentation, separation, or digestion; library or nucleic acid preparation for downstream applications, such as PCR, qPCR, digital PCR, or sequencing; preprocessing for bioinformatic QC, filtering, alignment, or data segregation; metagenomics or human genomic bioinformatics pipeline for microbial species taxonomic assignment; and other organism alignment, identification, and variant interpretation.

In certain aspects, the method of the present invention uses stool samples obtained from a subject for DNA extraction and microbiome analysis. In some aspects, the extracted genetic material is subjected to further processing and analysis, such as purification, amplification and sequencing. In various aspects, the method further includes subjecting the extracted genetic material to metagenomics analysis to, for example, to identify the one or more types of organisms in the sample from which the genetic material was extracted.

In some aspects, the database that the metagenomics analysis utilizes has been customized for a specific purpose of identifying and taxonomically assigning, within the appropriate phylogeny, the nucleic acids with relative abundances of microbial taxa to determine a microbial signature. In some embodiments, the database that the metagenomics analysis utilizes has been customized for a specific purpose of identifying absence, presence or relative abundance of genetic regions associated with a particular biological pathway to determine a microbial signature. Such regions may be non-coding or coding regions, such as genes. In some embodiments, an additional data table or database may be used as a lookup of the relative abundances of microbial taxa content of a sample or absence, presence or relative abundances of genetic regions associated with a particular biological pathway present in a sample.

In some aspects, extracted and purified genetic material is prepared for sequencing using Illumina index adaptors and checked for sizing and quantity. A range from 1000 or greater reads of sequencing for short insert methods can be used for this method. Large insert methods such as Pac Bio™, Nanopore™, or other next generation sequencing methods can use <1000 sequencing reads. Bioinformatics quality filtering was performed before taxonomy assignment. Quality trimming of raw sequencing files may include removal of sequencing adaptors or indexes; trimming 3′ or 5′ end of reads based on quality scores (Q20>), basepairs of end, or signal intensity; removal of reads based on quality scores, GC content, or non-aligned basepairs; removal of overlapping reads at set number of base pairs. Alignment of processed sequencing files was done using a custom microbial genome database consisting of sequences from Refseq™, Greengeens™, HMP™, NCBI™, PATRIC™, or other public/private data repositories or in-house data sets. This database may be used as full genome alignment scaffold, k-mer fragment alignment, or other schemes practiced in the art of metagenomics and bioinformatics. Based off the number of sequencing reads/fragments that match the database genomes we assign a taxonomic identity that is common or unique to the organism. This identifier can be a barcode, nucleotide sequence, or some other computational tag that will associate the matching sequencing read to an organism or strain within a taxonomic group. Some identifiers will be of higher order and would identify domain, kingdom, phylum, class, order, family, or genus of the organism.

In various embodiments, the present invention is able to identify and/or classify a microorganism at the lowest order of strain within a species.

In some aspects, sequencing of the nucleic acid from the sample is performed using whole genome sequencing (WGS) or rapid WGS (rWGS). In some aspects, targeted sequencing is performed and may be either DNA or RNA sequencing. The targeted sequencing may be to a subset of the whole genome. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain aspects, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (e.g, singleplex sequencing) or DNA from multiple samples can be pooled and sequenced as indexed genomic molecules (e.g, multiplex sequencing) on a single sequencing run, to generate up to several hundred million reads of DNA sequences. Commercially available platforms include, e.g, platforms for sequencing-by-synthesis, ion semiconductor sequencing, pyrosequencing, reversible dye terminator sequencing, sequencing by ligation, single-molecule sequencing, sequencing by hybridization, and nanopore sequencing. In some aspects, the methodology of the disclosure utilizes systems such as those provided by Illumina, Inc, (HiSeq™ X10, HiSeq™ 1000, HiSeq™ 2000, HiSeq™ 2500, HiSeq™ 4000, NovaSeq™ 6000, Genome Analyzers™, MiSeq™ systems), Applied Biosystems Life Technologies (ABI PRISM™ Sequence detection systems, SOLID™ System, Ion PGM™ Sequencer, ion Proton™ Sequencer).

In various embodiments, the methodology of the invention utilizes statistical analysis, for example, to identify a microbial signature by determining presence, absence or relative abundances of microbial taxa and/or biological pathways. Statistical analysis can include any statistical method useful for analyzing and comparing data sets, such as univariate, multivariate, and machine learning approaches to profile community wide microbial features. Permutational multivariate analysis of variance may be used to calculate the percentage of variation explained by disease status. It will be appreciated that any statistical methods known in the art may be utilized for assessment of similarity, divergence, uniqueness, or distance. In some embodiments, statistical analysis includes principal coordinate or a component analysis and/or a clustering model, such as random forest or permutated random forest.

It will be understood that the present methodology may be used to determine a microbial signatures that are indicative of any number of diseases or disorders in which a microbial signature is determined between microbiomes of a healthy control population and a diseased population. By way of illustration, a disease or disorder may include irritable bowel syndrome (IBS), autism spectrum disorder (ASD), arthritis, obesity, dysbiosis, Crohn's disease, mood disorder, chronic fatigue, infection, necrosis, inflammation, autoimmune, hemorrhage, weight loss, metabolic disorder, diabetes 1 or 2, rheumatoid arthritis, cancer, and cardiovascular disorder. The Examples set forth herein, exemplify use of the methodology of the invention to diagnose IBS and ASD.

Accordingly, in another aspect, the invention provides a method of diagnosing, prognosing and/or determining risk or severity of irritable bowel syndrome (IBS) in a subject. The method includes:
obtaining a sample comprising a microbiome from the subject;
performing metagenomics analysis on the sample;
classifying microbial taxa and/or biological pathways in the microbiome;
determining a microbial signature of the microbiome indicative of IBS based on the classifying; and
diagnosing, prognosing and/or determining risk or severity of IBS in the subject based on the microbial signature, thereby diagnosing, prognosing and/or determining risk or severity IBS in the subject.

In various embodiments, the microbial signature indicative of IBS is presence, absence or relative abundances of microbial taxa including one or more of Paraprevotella clara, Prevotella corporis, Roseburia intestinalis, Ruminococcus lactaris, Eubacterium rectale, Shigella sonnei, Faecalibacterium prausnitzii, and Shigella flexneri. In some embodiments, the microbial signature is an increase in relative abundances of Enterobacterales species, such as Shigella. In some embodiments, the microbial signature is a decrease in relative abundances of one or more of Eubacterium rectale, Faecalibacterium prausnitzii, Paraprevotella clara, Prevotella corporis, Roseburia intestinalis or Ruminococcus lactaris.

In embodiments, the microbial signature indicative of IBS is presence, absence or relative abundances of microbial genus including one or more of Bacteroides, Faccalibacterium, Parabacteroides, Roseburia, Bifidobacterium, Enterobacteriaceae, Clostridium, Streptococcus, Anacrostipes, Lactbacillus, Alistipes and Pseudomonas.

In some embodiments, the microbial signature is indicative of Crohn's disease in includes increased presence of one or more of Faccalibacterium, Roseburia, a Eubacterium hallii group, Butyricicoccus, Anacrostipes, Flavonifractor, Odoribacter, Butyricimonas, Butyrivibrio and Coprococcus.

In various embodiments, the microbial signature indicative of IBS is presence, absence or relative abundances of biological pathways including one or more of those set forth in FIG. 2 or Table 3. For example, the microbial signature may be an increase in relative abundances of biological pathways associated with one or more of tetrapyrrole biosynthesis from glycine, enterobacterial common antigen biosynthesis, NADP/NADPH interconversion, super pathway of heme b biosynthesis from glutamate, methanogenesis from acetate, Bifidobacterium shunt, super pathway of glycerol degradation to 1,3-propanediol, and starch biosynthesis. In addition, the microbial signature may be a decrease in relative abundances of biological pathways associated with amino acid and ribonucleotide biosynthesis, polysaccharide degradation, and fermentation.

In another aspect, the invention provides a method of diagnosing, prognosing and/or determining risk or severity of ASD in a subject. The method includes:
obtaining a sample comprising a microbiome from the subject;
performing metagenomics analysis on the sample;
classifying microbial taxa and/or biological pathways in the microbiome;
determining a microbial signature of the microbiome indicative of ASD based on the classifying; and
diagnosing, prognosing and/or determining risk or severity of ASD in the subject based on the microbial signature, thereby diagnosing, prognosing and/or determining risk or severity ASD in the subject.

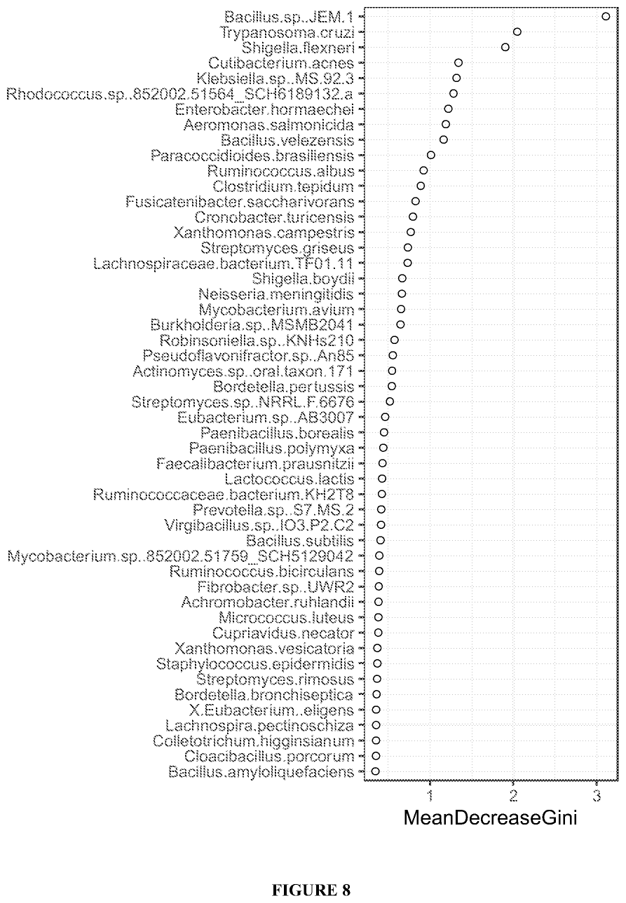

In various embodiments, the microbial signature indicative of ASD is presence, absence or relative abundances of microbial taxa including one or more of Actinobacteria, Paracoccidioides, Plasmodium, Colletotrichum, Xanthomonas, Rhodococcus, Klebsiella, Trypanosoma, Shigella species, and a microbe of a genus listed in FIG. 8. In some embodiments, the microbial signature indicative of ASD is presence, absence or relative abundances of one or more of Actinobacteria bacterium, Paracoccidioides brasiliensis, Plasmodium knowlesi, Collectotrichim higginsianum, Xanthomanas vesicatoria, Rhodococcus 852002-51564, Klebsiella MS 92-3, Trypanosoma cruzi, Bacillus sp. JEM-1, and Shigella flexneri. In some embodiments, the microbial signature is an increase in relative abundances of one or more of Actinobacteria bacterium, Paracoccidioides brasiliensis, Plasmodium knowlesi, Collectotrichim higginsianum, Xanthomanas vesicatoria, Rhodococcus 852002-51564, Klebsiella MS 92-3, Trypanosoma cruzi, and Shigella flexneri. In some embodiments, the microbial signature is an increase in relative abundances of one or more of Actinobacteria, Paracoccidioides, Plasmodium, Colletotrichum, Xanthomonas, Rhodococcus, Klebsiella, Trypanosoma and Shigella species.

In some embodiments, the microbial signature is an increase in relative abundances of one or more of Bacillus.sp.JEM.1, Trypanosoma.cruzi, Shigella.flexneri, Cutibacterium.acnes, Klebsiella.sp.MS.92.3, Rhodococcus.sp.852002.51564_SCH6189132.a, Enterobacter.hormaechei, Aeromonas.salmonicida, Bacillus.velezensis, Paracoccidioides.brasiliensis, Ruminococcus.albus, Clostridium.tepidum, Fusicatenibacter.saccharivorans, Cronobacter.turicensis, Xanthomonas.campestris, Streptomyces.griseus, Lachnospiraceae.bacterium.TF01.11, Shigella.boydii, Neisseria.meningitidis, Mycobacterium.avium, Burkholderia.sp.MSMB2041, Robinsoniella.sp.KNHs210, Pseudoflavonifractor.sp.An85, Actinomyces.sp.oral.taxon.171, Bordetella.pertussis, Streptomyces.sp.NRRL.F.6676, Eubacterium.sp.AB3007, Paenibacillus.borealis, Paenibacillus.polymyxa, Faecalibacterium.prausnitzii, Lactococcus.lactis, Ruminococcaceae.bacterium.KH2T8, Prevotella.sp.S7.MS.2, Virgibacillus.sp.103.P2.C2, Bacillus.subtilis, Mycobacterium.sp.852002.51759_SCH5129042, Ruminococcus.bicirculans, Fibrobacter.sp.UWR2, Achromobacter.ruhlandii, Micrococcus.luteus, Cupriavidus.necator, Xanthomonas.vesicatoria, Staphylococcus.epidermidis, Streptomyces.rimosus, Bordetella.bronchiseptica, Eubacterium.eligens, Lachnospira.pectinoschiza, Colletotrichum.higginsianum, Cloacibacillus.porcorum, and Bacillus.amyloliquefaciens.

In some embodiments, the microbial signature is a decrease in relative abundances of one or more of Bacillus.sp.JEM.1, Trypanosoma.cruzi, Shigella.flexneri, Cutibacterium.acnes, Klebsiella.sp.MS.92.3, Rhodococcus.sp.852002.51564_SCH6189132.a, Enterobacter.hormaechei, Aeromonas.salmonicida, Bacillus.velezensis, Paracoccidioides.brasiliensis, Ruminococcus.albus, Clostridium.tepidum, Fusicatenibacter.saccharivorans, Cronobacter.turicensis, Xanthomonas.campestris, Streptomyces.griseus, Lachnospiraceae.bacterium.TF01.11, Shigella.boydii, Neisseria.meningitidis, Mycobacterium.avium, Burkholderia.sp.MSMB2041, Robinsoniella.sp.KNHs210, Pseudoflavonifractor.sp.An85, Actinomyces.sp.oral.taxon.171, Bordetella.pertussis, Streptomyces.sp.NRRL.F.6676, Eubacterium.sp.AB3007, Paenibacillus.borealis, Paenibacillus.polymyxa, Faecalibacterium.prausnitzii, Lactococcus.lactis, Ruminococcaceae.bacterium.KH2T8, Prevotella.sp.S7.MS.2, Virgibacillus.sp.103.P2.C2, Bacillus.subtilis, Mycobacterium.sp.852002.51759_SCH5129042, Ruminococcus.bicirculans, Fibrobacter.sp.UWR2, Achromobacter.ruhlandii, Micrococcus.luteus, Cupriavidus.necator, Xanthomonas.vesicatoria, Staphylococcus.epidermidis, Streptomyces.rimosus, Bordetella.bronchiseptica, Eubacterium.eligens, Lachnospira.pectinoschiza, Colletotrichum.higginsianum, Cloacibacillus.porcorum, and Bacillus.amyloliquefaciens.

In some embodiments, the microbial signature is an increase in relative abundances of Bacillus.sp.JEM.1, Trypanosoma.cruzi and/or Shigella.flexneri.

In some embodiments, the microbial signature is a decrease in relative abundances of Bacillus.sp.JEM.1, Trypanosoma.cruzi and/or Shigella.flexneri.

In various embodiments, the microbial signature indicative of ASD is presence, absence or relative abundances of biological pathways including one or more of those set forth in FIG. 11.

In various embodiments, the microbial signature indicative of ASD is presence, absence or relative abundances of biological pathways including one or more of PWY0-1298: superpathway of pyrimidine deoxyribonucleosides degradation, PWY-7013: (S)-propane-1,2-diol degradation, PWY-7456: &beta;-(1,4)-mannan degradation, METHGLYUT-PWY: superpathway of methylglyoxal degradation, GLUCARDEG-PWY: D-glucarate degradation I, PWY-6612: superpathway of tetrahydrofolate biosynthesis, PWY-5840: superpathway of menaquinol-7 biosynthesis, P161-PWY: acetylene degradation (anaerobic), FOLSYN-PWY: superpathway of tetrahydrofolate biosynthesis and salvage, ASPASN-PWY: superpathway of L-aspartate and L-asparagine biosynthesis, PWY-5971: palmitate biosynthesis (type II fatty acid synthase), PWY0-1297: superpathway of purine deoxyribonucleosides degradation, PWY-6588: pyruvate fermentation to acetone, GALACTARDEG-PWY: D-galactarate degradation I, PWY-6305: superpathway of putrescine biosynthesis, PWY4LZ-257: superpathway of fermentation (Chlamydomonas reinhardtii), GLUCARGALACTSUPER-PWY: superpathway of D-glucarate and D-galactarate degradation, PWY66-409: superpathway of purine nucleotide salvage, PWY-5154: L-arginine biosynthesis III (via N-acetyl-L-citrulline), PWY-5659: GDP-mannose biosynthesis, THISYNARA-PWY: superpathway of thiamine diphosphate biosynthesis III (cukaryotes), PWY-7003: glycerol degradation to butanol, CENTFERM-PWY: pyruvate fermentation to butanoate, PWY-5189: tetrapyrrole biosynthesis II (from glycine), PWY-2942: L-lysine biosynthesis III, HSERMETANA-PWY: L-methionine biosynthesis III, HEMESYN2-PWY: heme b biosynthesis II (oxygen-independent), RHAMCAT-PWY: L-rhamnose degradation I, GLUCUROCAT-PWY: superpathway of &beta;-D-glucuronosides degradation, PWY-6527: stachyose degradation, PWY0-162: superpathway of pyrimidine ribonucleotides de novo biosynthesis, PWY-5497: purine nucleobases degradation II (anaerobic), PWY-6284: superpathway of unsaturated fatty acids biosynthesis (E. coli), PWY-6317: D-galactose degradation I (Leloir pathway), P185-PWY: formaldehyde assimilation III (dihydroxyacetone cycle), PWY-6470: peptidoglycan biosynthesis V (&beta;-lactam resistance), PWY-7242: D-fructuronate degradation, PWY-5304: superpathway of sulfur oxidation (Acidianus ambivalens), ARG+POLYAMINE-SYN: superpathway of arginine and polyamine biosynthesis, PWY-5897: superpathway of menaquinol-11 biosynthesis, PWY-7357: thiamine phosphate formation from pyrithiamine and oxythiamine (yeast), GLYCOGENSYNTH-PWY: glycogen biosynthesis I (from ADP-D-Glucose), PWY-5918: superpathway of heme b biosynthesis from glutamate, PWY-5188: tetrapyrrole biosynthesis I (from glutamate), HEME-BIOSYNTHESIS-II: heme b biosynthesis I (aerobic), PWY-5367: petroselinate biosynthesis, DTDPRHAMSYN-PWY: dTDP-&beta;-L-rhamnose biosynthesis, and PWY-6471: peptidoglycan biosynthesis IV (Enterococcus faccium).

In various embodiments, the microbial signature indicative of ASD is an increase in relative abundances of biological pathways including one or more of PWY0-1298: superpathway of pyrimidine deoxyribonucleosides degradation, PWY-7013: (S)-propane-1,2-diol degradation, PWY-7456: &beta;-(1,4)-mannan degradation, METHGLYUT-PWY: superpathway of methylglyoxal degradation, GLUCARDEG-PWY: D-glucarate degradation I, PWY-6612: superpathway of tetrahydrofolate biosynthesis, PWY-5840: superpathway of menaquinol-7 biosynthesis, P161-PWY: acetylene degradation (anaerobic), FOLSYN-PWY: superpathway of tetrahydrofolate biosynthesis and salvage, ASPASN-PWY: superpathway of L-aspartate and L-asparagine biosynthesis, PWY-5971: palmitate biosynthesis (type II fatty acid synthase), PWY0-1297: superpathway of purine deoxyribonucleosides degradation, PWY-6588: pyruvate fermentation to acetone, GALACTARDEG-PWY: D-galactarate degradation I, PWY-6305: superpathway of putrescine biosynthesis, PWY4LZ-257: superpathway of fermentation (Chlamydomonas reinhardtii), GLUCARGALACTSUPER-PWY: superpathway of D-glucarate and D-galactarate degradation, PWY66-409: superpathway of purine nucleotide salvage, PWY-5154: L-arginine biosynthesis III (via N-acetyl-L-citrulline), PWY-5659: GDP-mannose biosynthesis, THISYNARA-PWY: superpathway of thiamine diphosphate biosynthesis III (cukaryotes), PWY-7003: glycerol degradation to butanol, CENTFERM-PWY: pyruvate fermentation to butanoate, PWY-5189: tetrapyrrole biosynthesis II (from glycine), PWY-2942: L-lysine biosynthesis III, HSERMETANA-PWY: L-methionine biosynthesis III, HEMESYN2-PWY: heme b biosynthesis II (oxygen-independent), RHAMCAT-PWY: L-rhamnose degradation I, GLUCUROCAT-PWY: superpathway of &beta;-D-glucuronosides degradation, PWY-6527: stachyose degradation, PWY0-162: superpathway of pyrimidine ribonucleotides de novo biosynthesis, PWY-5497: purine nucleobases degradation II (anaerobic), PWY-6284: superpathway of unsaturated fatty acids biosynthesis (E. coli), PWY-6317: D-galactose degradation I (Leloir pathway), P185-PWY: formaldehyde assimilation III (dihydroxyacetone cycle), PWY-6470: peptidoglycan biosynthesis V (&beta;-lactam resistance), PWY-7242: D-fructuronate degradation, PWY-5304: superpathway of sulfur oxidation (Acidianus ambivalens), ARG+POLYAMINE-SYN: superpathway of arginine and polyamine biosynthesis, PWY-5897: superpathway of menaquinol-11 biosynthesis, PWY-7357: thiamine phosphate formation from pyrithiamine and oxythiamine (yeast), GLYCOGENSYNTH-PWY: glycogen biosynthesis I (from ADP-D-Glucose), PWY-5918: superpathway of heme b biosynthesis from glutamate, PWY-5188: tetrapyrrole biosynthesis I (from glutamate), HEME-BIOSYNTHESIS-II: heme b biosynthesis I (aerobic), PWY-5367: petroselinate biosynthesis, DTDPRHAMSYN-PWY: dTDP-&beta;-L-rhamnose biosynthesis, and PWY-6471: peptidoglycan biosynthesis IV (Enterococcus faecium).

In various embodiments, the microbial signature indicative of ASD is a decrease in relative abundances of biological pathways including one or more of PWY0-1298: superpathway of pyrimidine deoxyribonucleosides degradation, PWY-7013: (S)-propane-1,2-diol degradation, PWY-7456: &beta;-(1,4)-mannan degradation, METHGLYUT-PWY: superpathway of methylglyoxal degradation, GLUCARDEG-PWY: D-glucarate degradation I, PWY-6612: superpathway of tetrahydrofolate biosynthesis, PWY-5840: superpathway of menaquinol-7 biosynthesis, P161-PWY: acetylene degradation (anaerobic), FOLSYN-PWY: superpathway of tetrahydrofolate biosynthesis and salvage, ASPASN-PWY: superpathway of L-aspartate and L-asparagine biosynthesis, PWY-5971: palmitate biosynthesis (type II fatty acid synthase), PWY0-1297: superpathway of purine deoxyribonucleosides degradation, PWY-6588: pyruvate fermentation to acetone, GALACTARDEG-PWY: D-galactarate degradation I, PWY-6305: superpathway of putrescine biosynthesis, PWY4LZ-257: superpathway of fermentation (Chlamydomonas reinhardtii), GLUCARGALACTSUPER-PWY: superpathway of D-glucarate and D-galactarate degradation, PWY66-409: superpathway of purine nucleotide salvage, PWY-5154: L-arginine biosynthesis III (via N-acetyl-L-citrulline), PWY-5659: GDP-mannose biosynthesis, THISYNARA-PWY: superpathway of thiamine diphosphate biosynthesis III (eukaryotes), PWY-7003: glycerol degradation to butanol, CENTFERM-PWY: pyruvate fermentation to butanoate, PWY-5189: tetrapyrrole biosynthesis II (from glycine), PWY-2942: L-lysine biosynthesis III, HSERMETANA-PWY: L-methionine biosynthesis III, HEMESYN2-PWY: heme b biosynthesis II (oxygen-independent), RHAMCAT-PWY: L-rhamnose degradation I, GLUCUROCAT-PWY: superpathway of &beta;-D-glucuronosides degradation, PWY-6527: stachyose degradation, PWY0-162: superpathway of pyrimidine ribonucleotides de novo biosynthesis, PWY-5497: purine nucleobases degradation II (anaerobic), PWY-6284: superpathway of unsaturated fatty acids biosynthesis (E. coli), PWY-6317: D-galactose degradation I (Leloir pathway), P185-PWY: formaldehyde assimilation III (dihydroxyacetone cycle), PWY-6470: peptidoglycan biosynthesis V (&beta;-lactam resistance), PWY-7242: D-fructuronate degradation, PWY-5304: superpathway of sulfur oxidation (Acidianus ambivalens), ARG+POLYAMINE-SYN: superpathway of arginine and polyamine biosynthesis, PWY-5897: superpathway of menaquinol-11 biosynthesis, PWY-7357: thiamine phosphate formation from pyrithiamine and oxythiamine (yeast), GLYCOGENSYNTH-PWY: glycogen biosynthesis I (from ADP-D-Glucose), PWY-5918: superpathway of heme b biosynthesis from glutamate, PWY-5188: tetrapyrrole biosynthesis I (from glutamate), HEME-BIOSYNTHESIS-II: heme b biosynthesis I (aerobic), PWY-5367: petroselinate biosynthesis, DTDPRHAMSYN-PWY: dTDP-&beta;-L-rhamnose biosynthesis, and PWY-6471: peptidoglycan biosynthesis IV (Enterococcus faecium).

In various embodiments, the microbial signature indicative of ASD is an increase in relative abundances of biological pathways including one or more of PWY0-1298: superpathway of pyrimidine deoxyribonucleosides degradation, PWY-7013: (S)-propane-1,2-diol degradation and/or PWY-7456: &beta;-(1,4)-mannan degradation.

In various embodiments, the microbial signature indicative of ASD is a decrease in relative abundances of biological pathways including one or more of PWY0-1298: superpathway of pyrimidine deoxyribonucleosides degradation, PWY-7013: (S)-propane-1,2-diol degradation and/or PWY-7456: &beta;-(1,4)-mannan degradation.

In one embodiment, the microbial signature indicative of ASD is an increase in relative abundance of PWY0-1298: superpathway of pyrimidine deoxyribonucleosides degradation.

In one embodiment, the microbial signature indicative of ASD is a decrease in relative abundance of PWY0-1298: superpathway of pyrimidine deoxyribonucleosides degradation.

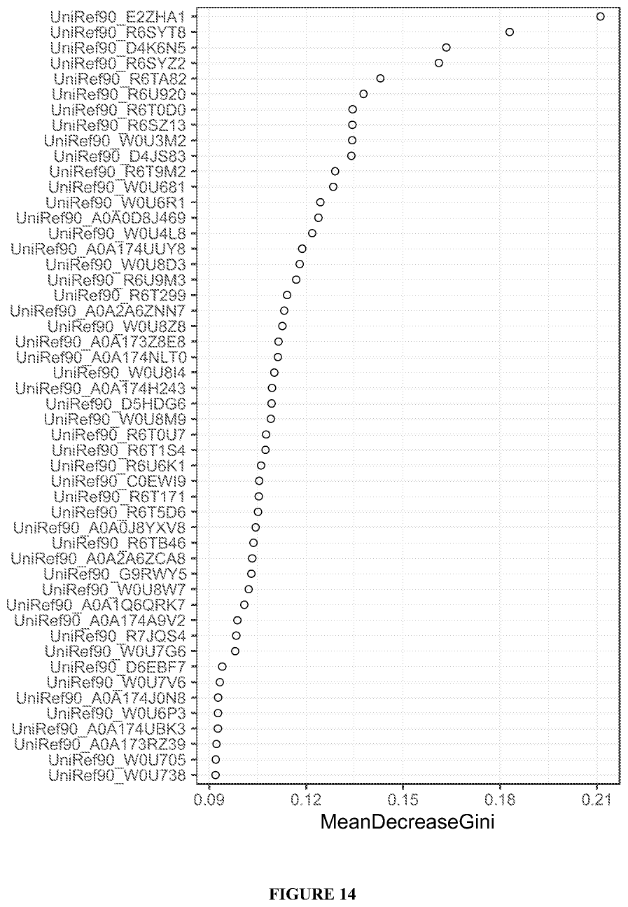

In various embodiments, the microbial signature indicative of ASD is presence, absence or relative abundances of gene families including one or more of those set forth in FIG. 14.

In various embodiments, the microbial signature indicative of ASD is presence, absence or relative abundances of gene families including one or more of UniRef90_E2ZHA1, UniRef90_R6SYT8, UniRef90_D4K6N5, UniRef90_R6SYZ2, UniRef90_R6TA82, UniRef90_R6U920, UniRef90_R6TOD0, UniRef90_R6SZ13, UniRef90_W0U3M2, UniRef90_D4JS83, UniRef90_R6T9M2, UniRef90_W0U681, UniRef90_W0U6R1, UniRef90_A0A0D8J469, UniRef90_W0U4L8, UniRef90_A0A174UUY8, UniRef90_W0U8D3, UniRef90_R6U9M3, UniRef90_R6T299, UniRef90_A0A2A6ZNN7, UniRef90_W0U8Z8, UniRef90_A0A173Z8E8, UniRef90_A0A174NLT0, UniRef90_W0U814, UniRef90_A0A174H243, UniRef90_D5HDG6, UniRef90_W0U8M9, UniRef90_R6T0U7, UniRef90_R6T1S4, UniRef90_R6U6K1, UniRef90_C0EW19, UniRef90_R6T171, UniRef90_R6T5D6, UniRef90_A0A0J8YXV8, UniRef90_R6TB46, UniRef90_A0A2A6ZCA8, UniRef90_G9RWY5, UniRef90_W0U8W7, UniRef90_A0A1Q6QRK7, UniRef90_A0A174A9V2, UniRef90_R7JQS4, UniRef90_W0U7G6, UniRef90_D6EBF7, UniRef90_W0U7V6, UniRef90_A0A174JON8, UniRef90_W0U6P3, UniRef90_A0A174UBK3, UniRef90_A0A173RZ39, UniRef90_W0U705 and UniRef90_W0U738.

In various embodiments, the microbial signature indicative of ASD is an increase in relative abundances of gene families including one or more of UniRef90_E2ZHA1, UniRef90_R6SYT8, UniRef90_D4K6N5 and/or UniRef90_R6SYZ2.

In various embodiments, the microbial signature indicative of ASD is a decrease in relative abundances of gene families including one or more of UniRef90_E2ZHA1, UniRef90_R6SYT8, UniRef90_D4K6N5 and/or UniRef90_R6SYZ2.

In one embodiment, the microbial signature indicative of ASD is an increase in relative abundance of the gene family UniRef90_E2ZHA1.

In one embodiment, the microbial signature indicative of ASD is a decrease in relative abundance of the gene family UniRef90_E2ZHA1.

In various embodiments, the methodology of the present invention includes correlating metadata from a standardized survey with an identified microbial signature associated with a particular disease. Such surveys may include any know in the art, such as those associated with ASD, including, for example, Social Responsiveness Scale (SRS-2), Parent Global Impressions (PGIA), Daily Bristol Stool Records, Food Questionnaire, Diet Evaluation, Childhood Autism Rating Scale, Gastrointestinal Symptom Rating Scale (GSRS). Such surveys are combined with metagenomic and/or metabolomic data to access risk or severity of ASD.

In various embodiments, the methodology of the invention includes determining a microbiome score. Scoring of the microbiome signature overall uses a similar decision tree, algorithm, artificial intelligence, script, or logic tree as represented in Table 1. This system enables a score that helps a user understand how healthy their gut microbiome is and if they need to take action on a few or many challenges found. Challenges can include but not limited to, identification of known pathogenic organisms, count and identification of opportunistic pathogens, latent organisms known to cause pathogenic affects when given opportunity, lack of support for good microbial environment but their composition or lack of key strains, overall diversity and count of unique organisms found in top 10 and or organisms with greater than 0.1% prevalence.

Diversity cut offs were determined from an aggregate of sample analysis and a cutoff is determined at x relative abundance. For example, if x=0.1% then 352 unique organisms make up the average healthy profile. Then apply standard deviations around this number and using a Gaussian distribution and percentile under the curve analysis we can score how close to the average diversity number from our database average. The lower your diversity number and further away from the average you are then the less that microbiome would score. The higher the number and the greater your diversity is the more that microbiome would score. This type of scoring categories along with probiotic score will determine a number and visual metered score for the custom to understand how healthy their microbiome is. An example of the graphic visualization is included below. Where low is equal to low microbiome quality and high is equal to high microbiome quality and score. Low->30 out of 100, Med>65 out of 100, High=65 or greater out of 100.

An example of a scoring and probiotic formula algorithm is included in Table 1 below. Table 1 can be represented as decision tree, algorithm, artificial intelligence, script, or logic tree. The function of such decision tree, algorithm, artificial intelligence, script, or logic tree would be output a score of wellness of the individual microbiome as related to probiotics detected and to provide formulation and dosing recommendations for probiotic usage.

TABLE 1
Example Decision Table for Scoring Including the Utilization of a Probiotic Strain
Database, Metagenomic Analysis Database, and Literature Curation Database.

| Criteria | | Criteria | |
|---|---|---|---|
| Number | Criteria | Answer | Score or Inclusion/Exclusion |
| 1 | Greater than 100 | Yes | If yes then include |
| | reads | | |
| 2 | Greater than 50% of | Yes | |
| | total probiotic reads | | |
| 3 | Greater than 10,000 | Yes | If yes do not include in probiotic formula |
| | reads | | |
| 4 | Greater than 50% of | No | |
| | total reads | | |
| 5 | Greater than 30,000 | Yes | If yes do not include in probiotic formula |
| | reads | | |
| 6 | Greater than 30,000 | Yes | If x > 5 then score +20, x > 3 score 10, x > 1 |
| | reads for x number | | score 5 |
| | of probiotics | | |
| 7 | Total number of | x | If x > 10 then score +20, x > 10 then score 10, |
| | microbes above 100 | | x > 5 score 5 |
| | reads (count) | | |
| 8 | Query for probiotic | Yes | Include in formula at 20 CFU/g or greater |
| | strains and output | | |
| | where 1 = yes and 4 | | |
| | is no and 6 is no and | | |
| | the number of reads | | |
| | is less than 1000 | | |
| 9 | If bacillus | Yes | Do not include |
| 10 | If lactobacillus | Yes | If x > 10000 score +20, if x > 1000 score +10, |
| | acidophilus greater | | if x > 100 score +5 |
| | than x reads | | |
| 11 | If bacillus genus | Yes | If x > 1000 score +20, if x > 100 score +10, if x > |
| | greater than x reads | | 10 score +5 |
| 12 | If Saccharomyes | Yes | If x > 1000 score +20, if x > 100 score +10, if x > |
| | boulardi greater than | | 10 score +5 |
| | x reads | | |
| 13 | If infant if nursing | Yes | If x > 10 then score +5, x > 30% then score +10, |
| | and bifidobacterium | | x > 50% then score +20, x > 70% then |
| | infantis > x % | | score +30 |
| 14 | If not infant, not | Yes | If x > 20 then score +5, if x > 10 then score +10, |
| | child and | | if x < 10 then score +20 |
| | bifidobacterium | | |
| | infantis > x % | | |

| | |
|---|---|
| 15 | Query to probiotic function, if function table is equal to health phenotype or |
| | healthDx then include in formula unless 3 or 5 = yes |

In various embodiments, the methodology of the invention includes determining one or more microbiome scores to assess health. In various embodiments, the microbiome score is one or more of an immunity score, a diversity index score, a joint health index score, a longevity score, a microbiome diversity score, a Firmicutes/Bacteroidetes Ratio score, dysbiosis/inflammation score, a disease protection score, and a bad microbes score.

In embodiments, the diversity index score is determined by calculating the richness and evenness characteristics of a community, often calculated as a specific “diversity index”.

In embodiments, the joint health index score is calculated based on certain criteria including the presence/absence of microbes belonging to the Genus Prevotella.

In embodiments, the Firmicutes/Bacteroidetes Ratio score determines risk for a number of conditions, including but not limited to diabetes (Type 2), cardiovascular disease, and metabolic disease. People with a normal weight tend to have a higher ratio of Firmicutes to Bacteroidetes as Firmicutes tend to produce butyrate, a special compound that can increase insulin sensitivity, regulate metabolism, and has anti-inflammatory properties. This score is defined by calculating the ratio and its association to disease. A lower score indicates closer association to disease conditions.

In embodiments, the dysbiosis/inflammation score indicates the presence/absence of the key microbes that are indicators of the gut health. In one embodiment, Faccalibacterium is known to have anti-inflammatory properties and its presence indicates good gut health.

In embodiments, the microbiome diversity score determines the overall amount of individual bacteria from each of the bacterial species present in a gut microbiome. In embodiments, the microbiome diversity score includes an alpha diversity comparison used to compare gut diversity in a subject with that of a healthy subject. It is a measure of microbiome diversity applicable to a single sample, and many indices exist, like presence/absence of certain keystone species and microbial abundance level, each reflecting different aspects of community heterogeneity. In embodiments, the microbiome diversity score includes a microbiome abundance by genus index calculating the percentage of individual genus presence by percentage.

In another aspect, the invention provides a method of treating a disease or disorder, such as IBS or ASD. The method includes diagnosing the subject as having, or at risk or having, a disease or disorder using the method of the invention, and administering the subject a therapeutic and/or dietary supplement to treat the disease or disorder in the subject. In some embodiments, the method includes administering a custom dietary supplement to the subject. In various embodiments, the custom dietary supplement may include a probiotic, pre-biotic, metabolite, enzyme, vitamin, mineral, natural extract and/or botanical. For example, the method may include administering the subject a customized probiotic formulation of the invention.

In some embodiments, the present invention may be used to screen the gut microbiome of a given subject and then custom tailor a food or diet regime that would enable them to improve the quality of their health for aspects of nutritional balance, improved microbial gut profile, and absorption of nutrients.

In some embodiments, the present invention may be used to monitor probiotic treatment in subjects. For example, prior to treatment with a probiotic, a sample obtained from the digestive tract of a subject may be obtained and the genetic material of the microbes therein extracted as disclosed herein and subjected to metagenomics analysis. Then during and/or after treatment with a given probiotic, a second sample may be obtained from the digestive tract of the subject and the genetic material of the microbes in the second sample extracted as disclosed herein and subjected to metagenomics analysis, the results of which are compared to the results of the metagenomics analysis of the first sample. Then, based on the comparative results, the probiotic treatment of the subject may be modified to obtain a desired population of microbes in the gut of the subject. For example, a probiotic that comprises a microbe whose amount is desired to be increased in the gut of the subject may be administered to the subject.

In some embodiments, the fecal sample may be mixed or cultured for determination of metabolomic of microbial fecal community. Metabolomic profile can then be used to determine probiotic strains that would benefit the individual. Examples of metabolomic profiles include those affecting energy metabolism, nutrient utilization, insulin resistance, adiposity, dyslipidemia, inflammation, short-chain fatty acids, organic acids, cytokines, neurotransmitters chemicals or phenotype and may include other metabolomic markers.

In one embodiment, based on the microbiome content in the gut of the subject and any desired changes thereto, one may select one or more probiotics that contain the microbes that are desired to be increased and/or maintained in the subject's microbiome health. In one embodiment, based on the microbiome content in the gut of the subject and any desired changes thereto, one may select one or more probiotics that contain the microbes that are desired to be increased and/or maintained in the subject's gut balance in relation to the macronutrient content they are getting from their food source as recorded by survey information from the individual directly or by the present invention of gut organism nucleic acid analysis.

Where the microbiome represents a full picture of their microbiota and the organisms contained in them from bacteria, fungi, viruses, phages, and parasites. For example, using the methods described herein, a subject's gut microbiome is determined to contain 25% A and 75% B, Probiotic 1 is determined to contain 75% A and 25% B and Probiotic 2 is determined to contain 25% A and 75% B. If the subject's gut microbiome is desired to be maintained, one would select Probiotic 2 for administering to the subject. However, if the amounts of A and B in the subject's gut are desired to be 50/50, one may select both Probiotics 1 and 2 to be administered to the subject. Alternatively, one may select Probiotic 1 to be administered to the subject until the amounts of A and B in the subject's gut reaches 50/50. In some embodiments, one may custom tailor a probiotic formulation, e.g., containing equal, varying, or diverse amounts of A and B or other probiotic strains, for administration to the subject. Calculation models utilizing relative abundance of the microbes present in an individual's gut will help determine the type, dose, and cocktail of microbes to include in the probotic. For example, if it is determined that organism A is reduced or absent compared to the general population or previous microbiome analysis, then we would provide probiotic or prebiotics that would increase the concentration of organism A. This prebiotic or probiotic may be the exact organism A or another organism what would support the growth of organism A. The dose given would consider relative abundance of organisms in the individual, performance characteristics of the prebiotic/probiotic such as growth rate, compatibility, receptors or receptor density, genes, or expression patterns, or metabolomic products.

Custom tailored probiotics may not be in equal amounts but are formulated based on relative abundance detected from the individual gut/fecal sample. These formulations are geared to modulate the microbiome to a healthy status. The healthy status of a microbiome is determined by the use of existing aggregate private and public databases such as metaHIT™, Human Microbiome Project™, American Gut Project™, and the like. The healthy status may also be determined individually when a person has no known issues and is in good health, from a blood biomarker checkup perspective, and then has their full microbiome profile completed. After one or several microbiome signatures have been completed then the average of some/all of the microbes found can be understood for that individual and variances from that average can be accessed to determine if they are in dysbiosis. Microbiome profiles can be aggregated into groups that are then assigned a barcode for rapid bioinformatic assignment. Groups can be created by single or multiple phenotypic, diagnostic, or demographic information related to the individual from which the sample was collected from. A unique group can be determined from another group by using statistical models such as linear distance calculations, diversity values, classifiers such as C4.5 decision tree, or principal component analysis an comparing to an aggregate known population such as “normals” defined by the Human Microbiome Project or American Gut Project.

Thus, in some embodiments, the present invention may be used to screen the gut microbiome of a given subject and then custom tailor a probiotic regimen to the given subject based on the subject's gut microbiome.

In some embodiments, the present invention may be used to restore a subject's gut flora and/or fauna to homeostasis after an event that has caused a shift in the subject's microbiota from balanced microbiome to one that is causing or may be causing negative side effects, disorders, and/or disease. Thus, in some embodiments, a ratio of a first given microbe to a second given microbe in the gut of a subject is determined using the methods described herein and then if the ratio is undesired or abnormal, the subject is administered a treatment to modify the ratio to be a desired ratio. In some embodiments, the amount of a first given microbe in a gut of a subject relative to the total amount of all the microbes in the gut of the subject is determined using the methods described herein and then if the relative amount of the first given microbe is undesired or abnormal, the subject is administered a treatment to modify the amount to be a desired amount. Re-testing of their gut microbiome maybe used to determine well they are adhering to the macronutrient and food guidance. Such treatments include administering to the subject: a probiotic containing one or more microbes whose amounts are desired to be increased in the gut of the subject, an antimicrobial agent, e.g., an antibiotic, an antifungal, an antiviral, etc., to kill or slow the growth of a microbe or microbes whose amounts are desired to be decreased in the gut of the subject, a diet and/or a dietary supplement that supports the growth or maintenance of a healthy gut microbiome, e.g., a prebiotic, magnesium, fish oil, L-glutamine, vitamin D, etc., and the like.

Methods for data analysis according to various aspects of the present invention may be implemented in any suitable manner, for example using a computer program operating on the computer system. An exemplary analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The analysis system according to various aspects of the present invention and its various elements provide functions and operations to facilitate microbiome analysis, such as data gathering, processing, analysis, reporting and/or diagnosis. The present analysis system maintains information relating to microbiomes and samples and facilitates analysis and/or diagnosis. For example, in the present embodiment, the computer system executes the computer program, which may receive, store, search, analyze, and report information relating to the microbiome. The computer program may comprise multiple modules performing various functions or operations, such as a processing module for processing raw data and generating supplemental data and an analysis module for analyzing raw data and supplemental data to generate a models and/or predictions.

The analysis system may also provide various additional modules and/or individual functions. For example, the analysis system may also include a reporting function, for example to provide information relating to the processing and analysis functions. The analysis system may also provide various administrative and management functions, such as controlling access and performing other administrative functions.

The use of the singular can include the plural unless specifically stated otherwise. As used in the specification and the appended claims, the singular forms “a”, “an”, and “the” can include plural referents unless the context clearly dictates otherwise. The use of “or” can mean “and/or” unless stated otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in the medical diagnosis context, the present invention may be practiced in conjunction with any number of applications, environments and data analyses; the systems described herein are merely exemplary applications for the invention.

To the extent necessary to understand or complete the disclosure of the present invention, all publications, patents, and patent applications mentioned herein are expressly incorporated by reference therein to the same extent as though each were individually so incorporated.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

Alterations in Gut Microbiome Composition and Function in Irritable Bowel Syndrome and Increased Probiotic Abundance with Daily Supplementation

Irritable bowel syndrome (IBS) is characterized by chronic gastrointestinal discomfort and abdominal pain with changes in bowel habits or stool consistency. IBS affects approximately 11.5% of the population, depending on the country or region. Because of the high prevalence of IBS, symptoms contribute to changes in quality of life and increases in healthcare and economic burden. There are four subtypes based on the symptoms people experience, IBS-C (constipation), IBS-D (diarrhea), IBS-M (mixed), or unspecified. Individuals with IBS-M experience alternating symptoms of chronic diarrhea and constipation. The criterion for diagnosis is symptom based and codified in the Rome IV criteria; there is not yet consensus on the underlying etiology of IBS. In addition, there are different factors that contribute to the varying symptoms of IBS, including diet, immune response, host genetics, environmental stress, gut microbiome composition, and dysbiosis.

Currently, the role of the gut microbiome in individuals with IBS remains poorly understood. A “healthy” gut microbiome may be undefined, but there are microorganisms associated with an unhealthy microbiome, including microorganisms that induce inflammation or dysbiosis that contribute to the symptoms associated with IBS. Changes in microbiome composition also impacts the functional potential and metabolism of the microbiome which may in turn affect host physiology. Studies indicate individuals experiencing IBS-C show microbiome signatures such as increased Pseudomonas and Bacteroides thetaiotamicron with a depletion of Paraprevotella, significant associations with Fusobacterium nucleatum and Meganomoas hypermegale, and pathways of sugar and amino acid metabolism. While microbiomes in IBS-C were characterized with the biosynthetic pathways for sugar and amino acid metabolism, subjects with IBS-D had microbes that predominated the pathways for nucleotides and fatty acid synthesis. Amplicon studies have also described an enrichment of Clostridiales, Prevotella, and Enterobacteriaceae, reduced microbial richness, and the presence of methanogens in IBS. However, amplicon studies can be subject to amplification bias, yielding variable results and do not resolve species level taxonomic classification. Alternatively, several studies limited by sample size and methodology have not shown a difference between a healthy cohort and individuals with IBS.

Because of the differences in IBS symptoms people experience and individual nature of the syndrome, there is no standardized treatment or dietary recommendations to alleviate IBS symptoms. The antibiotic rifaximin has been shown to be an effective treatment for IBS-D. However, rifaximin is not effective for all IBS subtypes and antibiotic usage may be associated with an increased risk for IBS. There are additional options for treatment, including pharmaceutical options and fecal transplants, but these options are not always feasible and can be invasive. Administration of live microbial organisms, in the form of probiotics has gained popularity with patients to alleviate their symptoms. Probiotics can alter the microbiome of patients with and without IBS, depending on their endogenous microbiomes. Microbes not present in the current gut microbiome can also be re-established through probiotic supplementation. In individuals with IBS, there is correlative depletion of Bifidobacterium and Lactobacillus. Therefore, re-introduction of probiotics into the gut microbiomes of individuals with IBS, may lead to phenotypic changes. Clinical trials have demonstrated the reduction of symptoms associated with IBS with probiotic supplementation. When subjects with IBS-D were treated with Bifidobacterium longum, B. bifidum, B. lactis, B. infantis, and Lactobacillus acidophilus, there was a change in inflammation related metabolites. Individuals with IBS on a gluten-free diet with probiotic supplementation of Lactobacillus and Bifidobacterium spp. saw an overall improvement in symptoms. Probiotic supplementation has also reduced stomach pain and improved stool consistency in individuals with IBS.

This Example presents a large-scale metagenomic study to characterize and compare the microbiome composition and functional potential of healthy controls and individuals with IBS. In addition, we collected longitudinal timepoints from individuals with IBS on daily prebiotic and probiotic supplementation. The primary goals were to 1) identify metagenomic signatures associated with IBS and 2) investigate the microbiome effects of precision prebiotics and probiotics on individuals with IBS. Included in each made-to-order formulation are 4-8 probiotic strains from a biobank and 1-3 prebiotics, each at different concentrations from a biobank of over 100 possible ingredients supported by the clinical literature. Whole genome shotgun sequencing allows for species level resolution and identification of functional potential. This method reduces amplification bias in amplicon studies and does not require the prediction of potential pathways to investigate functional potential. The inventors also investigated whether the use of traditional tools in gross microbiome analysis can be used to determine changes to the microbiome after probiotic supplementation. It was hypothesized that metagenomic features distinguish healthy and IBS microbiome subtypes and that daily probiotic supplementation modulates the microbiomes of the individuals with IBS.

This study included metagenomic sequencing of stool samples from subjects with the predominant subtypes of IBS and a healthy cohort. Longitudinal samples were collected from individuals with IBS who took daily made-to-order precision probiotic and prebiotic supplementation throughout the duration of the study.

An estimated 35 million people in the United States and 11% of the population globally are affected by IBS. Immunity, genetics, environment, diet, small intestinal bacterial overgrowth (SIBO), and the gut microbiome are all factors that contribute to the onset or triggers of IBS. With strong supporting evidence that the gut microbiome may influence symptoms associated with IBS, elucidating the important microbes that contribute to the symptoms and severity is important to make decisions for targeted treatment. As probiotics have become more common in treating the symptoms of IBS, identifying effective probiotics may help inform future studies and treatment.

### Materials and Methods

### Participants and Sample Collection.

Users of a (Sun Genomics, San Diego) commercial at-home gut microbiome testing component (Floré™ Gut Health Test Kit) submitted a stool sample for metagenomic sequencing. The stool sample was collected by the user with provided gut testing kit instructions. Samples were collected in accordance with IRB #SG-04142018-001 with informed consent form 001-B. For the first collection device, a sterile swab was used to collect and store the stool sample in a collection tube with stabilization buffer. The second sample was collected via the Easi-Collect™ device (GE). Samples were mailed via FedEx™ to the Sun Genomics lab for analysis.

A total of 611 participants were included in this study (Table 2). All participants completed a health and diet survey that asked questions about health status and dietary preferences. The control population included in this study was self-reported as healthy with no listed comorbidities with a BMI range from 18.5-25 (Table 2). The IBS population was also self-reported and included the symptoms associated with the syndrome, including constipation, diarrhea, a mix of both constipation and diarrhea, or unspecified.

Table 2. Subject demographics. “Healthy” controls are self-reported as healthy subjects with no existing comorbidities. Subjects with IBS are also self-reported. The subtype designation is based on the subject symptoms. For the alternating designation, subjects experienced symptoms of constipation and diarrhea. Cohort populations are further classified by gender. Average and standard deviation of age groups are listed next to each population.

| | | Female | | Male | | Unspecified | |
|---|---|---|---|---|---|---|---|
| Phenotype | Subjects | (Age +/− SD) | | (Age +/− SD) | | (Age +/− SD) | |
| Healthy | 122 | 54 | (44 +/− 13) | 52 | (44 +/− 12.6) | 16 | (41.9 +/− 9.2) |
| IBS (Total) | 490 | 302 | (46.5 +/− 15.5) | 158 | (41.6 +/− 15.3) | 31 | (43.3 +/− 16.9) |
| IBS-C | 185 | 126 | (45.5 +/− 14.9) | 50 | (37.4 +/− 13.5) | 9 | (41.7 +/− 13.1) |
| (Constipation) | | | | | | | |
| IBS-D (Diarrhea) | 86 | 50 | (41.9 +/− 14.5) | 32 | (44.3 +/− 16.5) | 4 | (40.7 +/− 12.1) |
| IBS-M | 88 | 58 | (45.4 +/− 15.6) | 26 | (37.6 +/− 12.4) | 4 | (41.5 +/− 14) |
| (Alternating) | | | | | | | |
| IBS-U | 131 | 64 | (53.6 +/− 15.6) | 49 | (46.5 +/− 16.3) | 18 | (45.6 +/− 21.5) |
| (Unspecified) | | | | | | | |

### Metagenomic Sequencing and Analysis.

For DNA extractions, samples were first processed with a tissue homogenizer and then lysed with a lysis buffer and proteinase K. DNA was extracted and purified with a proprietary method (U.S. Pat. Nos. 10,428,370 and 10,837,046). Library preparation was performed with DNA sonication, end-repair, and adaptor ligation with NEBNext™ reagents. Size selection was performed with MagJet™ Magnetic Beads according to manufacturer instructions. Library quantitation was performed with qPCR and sequenced on an Illumina NextSeq™ 550 (Illumina, San Diego). After sequencing, reads were quality filtered and processed. Metagenomic reads were decontaminated from human reads using Bowtie2™. Next, reads were aligned to a hand curated database of over 23,000 species. Humann3™ was used for pathway analysis. Pathway abundance was normalized to copies per million (cpm).

### Statistical Analyses.

All statistical analyses were performed in R. Principal coordinates analysis was performed with a Bray-Curtis dissimilarity matrix to compare between sample diversities. Within sample diversity was calculated with the Shannon diversity index. To calculate variance between samples based on metadata classifications, permutational multivariate analysis of variance (PERMANOVA) was performed with the “adonis” function from the “vegan” package. Specifically, the influence of health status was computed across the microbiome composition and pathway abundance profiles. MaasLin2™ was used for distinguishing pathway features between healthy and IBS subtypes.

### Results

There was significant variation explained in the microbiome between the healthy and IBS cohorts. Individuals with IBS had a lower gut microbiome diversity and a reduction of anti-inflammatory microbes compared to the healthy controls. Eubacterium rectale and Faecalibacterium prausnitzii were associated with healthy microbiomes while Shigella species were associated with IBS. In the longitudinal dataset, there was a significant difference in microbiome composition between timepoints 1 and 3. There was also a significant increase in the overall microbiome score and probiotic species relative abundances used to target the symptoms associated with IBS.

### IBS and Healthy Subject Demographics.

A total of 611 subjects were included in this study. Subjects without reported comorbidities and self-reported as healthy were included as the healthy control population. In total, there were 489 subjects with IBS and 122 subjects for the healthy control population (Table 2). In addition, longitudinal samples from people with IBS were assessed to identify whether there were specific microbiome changes during the course of prebiotic and probiotic supplementation. These healthy and IBS subjects were also assigned an internal health index score for their initial microbiome profile and subsequent timepoints. The probiotics were designed to be a part of a daily regiment for all subjects. Longitudinal timepoints were approximately 4 months apart. Of the 489 IBS subject population, 134 subjects had at least 2 timepoints, 56 subjects had 3 timepoints, 28 subjects with 4 timepoints, 15 subjects with 5 timepoints, 5 subjects with 6 timepoints, and 1 subject with 7 timepoints.

Reduced microbial diversity and microbial signatures associated with IBS.

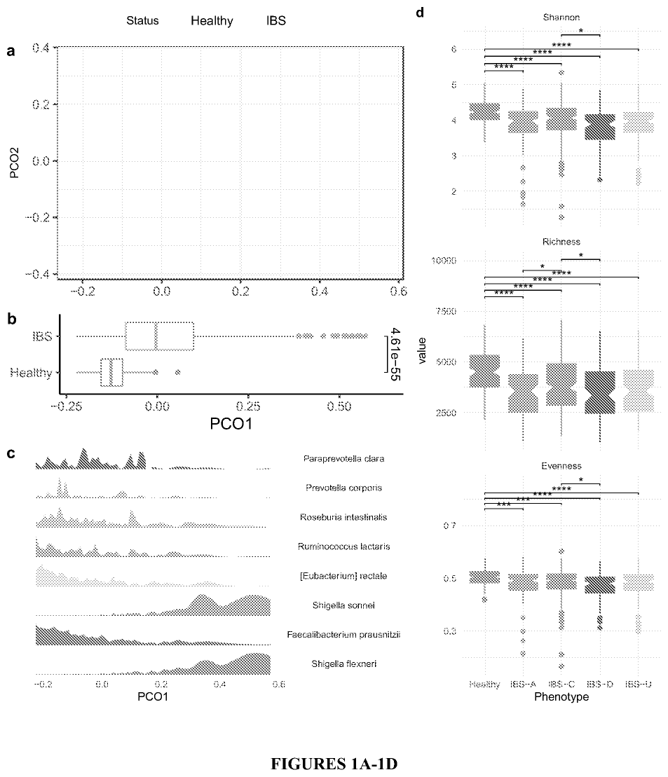

First, to compare the microbial community composition between the IBS and healthy control populations, a principal coordinates analysis was performed to visualize the beta diversity between the two cohorts (FIG. 1). All healthy control microbiome samples clustered tightly together, while there was a spread of IBS samples that clustered around and away from the healthy control microbiome samples. The differences in the IBS samples that clustered away from healthy were distinguished by their increased relative abundances of Enterobacterales species and reduction in Eubacterium rectale and Faecalibacterium prausnitzii (FIG. 1). The top 10 microbes that distinguish healthy and IBS were determined by random forest and were plotted along the second principal coordinate axis to show the spread of sample clustering between the healthy and IBS microbiomes. Next, when calculating alpha diversity metrics, there was a significant reduction in the Shannon index, richness, and evenness in IBS subtypes compared to the healthy control population (FIG. 1).

Based on whole genome shotgun metagenomic sequencing, there were microbial signatures that distinguish the healthy control and IBS populations. Using a permutated multivariate analysis of variance, there was significant variation that explained the difference between the microbiome of healthy and IBS subtypes (R2=0.028, p<0.001). A random forest algorithm was used to identify the distinguishing microbes between healthy and IBS phenotypes. To identify microbial relative abundances within healthy or IBS subtypes, unpaired t-tests were calculated and p-values were adjusted for multiple testing corrections. Eubacterium rectale and Faecalibacterium prausnitzii were significantly increased in the healthy control population relative to all IBS subtypes (FIG. 2), while inflammatory species of Shigella were elevated in IBS (FIG. 2). Paraprevotella clara, Prevotella corporis, Roseburia intestinalis and Ruminococcus lactaris were significantly decreased in different IBS subtypes relative to the healthy control population (FIG. 2).

Functional profile of the gut microbiome of subjects with IBS and healthy.

To determine the functional profiles of the gut microbiome associated with IBS, we mapped the metagenomic reads against the MetaCyc™ database with Humann3™ to identify pathway abundances. There were a total of 471 pathways detected in the metagenomes of healthy and individuals with IBS. Multivariate linear association testing identified pathways that were associated with each of the IBS dominant subtypes relative to the healthy control cohort (FIG. 2). Pathways involved in tetrapyrrole biosynthesis from glycine, enterobacterial common antigen biosynthesis, NADP/NADPH interconversion, and the super pathway of heme b biosynthesis from glutamate were positively associated with IBS-A (FIG. 2). Methanogenesis from acetate was associated with IBS-C and IBS-D (FIG. 2). Pathways involved in the Bifidobacterium shunt, super pathway of glycerol degradation to 1,3-propanediol, and starch biosynthesis were associated with IBS-C(FIG. 2). Meanwhile, pathways associated with amino acid and ribonucleotide biosynthesis, polysaccharide degradation, and fermentation were associated with healthy microbiome functional profiles (FIG. 2).

Probiotics may modulate microbiome of subjects with IBS.

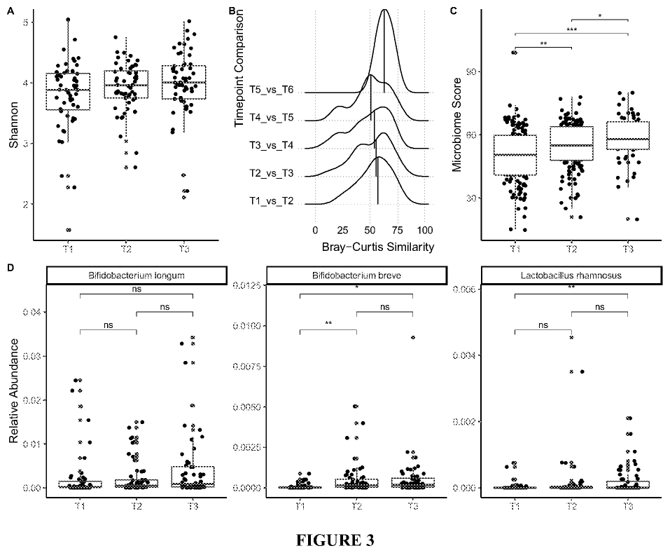

Within a subset of the IBS population, there were 134 individuals with at least two timepoints and 56 individuals with thee timepoints. The average number of days between timepoint 1 and 2 was 154.8±80.5 (standard deviation, SD) days, and timepoint 2 and 3 was 194.9±144.5 (SD) days. To investigate whether there were changes in alpha diversity across time, linear mixed effects models were computed to control for the effect from the individual. Based on the calculations on the longitudinal dataset controlling for the individual, there were no significant increases in the Shannon index, richness, or evenness. Although not significant, there may be an increase in the Shannon index across timepoints 1-3 (FIG. 3). Next, Bray-Curtis similarity of microbiome composition was calculated to investigate microbiome changes across time. There was no significant difference from one timepoint to the next (FIG. 3), or when comparing the first timepoint with each subsequent timepoint (data not shown). However, there was a shift in the median towards lower Bray-Curtis similarity indices across longitudinal timepoints 1-5 towards a lower similarity index (FIG. 3). To calculate microbiome variance across longitudinal samples, a permutated multivariate analysis of variance was performed across all timepoints. There was a significant difference between all longitudinal samples from timepoint 1 and timepoint 3 (R2=0.0088, p=0.035). Average days separating timepoints 1 and 3 were 335.9±170.5 (SD) days. When computing the variance for only subjects with both timepoints, there was not a significant amount of variation explained in the microbiome data. Taking into consideration the microbiome composition and health and diet survey information, we calculated the microbiome score for each sample and saw that there was a significant increase in the overall microbiome score across timepoints 1-3 (FIG. 3).

Because there were different subtypes associated with IBS included in our population, we investigated whether the individually formulated probiotics targeted towards relieving the symptoms of constipation and diarrhea were increased in abundance in the microbiomes of the IBS population. Each formulation contained approximately 4-8 probiotic strains, each at different concentrations. One of the common probiotics formulated for constipation was Bifidobacterium longum and the formulations for diarrhea included Bifidobacterium breve and Lactobacillus rhamnosus. In the longitudinal dataset, B. breve and L. rhamnosus significantly increased in abundance across time (FIG. 3). B. breve significantly increased from timepoint 1 to 2 and 3, but there was no significant change between the 2nd and 3rd timepoints (FIG. 3). L. rhamnosus was significantly increased in abundance at timepoint 3 compared to timepoint 1 (FIG. 3). There was not a significant increase in relative abundance of B. longum across timepoints 1-3.

### Discussion

Although IBS is prevalent across the population, the underlying factors contributing to the syndrome makes diagnosis and treatment difficult to define and standardize. Previous amplicon-based studies have identified changes in microbiome composition and diversity in individuals with IBS compared to a healthy control population. Concomitant with previous findings, our study corroborates the significant microbial community composition differences and diversity between healthy individuals and people with IBS. Unlike other studies, whole metagenome shotgun sequencing enabled us to identify pathways associated with the dominant subtypes of IBS. In addition, our precision probiotics for individuals with IBS showed a significant microbiome score improvement across time. Clinical studies that administer probiotics to individuals with IBS have shown reduced symptom severity and gut discomfort. Although a significant change in alpha or beta diversity in the longitudinal IBS profiles was not found with probiotic supplementation, there was a significant increase in the relative abundances of probiotics detected in the gut microbiomes. Out of subjects with 3 timepoints, 91% had all three of the common probiotic species we investigated. These results indicate that probiotic supplementation may be changing microbial community composition and function that may alleviate IBS symptoms.

In the microbiome composition of individuals with IBS, there was a significant reduction in alpha diversity and anti-inflammatory microbes while there was an increase in inflammatory microbes. A reduction in alpha diversity in the microbiome may indicate a loss of microbial species in response to different environmental factors (i.e. antibiotics) or a presence of microbial players that may be driving the reduction in diversity. For example, in small intestinal bacterial overgrowth, there was increased abundance in Klebsiella and Escherichia/Shigella and a reduced duodenal microbiome diversity. Consistent with IBS-A, IBS-C, and Crohn's disease studies, anti-inflammatory F. prasunitzii was detected at lower relative abundances in individuals with IBS compared to the healthy cohort. In contrast to previous amplicon-based studies that did not find a reduced abundance of F. prausnitzii in IBS-D, F. prasunitzii was detected at lower levels in IBS-D in this present study. F. prausnitzii enhances gut barrier protection and produces butyrate, a short chain fatty acid that has an important role in gut health. Roseburia intestinalis has an anti-inflammatory role in the gut and is reduced in individuals with Crohn's disease. R. intestinalis was significantly reduced in IBS-C and IBS-D subtype (FIG. 2). Shigella spp., a major contributor to diarrheal disease and associated with post-infectious IBS, was found to be increased in the IBS subtypes (FIG. 2). These variations in the microbial signature was taken into account for the internal health index score. The scoring system is highly dependent on the microbial abundance levels in the profile and their association with gastrointestinal conditions like IBS, arthritis, proper balance of the gut ecosystem including the presence of Faecalibacterium.

The other differentially abundant microbes have an unclear role in IBS. Ruminococcus lactaris is negatively correlated to IL-8 and is more abundant in a non-chronic kidney disease cohort, but has also been shown to be associated with a high-fat diet in a murine diabetes model. Eubacterium rectale is a butyrate producer associated with infant gut microbiome development, but is also associated with obesity and dysbiosis. In a recent metagenomic assembly study of E. rectale, there were different subspecies as a result of genetic and geographic dispersal in human populations, revealing differences in subspecies physiologies and metabolisms. Prevotella spp. are common in non-western plant-rich diets and decreased in individuals with constipation, but have also been associated with chronic inflammatory conditions. These studies indicate that the role of some microbes detected in this study are context and environmentally dependent.

Functional analysis identified pathways associated with each of the phenotypic classifications of IBS. The methanogenesis from acetate pathway was associated with IBS-C (FIG. 2). Methanogenesis contributes to methane production, which is correlated to the severity of constipation and has been used as a diagnostic indicator of constipation predominant IBS. Surprisingly, methanogenesis was also associated with IBS-D. Previous studies have demonstrated the reduction of methanogens in IBS-D. However, Blautia spp. and Fusicatenibacter were microbes detected to have genes that contribute to the methanogenesis pathway (Table 3). Blautia spp. and Fusicatenibacter are known to produce short chain fatty acids and gases through carbohydrate fermentation, substrates for methanogenesis. An overabundance of methanogenesis may lead to gut symptoms in IBS. The Bifidobacterium shunt was also associated with IBS-C. The Bifidobacterium shunt, also called the fructose-6-phosphate shunt, is involved in producing short chain fatty acids (SCFA) and other organic compounds. Depending on the chemical and microbial microenvironment, SCFA can regulate growth and virulence of enteric pathogens. In addition, SCFA stimulate water adsorption in the colon. If too much water is absorbed, stool becomes more solid, resulting in constipation. Factors affecting host physiology in IBS may be dependent upon the microenvironments and microbes present in the gut.

TABLE 3
Biological Pathways and Microbes in IBS.

Pathway - Healthy (Pathway: microbe)
ARGININE-SYN4-PWY: L-ornithine biosynthesis II
Bacteroides_coprocola, Bacteroides_dorei, Bacteroides_eggerthii, Bacteroides_fragilis, Bacteroides_intestinalis,
Bacteroides_ovatus, Bacteroides_thetaiotaomicron, Bacteroides_uniformis, Bacteroides_vulgatus,
Bacteroides_xylanisolvens, Paraprevotella_xylaniphila, Bacteroides_cellulosilyticus, Bacteroides_faecis,
Bacteroides_faecis_CAG_32, Bacteroides_plebeius, Bacteroides_sp_D2, Bacteroides_caccae,
Bacteroides_finegoldii, Bacteroides_massiliensis, Bacteroides_stercoris, Bacteroides_sp_CAG_530,
Catenibacterium_mitsuokai, Bacteroides_clarus, Paraprevotella_clara, Bacteroides_salyersiae,
Bacteroides_nordii, Bacteroides_sp_OM08_11, Bacteroides_stercorirosoris, Bacteroides_fluxus,
Bacteroides_sartorii, Bacteroides_coprophilus, Bacteroides_sp_CAG_443
ECASYN-PWY: enterobacterial common antigen biosynthesis
Escherichia_coli, Citrobacter_braakii, Klebsiella_variicola
GLUCARDEG-PWY: D-glucarate degradation I
Escherichia_coli, Raoultella_planticola, Clostridium_aldenense, Clostridium_citroniae, Klebsiella_pneumoniae,
Klebsiella_variicola
GLUCUROCAT-PWY: superpathway of &beta;-D-glucuronosides degradation
Bacteroides_fragilis, Faecalibacterium_prausnitzii, Escherichia_coli, Hungatella_hathewayi,
Intestinimonas_butyriciproducens, Citrobacter_braakii
GLYCOLYSIS: glycolysis I (from glucose 6-phosphate)
Bacteroides_fragilis, Parabacteroides_distasonis, Bacteroides_cellulosilyticus, Escherichia_coli,
Catenibacterium_mitsuokai, Bacteroides_salyersiae, Bacteroides_nordii, Citrobacter_braakii,
Citrobacter_youngae, Haemophilus_parainfluenzae, Klebsiella_variicola
GOLPDLCAT-PWY: superpathway of glycerol degradation to 1,3-propanediol
Anaerostipes_hadrus, Ruminococcus_torques, Eubacterium_hallii, Flavonifractor_plautii, Citrobacter_braakii,
Klebsiella_pneumoniae, Klebsiella_variicola
HISTSYN-PWY: L-histidine biosynthesis
Agathobaculum_butyriciproducens, Alistipes_finegoldii, Alistipes_shahii, Bacteroides_coprocola,
Bacteroides_dorei, Bacteroides_eggerthii, Bacteroides_fragilis, Bacteroides_intestinalis, Bacteroides_ovatus,
Bacteroides_thetaiotaomicron, Bacteroides_uniformis, Bacteroides_vulgatus, Bacteroides_xylanisolvens,
Barnesiella_intestinihominis, Bifidobacterium_adolescentis, Blautia_obeum, Ruminococcus_torques,
Collinsella_aerofaciens, Faecalibacterium_prausnitzii, Lawsonibacter_asaccharolyticus,
Odoribacter_splanchnicus, Parabacteroides_distasonis, Parabacteroides_merdae, Paraprevotella_xylaniphila,
Ruminococcus_bromii, Streptococcus_thermophilus, Coprobacter_fastidiosus, Eubacterium_ventriosum,
Roseburia_inulinivorans, Bacteroides_caccae, Bacteroides_cellulosilyticus, Bacteroides_plebeius,
Bacteroides_sp_D2, Bifidobacterium_bifidum, Comamonas_kerstersii, Dorea_formicigenerans,
Eubacterium_sp_CAG_180, Eubacterium_sp_CAG_38, Parasutterella_excrementihominis,
Eubacterium_siraeum, Bacteroides_finegoldii, Bacteroides_massiliensis, Bacteroides_sp_CAG_144,
Bacteroides_stercoris, Bifidobacterium_longum, Butyricimonas_synergistica, Butyricimonas_virosa,
Coprococcus_catus, Intestinimonas_butyriciproducens, Methanobrevibacter_smithii, Odoribacter_laneus,
Bacteroides_sp_CAG_530, Catenibacterium_mitsuokai, Eubacterium_sp_CAG_251, Prevotella_sp_CAG_279,
Prevotella_sp_CAG_617, Roseburia_hominis, Slackia_isoflavoniconvertens, Bacteroides_faecis,
Bifidobacterium_pseudocatenulatum, Dialister_sp_CAG_357, Eubacterium_sp_CAG_274,
Megamonas_funiformis, Anaerotruncus_sp_CAG_528, Bacteroides_faecis_CAG_32, Roseburia_intestinalis,
Ruminococcus_sp_CAG_330, Bacteroides_clarus, Paraprevotella_clara, Proteobacteria_bacterium_CAG_139,
Escherichia_coli, Bacteroides_salyersiae, Bacteroides_sp_OM08_11, Ruminococcaceae_bacterium_D16,
Ruminococcus_champanellensis, Coprobacter_sp, Parabacteroides_johnsonii, Bifidobacterium_catenulatum,
Streptococcus_salivarius, Veillonella_atypica, Lactococcus_lactis, Clostridium_leptum, Flavonifractor_plautii,
Alistipes_timonensis, Bacteroides_nordii, Oxalobacter_formigenes, Parabacteroides_goldsteinii,
Coprococcus_eutactus, Alistipes_indistinctus, Ruthenibacterium_lactatiformans, Bacteroides_stercorirosoris,
Clostridium_scindens, Coprobacillus_cateniformis, Clostridium_spiroforme, Lactobacillus_plantarum,
Bacteroides_fluxus, Campylobacter_hominis, Dorea_longicatena, Citrobacter_braakii, Eggerthella_lenta,
Hungatella_hathewayi, Clostridium_bolteae, Firmicutes_bacterium_CAG_94, Bacteroides_sartorii,
Prevotella_copri, Anaerotignum_lactatifermentans, Collinsella_stercoris, Erysipelatoclostridium_ramosum,
Clostridium_citroniae, Clostridium_clostridioforme, Alistipes_onderdonkii, Bacteroides_coprophilus,
Clostridium_disporicum, Bifidobacterium_angulatum, Clostridium_sp_CAG_964,
Parabacteroides_sp_CAG_409, Bacteroides_sp_CAG_443, Prevotella_sp_CAG_5226,
Prevotella_sp_CAG_1185, Bifidobacterium_breve, Cloacibacillus_evryensis, Klebsiella_pneumoniae,
Klebsiella_variicola, Dialister_succinatiphilus, Bacteroides_sp_CAG_633
KETOGLUCONMET-PWY: ketogluconate metabolism
Escherichia_coli, Lactobacillus_plantarum, Enterobacter_cloacae_complex, Citrobacter_braakii,
Klebsiella_variicola
METH-ACETATE-PWY: methanogenesis from acetate
Blautia_obeum, Blautia_wexlerae, Ruminococcus_torques, Fusicatenibacter_saccharivorans,
Blautia_sp_CAG_257
GLIPASYN-PWY: lipid IVA biosynthesis (E. coli)
Bacteroides_uniformis, Bacteroides_cellulosilyticus, Bacteroides_intestinalis, Comamonas_kerstersii,
Escherichia_coli, Parasutterella_excrementihominis, Bacteroides_stercoris, Catenibacterium_mitsuokai,
Proteobacteria_bacterium_CAG_139, Bacteroides_fragilis, Citrobacter_braakii, Haemophilus_parainfluenzae,
Alistipes_sp_CHKCI003, Oxalobacter_formigenes, Klebsiella_variicola, Bacteroides_sp_CAG_633,
Rikenella_microfusus
P124-PWY: Bifidobacterium shunt
Bifidobacterium_longum, Bifidobacterium_pseudocatenulatum, Collinsella_aerofaciens
P161-PWY: acetylene degradation (anaerobic)
Escherichia_coli, Enterobacter_cloacae_complex, Kluyvera_ascorbata, Raoultella_planticola,
Flavonifractor_plautii, Lactococcus_lactis, Dorea_longicatena, Clostridium_aldenense, Citrobacter_braakii,
Citrobacter_youngae, Pseudoflavonifractor_capillosus, Clostridium_citroniae, Klebsiella_variicola
P221-PWY: octane oxidation
Escherichia_coli
PENTOSE-P-PWY: pentose phosphate pathway
Bacteroides_fragilis, Parabacteroides_distasonis, Bacteroides_thetaiotaomicron, Bacteroides_xylanisolvens,
Bifidobacterium_bifidum, Escherichia_coli, Bacteroides_cellulosilyticus, Haemophilus_parainfluenzae,
Dorea_longicatena, Clostridium_aldenense, Lactobacillus_plantarum, Citrobacter_braakii, Clostridium_citroniae,
Haemophilus_sp_HMSC71H05
PHOSLIPSYN-PWY: superpathway of phospholipid biosynthesis I (bacteria)
Fusicatenibacter_saccharivorans, Escherichia_coli, Catenibacterium_mitsuokai, Ruminococcus_gnavus,
Clostridium_leptum, Haemophilus_parainfluenzae, Campylobacter_hominis, Citrobacter_braakii,
Klebsiella_variicola
POLYISOPRENSYN-PWY: polyisoprenoid biosynthesis (E. coli)
Escherichia_coli, Citrobacter_braakii, Klebsiella_variicola
PWY-4321: L-glutamate degradation IV
Escherichia_coli
PWY-5030: L-histidine degradation III
Alistipes_finegoldii, Alistipes_onderdonkii, Alistipes_putredinis, Alistipes_shahii, Bacteroides_dorei,
Bacteroides_eggerthii, Bacteroides_fragilis, Bacteroides_ovatus, Bacteroides_thetaiotaomicron,
Bacteroides_uniformis, Bacteroides_vulgatus, Bacteroides_xylanisolvens, Bacteroides_faecis,
Bacteroides_sp_D2, Bacteroides_stercoris, Bacteroides_massiliensis, Catenibacterium_mitsuokai,
Bacteroides_clarus, Bacteroides_sp_OM08_11, Streptococcus_parasanguinis, Alistipes_timonensis,
Bacteroides_fluxus, Porphyromonas_asaccharolytica, Prevotella_buccalis, Peptoniphilus_sp_HMSC062D09,
Bacteroides_sartorii
PWY-5083: D(P)/DPH interconversion
Escherichia_coli, Enterobacter_cloacae_complex, Kluyvera_ascorbata, Raoultella_planticola,
Citrobacter_braakii, Klebsiella_variicola
PWY-5104: L-isoleucine biosynthesis IV
Parabacteroides_distasonis, Parabacteroides_merdae, Escherichia_coli, Kluyvera_ascorbata,
Parabacteroides_goldsteinii, Eggerthella_lenta, Dorea_longicatena, Citrobacter_braakii
PWY-5189: tetrapyrrole biosynthesis II (from glycine)
Escherichia_coli, Intestinimonas_butyriciproducens, Campylobacter_hominis
PWY-5345: superpathway of L-methionine biosynthesis (by sulfhydrylation)
PWY-5484: glycolysis II (from fructose 6-phosphate)
Bacteroides_fragilis, Parabacteroides_distasonis, Bacteroides_cellulosilyticus, Escherichia_coli,
Catenibacterium_mitsuokai, Bacteroides_salyersiae, Bacteroides_nordii, Citrobacter_braakii,
Citrobacter_youngae, Haemophilus_parainfluenzae, Klebsiella_variicola
PWY-5675: nitrate reduction V (assimilatory)
Escherichia_coli, Enterobacter_cloacae_complex, Kluyvera_ascorbata, Raoultella_planticola,
Klebsiella_variicola
PWY-5695: inosine 5′-phosphate degradation
Agathobaculum_butyriciproducens, Alistipes_finegoldii, Alistipes_inops, Alistipes_onderdonkii,
Alistipes_putredinis, Alistipes_shahii, Anaerostipes_hadrus, Bacteroides_coprocola, Bacteroides_dorei,
Bacteroides_eggerthii, Bacteroides_fragilis, Bacteroides_ovatus, Bacteroides_thetaiotaomicron,
Bacteroides_uniformis, Bacteroides_vulgatus, Bacteroides_xylanisolvens, Barnesiella_intestinihominis,
Blautia_obeum, Blautia_wexlerae, Ruminococcus_torques, Coprococcus_catus, Coprococcus_comes,
Dialister_invisus, Dorea_longicatena, Eubacterium_eligens, Eubacterium_ventriosum,
Faecalibacterium_prausnitzii, Fusicatenibacter_saccharivorans, Lachnospira_pectinoschiza,
Odoribacter_splanchnicus, Parabacteroides_distasonis, Ruminococcus_bicirculans, Roseburia_inulinivorans,
Bacteroides_cellulosilyticus, Bacteroides_faecis, Bacteroides_intestinalis, Bacteroides_massiliensis,
Bacteroides_plebeius, Bacteroides_sp_D2, Bacteroides_stercoris, Comamonas_kerstersii, Coprococcus_eutactus,
Escherichia_coli, Eubacterium_ramulus, Clostridium_clostridioforme, Roseburia_faecis, Bacteroides_caccae,
Bacteroides_finegoldii, Coprobacter_fastidiosus, Firmicutes_bacterium_CAG_94, Flavonifractor_plautii,
Intestinimonas_butyriciproducens, Klebsiella_aerogenes, Lactobacillus_rogosae, Bacteroides_galacturonicus,
Bacteroides_sp_CAG_530, Catenibacterium_mitsuokai, Clostridium_disporicum, Desulfovibrio_piger,
Prevotella_sp_CAG_279, Prevotella_sp_CAG_617, Roseburia_intestinalis, Bacteroides_faecis_CAG_32,
Bacteroides_clarus, Bacteroides_sp_CAG_927, Bilophila_wadsworthia, Hungatella_hathewayi,
Clostridium_bolteae, Alistipes_sp_An31A, Bacteroides_salyersiae, Bacteroides_sp_OM08_11,
Bacteroides_nordii, Ruminococcus_champanellensis, Coprobacter_sp, Eisenbergiella_massiliensis,
Haemophilus_parainfluenzae, Haemophilus_sp_HMSC71H05, Parabacteroides_johnsonii, Romboutsia_ilealis,
Streptococcus_salivarius, Enterobacter_cloacae_complex, Intestinibacter_bartlettii, Kluyvera_ascorbata,
Alistipes_timonensis, Parabacteroides_goldsteinii, Mitsuokella_jalaludinii, Alistipes_indistinctus,
Clostridium_citroniae, Bacteroides_stercorirosoris, Desulfovibrionaceae_bacterium, Clostridium_scindens,
Clostridium_aldenense, Bacteroides_fluxus, Porphyromonas_asaccharolytica, Prevotella_buccalis,
Anaerotruncus_colihominis, Citrobacter_braakii, Bacteroides_sartorii, Prevotella_copri, Collinsella_stercoris,
Bacteroides_coprophilus, Streptococcus_australis, Clostridium_asparagiforme, Alistipes_sp_CHKCI003,
Bacteroides_sp_CAG_443, Bacteroides_sp_43_108, Veillonella_parvula, Klebsiella_pneumoniae,
Desulfovibrio_fairfieldensis, Bacteroides_sp_CAG_633
PWY-5723: Rubisco shunt
Escherichia_coli, Raoultella_planticola, Citrobacter_braakii, Klebsiella_variicola
PWY-5918: superpathway of heme b biosynthesis from glutamate
Escherichia_coli, Citrobacter_braakii
PWY-5971: palmitate biosynthesis (type II fatty acid synthase)
Escherichia_coli, Citrobacter_braakii
PWY-622: starch biosynthesis
PWY-6284: superpathway of unsaturated fatty acids biosynthesis (E. coli)
Escherichia_coli, Citrobacter_braakii
PWY-6507: 4-deoxy-L-threo-hex-4-enopyranuronate degradation Faecalibacterium_prausnitzii,
Lactobacillus_rhamnosus, Escherichia_coli, Hungatella_hathewayi, Clostridium_bolteae, Dorea_longicatena,
Clostridium_citroniae, Clostridium_aldenense, Anaerotruncus_colihominis, Citrobacter_braakii,
Citrobacter_youngae, Clostridium_clostridioforme
PWY-6545: pyrimidine deoxyribonucleotides de novo biosynthesis III
Escherichia_coli
PWY-6803: phosphatidylcholine acyl editing
Escherichia_coli, Citrobacter_braakii, Klebsiella_pneumoniae, Raoultella_planticola, Citrobacter_youngae,
Klebsiella_quasipneumoniae, Klebsiella_variicola
PWY-7184: pyrimidine deoxyribonucleotides de novo biosynthesis I
Escherichia_coli, Citrobacter_braakii, Haemophilus_parainfluenzae, Klebsiella_variicola
PWY-7204: pyridoxal 5′-phosphate salvage II (plants)
Escherichia_coli, Citrobacter_braakii
PWY-7210: pyrimidine deoxyribonucleotides biosynthesis from CTP
Escherichia_coli, Haemophilus_parainfluenzae
PWY-7211: superpathway of pyrimidine deoxyribonucleotides de novo biosynthesis
Escherichia_coli
PWY-7242: D-fructuronate degradation
Bacteroides_fragilis, Faecalibacterium_prausnitzii, Escherichia_coli, Intestinimonas_butyriciproducens,
Hungatella_hathewayi, Clostridium_sp_CAG_58, Lactococcus_lactis, Dorea_longicatena,
Clostridium_aldenense, Pseudoflavonifractor_capillosus, Citrobacter_braakii, Citrobacter_youngae
PWY-7269: mitochondrial DPH production (yeast)
Escherichia_coli, Enterobacter_cloacae_complex, Raoultella_planticola, Klebsiella_pneumoniae,
Klebsiella_variicola
PWY-7282: 4-amino-2-methyl-5-diphosphomethylpyrimidine biosynthesis II
Bacteroides_fragilis, Bacteroides_ovatus, Bacteroides_uniformis, Bacteroides_xylanisolvens,
Bacteroides_sp_D2, Escherichia_coli, Klebsiella_aerogenes, Catenibacterium_mitsuokai, Citrobacter_braakii,
Haemophilus_parainfluenzae, Streptococcus_salivarius
PWY-7392: taxadiene biosynthesis (engineered)
PWY-7446: sulfoquinovose degradation I
Escherichia_coli, Citrobacter_braakii
PWY-7456: &beta;-(1,4)-mannan degradation
Coprococcus_eutactus, Eubacterium_siraeum, Roseburia_faecis, Clostridium_disporicum
PWY0-1298: superpathway of pyrimidine deoxyribonucleosides degradation
Escherichia_coli, Hungatella_hathewayi, Kluyvera_ascorbata, Raoultella_planticola, Lactococcus_lactis,
Dorea_longicatena, Citrobacter_braakii, Klebsiella_variicola
PWY3O-355: stearate biosynthesis III (fungi)
Bacteroides_dorei, Bacteroides_vulgatus, Bacteroides_uniformis, Bifidobacterium_longum
PWY4FS-7: phosphatidylglycerol biosynthesis I (plastidic)
Faecalibacterium_prausnitzii, Fusicatenibacter_saccharivorans, Streptococcus_thermophilus, Escherichia_coli,
Catenibacterium_mitsuokai, Roseburia_intestinalis, Streptococcus_salivarius, Ruminococcus_gnavus,
Haemophilus_parainfluenzae, Clostridium_leptum, Campylobacter_hominis, Citrobacter_braakii,
Klebsiella_variicola
PWY4FS-8: phosphatidylglycerol biosynthesis II (non-plastidic)
Faecalibacterium_prausnitzii, Fusicatenibacter_saccharivorans, Streptococcus_thermophilus, Escherichia_coli,
Catenibacterium_mitsuokai, Roseburia_intestinalis, Streptococcus_salivarius, Ruminococcus_gnavus,
Haemophilus_parainfluenzae, Clostridium_leptum, Campylobacter_hominis, Citrobacter_braakii,
Klebsiella_variicola
PWY4LZ-257: superpathway of fermentation (Chlamydomonas reinhardtii)
Escherichia_coli
PWY66-389: phytol degradation
Escherichia_coli, Clostridium_citroniae, Haemophilus_parainfluenzae
PYRIDNUCSAL-PWY: D salvage pathway I (PNC VI cycle)
Escherichia_coli, Bifidobacterium_catenulatum
RIBOSYN2-PWY: flavin biosynthesis I (bacteria and plants)
Agathobaculum_butyriciproducens, Akkermansia_muciniphila, Anaerostipes_hadrus, Bacteroides_coprocola,
Bacteroides_dorei, Bacteroides_eggerthii, Bacteroides_fragilis, Bacteroides_ovatus,
Bacteroides_thetaiotaomicron, Bacteroides_uniformis, Bacteroides_vulgatus, Bacteroides_xylanisolvens,
Barnesiella_intestinihominis, Blautia_obeum, Blautia_wexlerae, Ruminococcus_torques,
Coprobacillus_cateniformis, Coprococcus_comes, Dorea_longicatena, Eubacterium_eligens, Eubacterium_hallii,
Faecalibacterium_prausnitzii, Lachnospira_pectinoschiza, Eubacterium_rectale, Odoribacter_splanchnicus,
Parabacteroides_distasonis, Parabacteroides_merdae, Paraprevotella_xylaniphila, Roseburia_hominis,
Coprobacter_fastidiosus, Bacteroides_cellulosilyticus, Bacteroides_intestinalis, Bacteroides_sp_D2,
Clostridium_sp_CAG_167, Coprococcus_eutactus, Dorea_formicigenerans, Eubacterium_ramulus,
Eubacterium_sp_CAG_38, Phascolarctobacterium_sp_CAG_266, Ruminococcus_callidus,
Sanguibacteroides_justesenii, Adlercreutzia_equolifaciens, Asaccharobacter_celatus, Bacteroides_caccae,
Bacteroides_finegoldii, Bacteroides_massiliensis, Bacteroides_stercoris, Bilophila_wadsworthia,
Butyrivibrio_crossotus, Coprococcus_catus, Escherichia_coli, Lactobacillus_rogosae, Odoribacter_laneus,
Phascolarctobacterium_faecium, Bacteroides_galacturonicus, Bacteroides_plebeius, Desulfovibrio_piger,
Phascolarctobacterium_succinatutens, Roseburia_faecis, Victivallis_vadensis, Bacteroides_faecis,
Clostridium_sp_CAG_411, Dialister_sp_CAG_357, Megamonas_funiformis, Megamonas_hypermegale,
Brachyspira_sp_CAG_700, Roseburia_intestinalis, Catenibacterium_mitsuokai, Ruminococcus_sp_CAG_330,
Bacteroides_clarus, Bacteroides_sp_CAG_927, Paraprevotella_clara, Prevotella_copri, Bacteroides_salyersiae,
Bacteroides_sp_OM08_11, Anaerotignum_lactatifermentans, Desulfovibrio_fairfieldensis,
Ruminococcaceae_bacterium_D16, Coprobacter_sp, Parabacteroides_johnsonii, Clostridium_sp_CAG_253,
Allisonella_histaminiformans, Holdemanella_biformis, Intestinibacter_bartlettii, Lactococcus_lactis,
Ruminococcus_sp_CAG_403, Slackia_isoflavoniconvertens, Acidaminococcus_intestini, Lactobacillus_reuteri,
Parabacteroides_goldsteinii, Bacteroides_stercorirosoris, Clostridium_aldenense, Clostridium_sp_CAG_299,
Campylobacter_hominis, Porphyromonas_asaccharolytica, Prevotella_buccalis, Roseburia_sp_CAG_303,
Citrobacter_braakii, Hungatella_hathewayi, Clostridium_bolteae, Clostridium_clostridioforme,
Flavonifractor_plautii, Bifidobacterium_longum, Bacteroides_sartorii, Clostridium_citroniae,
Prevotella_sp_CAG_279, Prevotella_corporis, Bacteroides_coprophilus, Clostridium_disporicum,
Haemophilus_parainfluenzae, Eubacterium_sp_CAG_274, Clostridium_sp_CAG_964, Bacteroides_nordii,
Bacteroides_sp_CAG_443, Parabacteroides_sp_CAG_409, Prevotella_sp_CAG_1185, Veillonella_atypica,
Veillonella_parvula, Bacteroides_sp_43_108, Clostridium_spiroforme, Desulfovibrionaceae_bacterium,
Klebsiella_variicola, Dialister_succinatiphilus
SO4ASSIM-PWY: assimilatory sulfate reduction I
Escherichia_coli, Klebsiella_aerogenes, Klebsiella_pneumoniae, Enterobacter_cloacae_complex,
Kluyvera_ascorbata, Raoultella_planticola, Haemophilus_parainfluenzae, Citrobacter_braakii,
Odoribacter_laneus
SULFATE-CYS-PWY: superpathway of sulfate assimilation and cysteine biosynthesis
Escherichia_coli, Klebsiella_aerogenes, Enterobacter_cloacae_complex, Raoultella_planticola,
Citrobacter_braakii
TCA: TCA cycle I (prokaryotic)
Escherichia_coli, Comamonas_kerstersii, Campylobacter_hominis, Citrobacter_braakii, Citrobacter_youngae,
Klebsiella_variicola
TEICHOICACID-PWY: poly(glycerol phosphate) wall teichoic acid biosynthesis
Bacteroides_cellulosilyticus, Bacteroides_vulgatus, Ruminococcus_torques, Parabacteroides_distasonis,
Bacteroides_thetaiotaomicron, Lactobacillus_plantarum, Bacteroides_uniformis
THISYRA-PWY: superpathway of thiamine diphosphate biosynthesis III (eukaryotes)
Bacteroides_fragilis, Bacteroides_ovatus, Bacteroides_uniformis, Ruminococcus_torques, Dorea_longicatena,
Faecalibacterium_prausnitzii, Eubacterium_rectale, Bacteroides_xylanisolvens, Roseburia_hominis,
Bifidobacterium_bifidum, Eubacterium_hallii, Roseburia_faecis, Butyrivibrio_crossotus, Bacteroides_plebeius,
Catenibacterium_mitsuokai, Holdemanella_biformis, Phascolarctobacterium_succinatutens,
Roseburia_intestinalis, Bacteroides_sp_OM08_11, Ruminococcus_gnavus, Coprococcus_eutactus,
Clostridium_scindens, Clostridiales_bacterium_1_7_47FAA, Gordonibacter_pamelaeae, Clostridium_aldenense,
Clostridium_citroniae, Bifidobacterium_angulatum
Pathway - IBS-A (Pathway: microbe)
ARGININE-SYN4-PWY: L-ornithine biosynthesis II
Bacteroides_vulgatus, Bacteroides_caccae, Bacteroides_faecis, Bacteroides_faecis_CAG_32,
Bacteroides_finegoldii, Bacteroides_massiliensis, Bacteroides_stercoris, Bacteroides_thetaiotaomicron,
Bacteroides_uniformis, Bacteroides_intestinalis, Bacteroides_ovatus, Bacteroides_salyersiae,
Bacteroides_cellulosilyticus, Bacteroides_fragilis, Bacteroides_xylanisolvens, Paraprevotella_xylaniphila,
Bacteroides_dorei, Bacteroides_coprocola, Bacteroides_plebeius, Paraprevotella_clara, Bacteroides_eggerthii,
Catenibacterium_mitsuokai, Klebsiella_pneumoniae, Bacteroides_clarus, Bacteroides_coprophilus,
Citrobacter_freundii, Bacteroides_sp_D2, Bacteroides_sartorii, Bacteroides_nordii, Bacteroides_sp_OM05_12,
Bacteroides_sp_OM08_11, Bacteroides_fluxus
ECASYN-PWY: enterobacterial common antigen biosynthesis
Escherichia_coli, Klebsiella_pneumoniae, Enterobacter_cloacae_complex, Klebsiella_variicola,
Citrobacter_freundii, Enterobacter_bugandensis, Serratia_marcescens, Citrobacter_youngae,
Citrobacter_amalonaticus, Citrobacter_portucalensis, Kluyvera_ascorbata, Citrobacter_braakii,
Klebsiella_oxytoca
GLUCARDEG-PWY: D-glucarate degradation I
Escherichia_coli, Klebsiella_pneumoniae, Clostridium_aldenense, Klebsiella_variicola, Raoultella_planticola,
Clostridium_citroniae, Clostridium_clostridioforme, Enterobacter_cloacae_complex, Citrobacter_freundii,
Enterobacter_bugandensis, Citrobacter_youngae, Citrobacter_amalonaticus, Hungatella_hathewayi,
Citrobacter_portucalensis, Kluyvera_ascorbata, Citrobacter_braakii, Leclercia_adecarboxylata
GLUCUROCAT-PWY: superpathway of &beta;- D-glucuronosides degradation
Escherichia_coli, Faecalibacterium_prausnitzii, Hungatella_hathewayi, Klebsiella_pneumoniae,
Bacteroides_fragilis, Intestinimonas_butyriciproducens, Citrobacter_freundii, Citrobacter_youngae,
Lactobacillus_rhamnosus, Citrobacter_portucalensis, Kluyvera_ascorbata, Citrobacter_braakii,
Enterobacter_cloacae_complex
GLYCOLYSIS: glycolysis I (from glucose 6-phosphate)
Escherichia_coli, Hafnia_alvei, Klebsiella_pneumoniae, Citrobacter_freundii, Klebsiella_variicola,
Raoultella_ornithinolytica, Raoultella_planticola, Bacteroides_cellulosilyticus, Bacteroides_fragilis,
Bacteroides_salyersiae, Enterobacter_cloacae_complex, Haemophilus_parainfluenzae,
Haemophilus_sp_HMSC71H05, Citrobacter_youngae, Klebsiella_michiganensis, Serratia_marcescens,
Lactobacillus_delbrueckii, Citrobacter_amalonaticus, Citrobacter_portucalensis, Kluyvera_ascorbata,
Catenibacterium_mitsuokai, Bacteroides_nordii, Citrobacter_braakii
GOLPDLCAT-PWY: superpathway of glycerol degradation to 1,3-propanediol
Ruminococcus_torques, Anaerostipes_hadrus, Flavonifractor_plautii, Citrobacter_braakii, Escherichia_coli,
Klebsiella_pneumoniae, Klebsiella_variicola, Citrobacter_freundii, Hafnia_paralvei, Klebsiella_michiganensis,
Citrobacter_youngae, Citrobacter_portucalensis, Enterobacter_cloacae_complex, Klebsiella_oxytoca
HISTSYN-PWY: L-histidine biosynthesis
Bacteroides_dorei, Bacteroides_massiliensis, Bacteroides_vulgatus, Ruminococcus_torques,
Dialister_succinatiphilus, Escherichia_coli, Hafnia_alvei, Klebsiella_pneumoniae, Megamonas_funiformis,
Parabacteroides_distasonis, Parabacteroides_merdae, Bacteroides_caccae, Bacteroides_faecis_CAG_32,
Bacteroides_finegoldii, Bacteroides_fluxus, Bacteroides_salyersiae, Bacteroides_stercoris,
Bacteroides_uniformis, Bacteroides_xylanisolvens, Blautia_obeum, Collinsella_aerofaciens,
Faecalibacterium_prausnitzii, Megasphaera_sp_DISK_18, Ruminococcus_bromii, Alistipes_shahii,
Bacteroides_intestinalis, Bacteroides_thetaiotaomicron, Butyricimonas_synergistica, Eisenbergiella_massiliensis,
Odoribacter_splanchnicus, Bacteroides_cellulosilyticus, Bacteroides_ovatus, Clostridium_bolteae_CAG_59,
Erysipelatoclostridium_ramosum, Flavonifractor_plautii, Hungatella_hathewayi, Clostridium_bolteae,
Parasutterella_excrementihominis, Roseburia_intestinalis, Roseburia_inulinivorans,
Ruthenibacterium_lactatiformans, Klebsiella_variicola, Pseudomonas_aeruginosa_group,
Raoultella_ornithinolytica, Raoultella_planticola, Alistipes_onderdonkii, Bacteroides_fragilis,
Eubacterium_sp_CAG_180, Paraprevotella_xylaniphila, Alistipes_finegoldii, Clostridium_aldenense,
Clostridium_clostridioforme, Clostridium_scindens, Barnesiella_intestinihominis, Bacteroides_faecis,
Bacteroides_nordii, Eubacterium_siraeum, Candidatus_Stoquefichus_sp_KLE1796, Eubacterium_sp_CAG_274,
Proteobacteria_bacterium_CAG_139, Bacteroides_coprocola, Bacteroides_plebeius, Butyricimonas_virosa,
Coprococcus_catus, Dorea_formicigenerans, Clostridium_spiroforme, Paraprevotella_clara,
Bifidobacterium_adolescentis, Eubacterium_ventriosum, Eisenbergiella_tayi, Clostridium_lavalense,
Bacteroides_eggerthii, Catenibacterium_mitsuokai, Agathobaculum_butyriciproducens,
Bifidobacterium_longum, Eubacterium_sp_CAG_38, Collinsella_stercoris, Clostridium_innocuum,
Anaeromassilibacillus_sp_An250, Streptococcus_salivarius, Absiella_dolichum,
Eubacterium_dolichum_CAG_375, Veillonella_parvula, Intestinimonas_butyriciproducens,
Eubacterium_sp_CAG_251, Parabacteroides_johnsonii, Clostridium_leptum, Klebsiella_quasipneumoniae,
Anaerotignum_lactatifermentans, Bacteroides_coprophilus, Dialister_sp_CAG_357, Citrobacter_freundii,
Citrobacter_youngae, Prevotella_copri, Prevotella_sp_AM42_24, Pseudomonas_fragi, Coprobacter_fastidiosus,
Bacteroides_clarus, Clostridiales_bacterium_1_7_47FAA, Enterobacter_bugandensis,
Enterobacter_cloacae_complex, Serratia_marcescens, Streptococcus_thermophilus, Anaerostipes_caccae,
Eggerthella_lenta, Bifidobacterium_bifidum, Odoribacter_laneus, Bacteroides_sp_D2,
Megasphaera_sp_MJR8396C, Citrobacter_amalonaticus, Coprobacter_sp, Bifidobacterium_pseudocatenulatum,
Slackia_isoflavoniconvertens, Streptococcus_pasteurianus, Veillonella_atypica, Oxalobacter_formigenes,
Lawsonibacter_asaccharolyticus, Firmicutes_bacterium_CAG_424, Clostridium_asparagiforme,
Haemophilus_parainfluenzae, Roseburia_hominis, Bacteroides_sartorii, Pseudoflavonifractor_sp_An184,
Acidaminococcus_fermentans, Prevotella_sp_CAG_279, Alistipes_indistinctus, Parabacteroides_sp_CAG_409,
Firmicutes_bacterium_CAG_94, Methanobrevibacter_smithii, Bacteroides_sp_CAG_144, Megasphaera_elsdenii,
Pseudomonas_helleri, Pseudomonas_lundensis, Pseudomonas_psychrophila, Pseudomonas_taetrolens,
Bacteroides_sp_OM05_12, Bacteroides_sp_OM08_11, Parabacteroides_goldsteinii, Citrobacter_braakii,
Klebsiella_oxytoca, Ruminococcaceae_bacterium_D16
KETOGLUCONMET-PWY: ketogluconate metabolism
Escherichia_coli, Klebsiella_pneumoniae, Klebsiella_variicola, Pseudomonas_aeruginosa_group,
Enterobacter_cloacae_complex, Citrobacter_freundii, Serratia_marcescens, Citrobacter_youngae,
Enterococcus_faecium, Lactobacillus_plantarum, Pseudomonas_lundensis, Citrobacter_braakii,
Klebsiella_oxytoca
METH-ACETATE-PWY: methanogenesis from acetate
Blautia_obeum, Fusicatenibacter_saccharivorans, Blautia_sp_CAG_257, Blautia_wexlerae,
Ruminococcus_torques, Blautia_hydrogenotrophica
GLIPASYN-PWY: lipid IVA biosynthesis (E. coli)
Escherichia_coli, Hafnia_alvei, Bacteroides_stercoris, Bacteroides_uniformis, Bacteroides_intestinalis,
Parasutterella_excrementihominis, Klebsiella_pneumoniae, Klebsiella_variicola,
Pseudomonas_aeruginosa_group, Raoultella_ornithinolytica, Raoultella_planticola, Bacteroides_cellulosilyticus,
Proteobacteria_bacterium_CAG_139, Enterobacter_cloacae_complex, Haemophilus_parainfluenzae,
Haemophilus_sp_HMSC71H05, Catenibacterium_mitsuokai, Citrobacter_freundii, Pseudomonas_fragi,
Bacteroides_fragilis, Enterobacter_bugandensis, Serratia_marcescens, Citrobacter_youngae,
Citrobacter_amalonaticus, Citrobacter_portucalensis, Kluyvera_ascorbata, Turicimonas_muris,
Pseudomonas_lundensis, Pseudomonas_psychrophila, Pseudomonas_taetrolens, Citrobacter_braakii,
Klebsiella_oxytoca
P124-PWY: Bifidobacterium shunt
Collinsella_aerofaciens, Bifidobacterium_longum, Bifidobacterium_pseudocatenulatum,
Bifidobacterium_dentium
P161-PWY: acetylene degradation (anaerobic)
Escherichia_coli, Hafnia_alvei, Klebsiella_pneumoniae, Citrobacter_freundii, Flavonifractor_plautii,
Citrobacter_braakii, Klebsiella_variicola, Kluyvera_cryocrescens, Raoultella_ornithinolytica,
Raoultella_planticola, Hungatella_hathewayi, Clostridium_aldenense, Clostridium_citroniae,
Enterobacter_cloacae_complex, Streptococcus_australis, Streptococcus_parasanguinis, Streptococcus_infantis,
Citrobacter_youngae, Klebsiella_quasipneumoniae, Clostridiales_bacterium_1_7_47FAA,
Klebsiella_michiganensis, Serratia_marcescens, Citrobacter_amalonaticus, Citrobacter_portucalensis,
Kluyvera_ascorbata, Leclercia_adecarboxylata
P221-PWY: octane oxidation
Escherichia_coli, Hafnia_alvei, Pseudomonas_aeruginosa_group, Raoultella_ornithinolytica,
Raoultella_planticola, Klebsiella_pneumoniae, Kluyvera_ascorbata, Enterobacter_cloacae_complex
PENTOSE-P-PWY: pentose phosphate pathway
Escherichia_coli, Hafnia_alvei, Parabacteroides_distasonis, Bacteroides_thetaiotaomicron,
Clostridium_aldenense, Klebsiella_pneumoniae, Klebsiella_variicola, Raoultella_ornithinolytica,
Bacteroides_cellulosilyticus, Hungatella_hathewayi, Clostridium_clostridioforme,
Enterobacter_cloacae_complex, Bacteroides_fragilis, Bacteroides_xylanisolvens, Haemophilus_parainfluenzae,
Haemophilus_sp_HMSC71H05, Clostridium_citroniae, Citrobacter_freundii,
Clostridiales_bacterium_1_7_47FAA, Enterobacter_bugandensis, Klebsiella_michiganensis,
Serratia_marcescens, Citrobacter_youngae, Lactobacillus_rhamnosus, Bifidobacterium_bifidum,
Citrobacter_amalonaticus, Citrobacter_portucalensis, Citrobacter_braakii, Leclercia_adecarboxylata
PHOSLIPSYN-PWY: superpathway of phospholipid biosynthesis I (bacteria)
Escherichia_coli, Hafnia_alvei, Fusicatenibacter_saccharivorans, Ruminococcus_gnavus,
Klebsiella_pneumoniae, Klebsiella_variicola, Pseudomonas_aeruginosa_group, Raoultella_ornithinolytica,
Raoultella_planticola, Enterobacter_cloacae_complex, Haemophilus_parainfluenzae,
Haemophilus_sp_HMSC71H05, Clostridium_leptum, Citrobacter_freundii, Pseudomonas_fragi,
Klebsiella_michiganensis, Serratia_marcescens, Citrobacter_youngae, Citrobacter_amalonaticus,
Citrobacter_portucalensis, Kluyvera_ascorbata, Catenibacterium_mitsuokai, Fusobacterium_mortiferum,
Pseudomonas_lundensis, Pseudomonas_psychrophila, Citrobacter_braakii, Klebsiella_oxytoca
POLYISOPRENSYN-PWY: polyisoprenoid biosynthesis (E. coli)
Escherichia_coli, Klebsiella_pneumoniae, Klebsiella_variicola, Citrobacter_freundii, Hafnia_paralvei,
Citrobacter_youngae, Citrobacter_amalonaticus, Citrobacter_portucalensis, Pseudomonas_lundensis,
Citrobacter_braakii, Enterobacter_cloacae_complex, Klebsiella_oxytoca
PWY-4321: L-glutamate degradation IV
Escherichia_coli, Hafnia_alvei, Klebsiella_pneumoniae
PWY-5030: L-histidine degradation III
Bacteroides_vulgatus, Alistipes_putredinis, Bacteroides_faecis, Bacteroides_fluxus, Bacteroides_massiliensis,
Bacteroides_stercoris, Bacteroides_thetaiotaomicron, Bacteroides_uniformis, Bacteroides_ovatus,
Alistipes_onderdonkii, Bacteroides_fragilis, Bacteroides_dorei, Streptococcus_parasanguinis,
Bacteroides_xylanisolvens, Alistipes_finegoldii, Alistipes_shahii, Streptococcus_australis,
Streptococcus_gordonii, Bacteroides_eggerthii, Catenibacterium_mitsuokai, Bacteroides_clarus,
Clostridium_saccharolyticum, Streptococcus_cristatus, Streptococcus_salivarius, Streptococcus_sanguinis,
Streptococcus_sp_F0442, Bacteroides_sp_D2, Bacteroides_sartorii, Acidaminococcus_fermentans,
Bacteroides_sp_OM05_12, Bacteroides_sp_OM08_11
PWY-5083: D(P)/DPH interconversion
Escherichia_coli, Klebsiella_pneumoniae, Citrobacter_braakii, Citrobacter_freundii, Klebsiella_variicola,
Kluyvera_cryocrescens, Raoultella_ornithinolytica, Raoultella_planticola, Enterobacter_cloacae_complex,
Klebsiella_quasipneumoniae, Serratia_marcescens, Citrobacter_youngae, Citrobacter_portucalensis,
Kluyvera_ascorbata, Leclercia_adecarboxylata
PWY-5104: L-isoleucine biosynthesis IV
Escherichia_coli, Parabacteroides_distasonis, Parabacteroides_merdae, Klebsiella_pneumoniae,
Pseudomonas_aeruginosa_group, Eggerthella_lenta, Enterobacter_cloacae_complex, Klebsiella_variicola,
Citrobacter_freundii, Citrobacter_youngae, Citrobacter_portucalensis, Kluyvera_ascorbata,
Parabacteroides_goldsteinii, Citrobacter_braakii, Dorea_longicatena
PWY-5189: tetrapyrrole biosynthesis II (from glycine)
Escherichia_coli, Klebsiella_pneumoniae, Pseudomonas_aeruginosa_group, Enterobacter_cloacae_complex,
Streptococcus_australis, Streptococcus_parasanguinis, Intestinimonas_butyriciproducens, Pseudomonas_fragi,
Serratia_marcescens, Pseudomonas_helleri, Citrobacter_amalonaticus, Pseudomonas_lundensis,
Pseudomonas_psychrophila, Leclercia_adecarboxylata
PWY-5345: superpathway of L-methionine biosynthesis (by sulfhydrylation)
Klebsiella_pneumoniae
PWY-5484: glycolysis II (from fructose 6-phosphate)
Escherichia_coli, Hafnia_alvei, Klebsiella_pneumoniae, Klebsiella_variicola, Raoultella_ornithinolytica,
Raoultella_planticola, Bacteroides_cellulosilyticus, Bacteroides_fragilis, Bacteroides_salyersiae,
Enterobacter_cloacae_complex, Haemophilus_parainfluenzae, Haemophilus_sp_HMSC71H05,
Klebsiella_quasipneumoniae, Citrobacter_freundii, Citrobacter_youngae, Klebsiella_michiganensis,
Serratia_marcescens, Lactobacillus_delbrueckii, Citrobacter_amalonaticus, Citrobacter_portucalensis,
Kluyvera_ascorbata, Catenibacterium_mitsuokai, Bacteroides_nordii, Citrobacter_braakii
PWY-5675: nitrate reduction V (assimilatory)
Escherichia_coli, Klebsiella_pneumoniae, Klebsiella_variicola, Kluyvera_cryocrescens,
Raoultella_ornithinolytica, Raoultella_planticola, Enterobacter_cloacae_complex, Klebsiella_quasipneumoniae,
Citrobacter_freundii, Enterobacter_bugandensis, Serratia_marcescens, Citrobacter_amalonaticus,
Kluyvera_ascorbata, Klebsiella_oxytoca
PWY-5695: inosine 5′-phosphate degradation
Bacteroides_dorei, Bacteroides_massiliensis, Bacteroides_stercoris, Bacteroides_vulgatus,
Bacteroides_xylanisolvens, Ruminococcus_torques, Dorea_longicatena, Escherichia_coli, Eubacterium_ramulus,
Hafnia_alvei, Klebsiella_pneumoniae, Parabacteroides_distasonis, Alistipes_putredinis, Anaerostipes_hadrus,
Bacteroides_caccae, Bacteroides_faecis, Bacteroides_faecis_CAG_32, Bacteroides_finegoldii,
Bacteroides_fluxus, Bacteroides_thetaiotaomicron, Bacteroides_uniformis, Coprococcus_comes,
Fusicatenibacter_saccharivorans, Lachnospira_pectinoschiza, Megasphaera_sp_DISK_18, Roseburia_faecis,
Bacteroides_intestinalis, Bacteroides_ovatus, Bacteroides_salyersiae, Blautia_coccoides, Blautia_obeum,
Blautia_wexlerae, Eisenbergiella_massiliensis, Faecalibacterium_prausnitzii, Odoribacter_splanchnicus,
Roseburia_inulinivorans, Alistipes_shahii, Bacteroides_cellulosilyticus, Bilophila_wadsworthia,
Hungatella_hathewayi, Clostridium_aldenense, Roseburia_intestinalis, Eubacterium_eligens,
Pseudomonas_aeruginosa_group, Raoultella_ornithinolytica, Raoultella_planticola, Ruminococcus_bicirculans,
Agathobaculum_butyriciproducens, Alistipes_onderdonkii, Bacteroides_fragilis, Erwinia_persicina,
Eubacterium_ventriosum, Dialister_invisus, Flavonifractor_plautii, Clostridium_bolteae, Clostridium_citroniae,
Clostridium_clostridioforme, Clostridium_scindens, Clostridium_symbiosum, Alistipes_finegoldii,
Bacteroides_galacturonicus, Barnesiella_intestinihominis, Enterobacter_cloacae_complex,
Lactobacillus_rogosae, Bacteroides_coprocola, Bacteroides_plebeius, Coprococcus_catus,
Streptococcus_sp_A12, Haemophilus_parainfluenzae, Haemophilus_sp_HMSC71H05, Streptococcus_oralis,
Streptococcus_salivarius, Alistipes_inops, Eisenbergiella_tayi, Intestinimonas_butyriciproducens,
Bacteroides_eggerthii, Catenibacterium_mitsuokai, Megasphaera_micronuciformis, Veillonella_parvula,
Klebsiella_oxytoca, Parabacteroides_johnsonii, Klebsiella_variicola, Bacteroides_coprophilus,
Citrobacter_freundii, Hafnia_paralvei, Pseudomonas_fragi, Coprobacter_fastidiosus, Bacteroides_clarus,
Clostridiales_bacterium_1_7_47FAA, Enterobacter_bugandensis, Serratia_marcescens,
Clostridium_saccharolyticum, Citrobacter_youngae, Coprococcus_eutactus, Streptococcus_sanguinis,
Streptococcus_thermophilus, Blautia_hydrogenotrophica, Streptococcus_sp_F0442, Bacteroides_sp_D2,
Megasphaera_sp_MJR8396C, Citrobacter_amalonaticus, Citrobacter_portucalensis, Kluyvera_ascorbata,
Veillonella_atypica, Alistipes_sp_An31A, Bacteroides_sartorii, Alistipes_indistinctus, Prevotella_sp_CAG_279,
Bacteroides_nordii, Prevotella_sp_CAG_1031, Prevotella_sp_AM42_24, Megasphaera_elsdenii,
Parabacteroides_goldsteinii, Pseudomonas_helleri, Pseudomonas_lundensis, Pseudomonas_psychrophila,
Bacteroides_sp_OM05_12, Intestinibacter_bartlettii, Bacteroides_sp_OM08_11, Citrobacter_braakii,
Alistipes_timonensis
PWY-5723: Rubisco shunt
Escherichia_coli, Klebsiella_pneumoniae, Klebsiella_variicola, Enterobacter_cloacae_complex,
Citrobacter_freundii, Klebsiella_michiganensis, Serratia_marcescens, Citrobacter_youngae,
Citrobacter_amalonaticus, Citrobacter_portucalensis, Citrobacter_braakii, Leclercia_adecarboxylata
PWY-5918: superpathway of heme b biosynthesis from glutamate
Escherichia_coli, Klebsiella_pneumoniae, Pseudomonas_aeruginosa_group, Enterobacter_cloacae_complex,
Klebsiella_variicola, Citrobacter_freundii, Pseudomonas_fragi, Serratia_marcescens, Citrobacter_youngae,
Citrobacter_amalonaticus, Citrobacter_portucalensis, Pseudomonas_helleri, Pseudomonas_lundensis,
Pseudomonas_psychrophila, Citrobacter_braakii
PWY-5971: palmitate biosynthesis (type II fatty acid synthase)
Escherichia_coli, Klebsiella_pneumoniae, Enterobacter_cloacae_complex, Citrobacter_freundii,
Citrobacter_youngae, Citrobacter_amalonaticus, Citrobacter_portucalensis, Citrobacter_braakii,
Klebsiella_oxytoca
PWY-622: starch biosynthesis
PWY-6284: superpathway of unsaturated fatty acids biosynthesis (E. coli)
Escherichia_coli, Citrobacter_freundii, Citrobacter_braakii, Klebsiella_pneumoniae,
Enterobacter_cloacae_complex, Citrobacter_youngae, Enterobacter_bugandensis, Citrobacter_amalonaticus,
Citrobacter_portucalensis, Klebsiella_oxytoca
PWY-6507: 4-deoxy-L-threo-hex-4-enopyranuronate degradation
Escherichia_coli, Faecalibacterium_prausnitzii, Hungatella_hathewayi, Clostridium_bolteae,
Klebsiella_pneumoniae, Clostridium_aldenense, Clostridium_citroniae, Clostridium_clostridioforme,
Enterobacter_cloacae_complex, Clostridiales_bacterium_1_7_47FAA, Citrobacter_freundii,
Citrobacter_youngae, Lactobacillus_rhamnosus, Citrobacter_amalonaticus, Citrobacter_portucalensis,
Kluyvera_ascorbata, Citrobacter_braakii, Klebsiella_oxytoca
PWY-6545: pyrimidine deoxyribonucleotides de novo biosynthesis III
Escherichia_coli, Klebsiella_pneumoniae, Serratia_marcescens, Kluyvera_ascorbata,
Enterobacter_cloacae_complex
PWY-6803: phosphatidylcholine acyl editing
Escherichia_coli, Hafnia_alvei, Klebsiella_pneumoniae, Citrobacter_freundii, Citrobacter_youngae,
Klebsiella_variicola, Raoultella_planticola, Erwinia_persicina, Enterobacter_cloacae_complex,
Klebsiella_quasipneumoniae, Klebsiella_oxytoca, Enterobacter_bugandensis, Klebsiella_michiganensis,
Serratia_marcescens, Citrobacter_amalonaticus, Citrobacter_portucalensis, Kluyvera_ascorbata,
Citrobacter_braakii, Leclercia_adecarboxylata
PWY-7184: pyrimidine deoxyribonucleotides de novo biosynthesis I
Escherichia_coli, Klebsiella_pneumoniae, Haemophilus_parainfluenzae, Klebsiella_variicola,
Citrobacter_freundii, Enterobacter_bugandensis, Enterobacter_cloacae_complex, Serratia_marcescens,
Citrobacter_portucalensis, Kluyvera_ascorbata, Pseudomonas_fragi, Pseudomonas_lundensis,
Citrobacter_braakii
PWY-7204: pyridoxal 5′-phosphate salvage II (plants)
Escherichia_coli, Klebsiella_pneumoniae, Enterobacter_cloacae_complex, Citrobacter_freundii,
Citrobacter_youngae, Citrobacter_braakii
PWY-7210: pyrimidine deoxyribonucleotides biosynthesis from CTP
Escherichia_coli, Klebsiella_pneumoniae, Haemophilus_parainfluenzae, Enterobacter_bugandensis,
Enterobacter_cloacae_complex, Serratia_marcescens, Kluyvera_ascorbata, Pseudomonas_fragi,
Pseudomonas_lundensis
PWY-7211: superpathway of pyrimidine deoxyribonucleotides de novo biosynthesis
Escherichia_coli, Klebsiella_pneumoniae, Serratia_marcescens, Enterobacter_cloacae_complex
PWY-7242: D-fructuronate degradation
Escherichia_coli, Klebsiella_pneumoniae, Faecalibacterium_prausnitzii, Hungatella_hathewayi,
Citrobacter_freundii, Bacteroides_fragilis, Clostridium_aldenense, Clostridium_sp_CAG_58,
Intestinimonas_butyriciproducens, Clostridiales_bacterium_1_7_47FAA, Klebsiella_oxytoca,
Citrobacter_youngae, Lactobacillus_rhamnosus, Citrobacter_amalonaticus, Citrobacter_portucalensis,
Kluyvera_ascorbata, Pseudoflavonifractor_capillosus, Citrobacter_braakii, Enterobacter_cloacae_complex
PWY-7269: mitochondrial DPH production (yeast)
Escherichia_coli, Klebsiella_pneumoniae, Klebsiella_variicola, Kluyvera_cryocrescens,
Pseudomonas_aeruginosa_group, Raoultella_ornithinolytica, Raoultella_planticola,
Enterobacter_cloacae_complex, Klebsiella_quasipneumoniae, Kluyvera_ascorbata, Leclercia_adecarboxylata
PWY-7282: 4-amino-2-methyl-5-diphosphomethylpyrimidine biosynthesis II
Escherichia_coli, Hafnia_alvei, Bacteroides_uniformis, Bacteroides_ovatus, Klebsiella_pneumoniae,
Klebsiella_variicola, Pseudomonas_aeruginosa_group, Bacteroides_fragilis, Enterobacter_cloacae_complex,
Bacteroides_xylanisolvens, Haemophilus_parainfluenzae, Haemophilus_sp_HMSC71H05,
Streptococcus_salivarius, Streptococcus_vestibularis, Catenibacterium_mitsuokai, Citrobacter_freundii,
Serratia_marcescens, Bacteroides_sp_D2, Citrobacter_portucalensis, Kluyvera_ascorbata,
Streptococcus_pasteurianus, Pseudomonas_lundensis, Bacteroides_sp_OM05_12, Citrobacter_braakii
PWY-7392: taxadiene biosynthesis (engineered)
Klebsiella_pneumoniae
PWY-7446: sulfoquinovose degradation I
Escherichia_coli, Klebsiella_pneumoniae, Enterobacter_cloacae_complex, Citrobacter_freundii,
Citrobacter_youngae, Enterobacter_bugandensis, Citrobacter_braakii, Klebsiella_oxytoca,
Leclercia_adecarboxylata
PWY-7456: &beta;-(1,4)-mannan degradation
Eubacterium_siraeum, Roseburia_faecis, Coprococcus_eutactus
PWY0-1298: superpathway of pyrimidine deoxyribonucleosides degradation
Escherichia_coli, Klebsiella_pneumoniae, Hungatella_hathewayi, Klebsiella_variicola, Raoultella_planticola,
Clostridium_aldenense, Clostridium_clostridioforme, Enterobacter_cloacae_complex, Streptococcus_infantis,
Streptococcus_mitis, Streptococcus_oralis, Streptococcus_parasanguinis, Citrobacter_freundii,
Citrobacter_youngae, Clostridiales_bacterium_1_7_47FAA, Enterobacter_bugandensis, Serratia_marcescens,
Citrobacter_amalonaticus, Citrobacter_portucalensis, Kluyvera_ascorbata, Citrobacter_braakii,
Leclercia_adecarboxylata
PWY3O-355: stearate biosynthesis III (fungi)
Pseudomonas_aeruginosa_group, Bacteroides_vulgatus, Bacteroides_uniformis, Bacteroides_dorei,
Bifidobacterium_longum, Bacteroides_sartorii
PWY4FS-7: phosphatidylglycerol biosynthesis I (plastidic)
Escherichia_coli, Hafnia_alvei, Klebsiella_pneumoniae, Fusicatenibacter_saccharivorans, Roseburia_intestinalis,
Klebsiella_variicola, Pseudomonas_aeruginosa_group, Raoultella_planticola, Enterobacter_cloacae_complex,
Ruminococcus_gnavus, Haemophilus_parainfluenzae, Haemophilus_sp_HMSC71H05, Streptococcus_salivarius,
Streptococcus_vestibularis, Clostridium_leptum, Citrobacter_freundii, Streptococcus_thermophilus,
Klebsiella_michiganensis, Serratia_marcescens, Citrobacter_youngae, Faecalibacterium_prausnitzii,
Citrobacter_amalonaticus, Citrobacter_portucalensis, Kluyvera_ascorbata, Catenibacterium_mitsuokai,
Fusobacterium_mortiferum, Citrobacter_braakii, Klebsiella_oxytoca
PWY4FS-8: phosphatidylglycerol biosynthesis II (non-plastidic)
Escherichia_coli, Hafnia_alvei, Klebsiella_pneumoniae, Fusicatenibacter_saccharivorans, Roseburia_intestinalis,
Klebsiella_variicola, Pseudomonas_aeruginosa_group, Raoultella_planticola, Enterobacter_cloacae_complex,
Ruminococcus_gnavus, Haemophilus_parainfluenzae, Haemophilus_sp_HMSC71H05, Streptococcus_salivarius,
Streptococcus_vestibularis, Clostridium_leptum, Citrobacter_freundii, Streptococcus_thermophilus,
Klebsiella_michiganensis, Serratia_marcescens, Citrobacter_youngae, Faecalibacterium_prausnitzii,
Citrobacter_amalonaticus, Citrobacter_portucalensis, Kluyvera_ascorbata, Catenibacterium_mitsuokai,
Fusobacterium_mortiferum, Citrobacter_braakii, Klebsiella_oxytoca
PWY4LZ-257: superpathway of fermentation (Chlamydomonas reinhardtii)
Escherichia_coli, Klebsiella_pneumoniae, Raoultella_ornithinolytica, Citrobacter_freundii,
Enterobacter_cloacae_complex
PWY66-389: phytol degradation
Escherichia_coli, Hafnia_alvei, Pseudomonas_aeruginosa_group, Raoultella_ornithinolytica,
Raoultella_planticola, Clostridium_citroniae, Klebsiella_pneumoniae, Klebsiella_quasipneumoniae,
Hafnia_paralvei, Enterobacter_cloacae_complex, Kluyvera_ascorbata, Haemophilus_parainfluenzae,
Klebsiella_oxytoca, Leclercia_adecarboxylata
PYRIDNUCSAL-PWY: D salvage pathway I (PNC VI cycle)
Escherichia_coli, Klebsiella_pneumoniae, Pseudomonas_aeruginosa_group, Serratia_marcescens,
Klebsiella_oxytoca
RIBOSYN2-PWY: flavin biosynthesis I (bacteria and plants)
Acidaminococcus_intestini, Akkermansia_muciniphila, Bacteroides_vulgatus, Ruminococcus_torques,
Dialister_succinatiphilus, Escherichia_coli, Klebsiella_pneumoniae, Eubacterium_rectale,
Megamonas_funiformis, Megamonas_hypermegale, Parabacteroides_distasonis, Bacteroides_caccae,
Bacteroides_faecis, Bacteroides_finegoldii, Bacteroides_fluxus, Bacteroides_massiliensis, Bacteroides_stercoris,
Bacteroides_uniformis, Blautia_obeum, Eubacterium_hallii, Lachnospira_pectinoschiza,
Parabacteroides_merdae, Phascolarctobacterium_faecium, Roseburia_faecis, Bacteroides_ovatus,
Bacteroides_salyersiae, Bilophila_wadsworthia, Blautia_wexlerae, Coprococcus_comes, Eubacterium_eligens,
Faecalibacterium_prausnitzii, Odoribacter_splanchnicus, Bacteroides_cellulosilyticus,
Bacteroides_thetaiotaomicron, Clostridium_bolteae_CAG_59, Hungatella_hathewayi, Clostridium_aldenense,
Clostridium_bolteae, Roseburia_intestinalis, Anaerostipes_hadrus, Citrobacter_braakii, Klebsiella_variicola,
Pseudomonas_aeruginosa_group, Agathobaculum_butyriciproducens, Bacteroides_fragilis,
Bacteroides_xylanisolvens, Dorea_longicatena, Paraprevotella_xylaniphila, Roseburia_hominis,
Flavonifractor_plautii, Clostridium_clostridioforme, Anaerotignum_lactatifermentans,
Clostridium_sp_CAG_299, Bacteroides_galacturonicus, Bacteroides_intestinalis,
Enterobacter_cloacae_complex, Lactobacillus_rogosae, Roseburia_sp_CAG_303, Bacteroides_dorei,
Eubacterium_sp_CAG_274, Bacteroides_coprocola, Dorea_formicigenerans, Holdemanella_biformis,
Barnesiella_intestinihominis, Clostridium_sp_CAG_167, Haemophilus_parainfluenzae,
Haemophilus_sp_HMSC71H05, Ruminococcus_callidus, Bacteroides_eggerthii, Catenibacterium_mitsuokai,
Eubacterium_ramulus, Phascolarctobacterium_succinatutens, Eubacterium_sp_CAG_38, Clostridium_innocuum,
Paraprevotella_clara, Clostridium_spiroforme, Clostridium_symbiosum, Veillonella_dispar, Veillonella_parvula,
Veillonella_rogosae, Adlercreutzia_equolifaciens, Asaccharobacter_celatus, Klebsiella_oxytoca,
Parabacteroides_johnsonii, Bacteroides_coprophilus, Dialister_sp_CAG_357, Citrobacter_youngae,
Prevotella_copri, Prevotella_sp_AM42_24, Pseudomonas_fragi, Coprobacter_fastidiosus, Bacteroides_clarus,
Clostridiales_bacterium_1_7_47FAA, Enterobacter_bugandensis, Serratia_marcescens, Coprococcus_eutactus,
Coprococcus_catus, Anaerostipes_caccae, Pseudomonas_helleri, Bacteroides_sp_D2, Citrobacter_amalonaticus,
Streptococcus_lutetiensis, Veillonella_infantium, Citrobacter_portucalensis, Kluyvera_ascorbata,
Slackia_isoflavoniconvertens, Streptococcus_pasteurianus, Veillonella_atypica, Veillonella_tobetsuensis,
Oxalobacter_formigenes, Bifidobacterium_longum, Bacteroides_sartorii, Fusobacterium_mortiferum,
Pseudoflavonifractor_sp_An184, Acidaminococcus_fermentans, Prevotella_sp_CAG_279,
Prevotella_sp_CAG_1031, Ruminococcus_sp_CAG_403, Parabacteroides_sp_CAG_409,
Flavonifractor_sp_An82, Clostridium_sp_CAG_253, Megasphaera_elsdenii, Parabacteroides_goldsteinii,
Pseudomonas_lundensis, Pseudomonas_psychrophila, Pseudomonas_taetrolens, Bacteroides_sp_OM05_12,
Intestinibacter_bartlettii, Bacteroides_sp_OM08_11, Coprobacillus_cateniformis, Bacteroides_nordii,
Leclercia_adecarboxylata, Ruminococcaceae_bacterium_D16
SO4ASSIM-PWY: assimilatory sulfate reduction I
Escherichia_coli, Citrobacter_freundii, Citrobacter_braakii, Klebsiella_pneumoniae, Klebsiella_variicola,
Pseudomonas_aeruginosa_group, Raoultella_ornithinolytica, Raoultella_planticola,
Enterobacter_cloacae_complex, Haemophilus_parainfluenzae, Klebsiella_oxytoca, Klebsiella_quasipneumoniae,
Hafnia_paralvei, Enterobacter_bugandensis, Klebsiella_michiganensis, Serratia_marcescens,
Citrobacter_youngae, Odoribacter_laneus, Citrobacter_amalonaticus, Citrobacter_portucalensis,
Kluyvera_ascorbata, Pseudomonas_lundensis, Leclercia_adecarboxylata
SULFATE-CYS-PWY: superpathway of sulfate assimilation and cysteine biosynthesis
Escherichia_coli, Citrobacter_braakii, Klebsiella_pneumoniae, Raoultella_ornithinolytica, Raoultella_planticola,
Enterobacter_cloacae_complex, Haemophilus_parainfluenzae, Klebsiella_variicola, Citrobacter_freundii,
Enterobacter_bugandensis, Klebsiella_michiganensis, Serratia_marcescens, Citrobacter_youngae,
Citrobacter_amalonaticus, Citrobacter_portucalensis, Pseudomonas_lundensis
TCA: TCA cycle I (prokaryotic)
Escherichia_coli, Klebsiella_pneumoniae, Klebsiella_variicola, Raoultella_ornithinolytica,
Enterobacter_cloacae_complex, Citrobacter_freundii, Citrobacter_youngae, Klebsiella_quasipneumoniae,
Hafnia_paralvei, Enterobacter_bugandensis, Klebsiella_michiganensis, Serratia_marcescens,
Citrobacter_portucalensis, Kluyvera_ascorbata, Citrobacter_braakii, Klebsiella_oxytoca
TEICHOICACID-PWY: poly(glycerol phosphate) wall teichoic acid biosynthesis
Parabacteroides_distasonis, Bacteroides_thetaiotaomicron, Bacteroides_vulgatus, Ruminococcus_torques,
Lactobacillus_plantarum
THISYRA-PWY: superpathway of thiamine diphosphate biosynthesis III (eukaryotes)
Ruminococcus_torques, Dorea_longicatena, Eubacterium_rectale, Bacteroides_uniformis,
Faecalibacterium_prausnitzii, Roseburia_faecis, Bacteroides_ovatus, Ruminococcus_gnavus,
Roseburia_intestinalis, Bacteroides_fragilis, Clostridium_aldenense, Clostridium_scindens, Eubacterium_hallii,
Bacteroides_xylanisolvens, Holdemanella_biformis, Roseburia_hominis, Catenibacterium_mitsuokai,
Phascolarctobacterium_succinatutens, Clostridium_innocuum, Clostridiales_bacterium_1_7_47FAA,
Clostridium_saccharolyticum, Bifidobacterium_bifidum, Eubacterium_callanderi, Fusobacterium_mortiferum,
Bacteroides_sp_OM08_11
Pathway - IBS-C (Pathway: microbe)
ARGININE-SYN4-PWY: L-ornithine biosynthesis II
Bacteroides_caccae, Bacteroides_cellulosilyticus, Bacteroides_dorei, Bacteroides_massiliensis,
Bacteroides_ovatus, Bacteroides_stercoris, Bacteroides_uniformis, Bacteroides_thetaiotaomicron,
Bacteroides_vulgatus, Bacteroides_xylanisolvens, Bacteroides_fragilis, Bacteroides_faecis,
Bacteroides_finegoldii, Bacteroides_intestinalis, Paraprevotella_xylaniphila, Paraprevotella_clara,
Bacteroides_coprocola, Bacteroides_coprophilus, Bacteroides_plebeius, Bacteroides_stercorirosoris,
Bacteroides_eggerthii, Catenibacterium_mitsuokai, Bacteroides_clarus, Bacteroides_salyersiae,
Bacteroides_sartorii, Bacteroides_faecis_CAG_32, Bacteroides_nordii, Bacteroides_sp_OM08_11,
Citrobacter_freundii, Bacteroides_sp_CAG_443, Klebsiella_pneumoniae
ECASYN-PWY: enterobacterial common antigen biosynthesis
Citrobacter_amalonaticus, Enterobacter_cloacae_complex, Hafnia_alvei, Escherichia_coli, Morganella_morganii,
Klebsiella_oxytoca, Klebsiella_aerogenes, Citrobacter_werkmanii, Citrobacter_braakii, Hafnia_paralvei,
Citrobacter_freundii, Klebsiella_quasipneumoniae
GLUCARDEG-PWY: D-glucarate degradation I
Escherichia_coli, Citrobacter_amalonaticus, Enterobacter_cloacae_complex, Klebsiella_pneumoniae,
Clostridium_aldenense, Klebsiella_oxytoca, Citrobacter_freundii, Klebsiella_aerogenes, Citrobacter_youngae,
Clostridium_citroniae, Clostridium_clostridioforme, Klebsiella_quasipneumoniae
GLUCUROCAT-PWY: superpathway of &beta;-D-glucuronosides degradation
Escherichia_coli, Faecalibacterium_prausnitzii, Bacteroides_fragilis, Intestinimonas_butyriciproducens,
Lactobacillus_rhamnosus, Klebsiella_oxytoca, Hungatella_hathewayi, Citrobacter_braakii,
Citrobacter_werkmanii, Citrobacter_freundii, Klebsiella_pneumoniae
GLYCOLYSIS: glycolysis I (from glucose 6-phosphate)
Escherichia_coli, Bacteroides_fragilis, Citrobacter_amalonaticus, Enterobacter_cloacae_complex,
Haemophilus_parainfluenzae, Hafnia_alvei, Hafnia_paralvei, Bacteroides_cellulosilyticus,
Parabacteroides_distasonis, Morganella_morganii, Klebsiella_oxytoca, Bacteroides_salyersiae,
Klebsiella_aerogenes, Catenibacterium_mitsuokai, Citrobacter_freundii, Streptococcus_infantis,
Citrobacter_braakii, Citrobacter_werkmanii, Haemophilus_sp_HMSC71H05, Bacteroides_nordii,
Klebsiella_pneumoniae, Bacteroides_xylanisolvens, Cronobacter_sakazakii, Klebsiella_quasipneumoniae,
Klebsiella_variicola
GOLPDLCAT-PWY: superpathway of glycerol degradation to 1,3-propanediol
Anaerostipes_hadrus, Ruminococcus_torques, Flavonifractor_plautii, Citrobacter_amalonaticus,
Citrobacter_europaeus, Enterobacter_cloacae_complex, Hafnia_paralvei, Klebsiella_oxytoca,
Eubacterium_hallii, Citrobacter_braakii, Citrobacter_werkmanii, Klebsiella_grimontii, Klebsiella_pneumoniae,
Citrobacter_freundii, Citrobacter_youngae, Escherichia_coli
HISTSYN-PWY: L-histidine biosynthesis
Alistipes_shahii, Bacteroides_caccae, Bacteroides_cellulosilyticus, Bacteroides_dorei, Bacteroides_massiliensis,
Bacteroides_ovatus, Bacteroides_salyersiae, Bacteroides_stercoris, Bacteroides_uniformis,
Ruminococcus_torques, Eubacterium_sp_CAG_251, Hungatella_hathewayi, Clostridium_bolteae,
Parabacteroides_merdae, Ruminococcaceae_bacterium_D16, Alistipes_finegoldii, Bacteroides_vulgatus,
Bacteroides_xylanisolvens, Barnesiella_intestinihominis, Bifidobacterium_adolescentis, Collinsella_aerofaciens,
Dorea_formicigenerans, Eubacterium_sp_CAG_38, Faecalibacterium_prausnitzii, Odoribacter_splanchnicus,
Eubacterium_siraeum, Ruminococcus_bromii, Ruminococcus_champanellensis, Bacteroides_fragilis,
Blautia_obeum, Citrobacter_amalonaticus, Enterobacter_cloacae_complex, Erysipelatoclostridium_ramosum,
Eubacterium_ventriosum, Hafnia_alvei, Parabacteroides_distasonis, Roseburia_inulinivorans,
Veillonella_atypica, Veillonella_parvula, Alistipes_sp_An66, Eisenbergiella_tayi, Escherichia_coli,
Flavonifractor_sp_An10, Klebsiella_pneumoniae, Methanobrevibacter_smithii,
Agathobaculum_butyriciproducens, Bacteroides_finegoldii, Bacteroides_thetaiotaomicron,
Bifidobacterium_pseudocatenulatum, Clostridium_bolteae_CAG_59, Dorea_longicatena, Flavonifractor_plautii,
Clostridium_aldenense, Bifidobacterium_longum, Eisenbergiella_massiliensis, Firmicutes_bacterium_CAG_94,
Parasutterella_excrementihominis, Roseburia_intestinalis, Ruthenibacterium_lactatiformans,
Alistipes_onderdonkii, Bacteroides_intestinalis, Coprobacter_fastidiosus, Pseudomonas_aeruginosa_group,
Alistipes_indistinctus, Paraprevotella_xylaniphila, Odoribacter_laneus, Bacteroides_sp_CAG_144,
Paraprevotella_clara, Prevotella_sp_CAG_5226, Anaerotignum_lactatifermentans,
Lawsonibacter_asaccharolyticus, Eubacterium_sp_CAG_180, Proteobacteria_bacterium_CAG_139,
Bacteroides_coprocola, Bacteroides_coprophilus, Bacteroides_plebeius, Dialister_sp_CAG_357,
Parabacteroides_johnsonii, Prevotella_sp_CAG_279, Clostridium_clostridioforme, Bacteroides_stercorirosoris,
Morganella_morganii, Bacteroides_eggerthii, Bacteroides_faecichinchillae, Parabacteroides_goldsteinii,
Parabacteroides_gordonii, Eggerthella_lenta, Streptococcus_salivarius, Parabacteroides_sp_CAG_409,
Klebsiella_oxytoca, Butyricimonas_synergistica, Coprococcus_catus, Cloacibacillus_evryensis,
Intestinimonas_butyriciproducens, Pseudoflavonifractor_sp_An184, Bacteroides_faecis,
Anaerotruncus_sp_CAG_528, Clostridium_innocuum, Clostridium_spiroforme,
Firmicutes_bacterium_CAG_424, Clostridium_scindens, Catenibacterium_mitsuokai, Roseburia_hominis,
Clostridium_leptum, Bacteroides_clarus, Coprobacter_secundus, Megamonas_funiformis, Bacteroides_sartorii,
Prevotella_copri, Faecalitalea_cylindroides, Klebsiella_aerogenes, Bacteroides_faecis_CAG_32,
Ruminococcus_sp_CAG_488, Bifidobacterium_bifidum, Butyricimonas_virosa, Eubacterium_sp_CAG_274,
Candidatus_Stoquefichus_sp_KLE1796, Blautia_producta, Gordonibacter_pamelaeae, Lactobacillus_ruminis,
Citrobacter_braakii, Citrobacter_werkmanii, Clostridium_sp_CAG_678, Acinetobacter_ursingii,
Streptococcus_thermophilus, Acidaminococcus_fermentans, Megasphaera_elsdenii, Bacteroides_nordii,
Anaerotruncus_colihominis, Collinsella_stercoris, Megasphaera_sp_MJR8396C, Blautia_coccoides,
Haemophilus_parainfluenzae, Absiella_dolichum, Bacteroides_sp_OM08_11, Slackia_isoflavoniconvertens,
Clostridiales_bacterium_1_7_47FAA, Coprobacillus_cateniformis, Clostridium_asparagiforme,
Dysgonomonas_sp_37_18, Megasphaera_sp_DISK_18, Bifidobacterium_breve, Citrobacter_freundii,
Citrobacter_youngae, Streptococcus_pasteurianus, Prevotella_sp_AM42_24, Prevotella_sp_CAG_1092,
Bacteroides_sp_CAG_443, Coprobacter_sp, Bifidobacterium_kashiwanohense, Megasphaera_stantonii,
Klebsiella_quasipneumoniae, Klebsiella_variicola, Lactobacillus_plantarum, Clostridium_lavalense
KETOGLUCONMET-PWY: ketogluconate metabolism
Escherichia_coli, Citrobacter_amalonaticus, Enterobacter_cloacae_complex, Hafnia_alvei, Hafnia_paralvei,
Klebsiella_pneumoniae, Escherichia_marmotae, Pseudomonas_aeruginosa_group, Enterococcus_faecium,
Klebsiella_oxytoca, Citrobacter_koseri, Citrobacter_braakii, Citrobacter_freundii, Cronobacter_sakazakii,
Lactobacillus_plantarum
METH-ACETATE-PWY: methanogenesis from acetate
Blautia_wexlerae, Blautia_obeum, Fusicatenibacter_saccharivorans, Ruminococcus_torques,
Blautia_sp_CAG_257, Blautia_hydrogenotrophica
GLIPASYN-PWY: lipid IVA biosynthesis (E. coli)
Bacteroides_stercoris, Bacteroides_uniformis, Escherichia_coli, Citrobacter_amalonaticus,
Enterobacter_cloacae_complex, Haemophilus_parainfluenzae, Hafnia_alvei, Hafnia_paralvei,
Escherichia_marmotae, Parasutterella_excrementihominis, Bacteroides_cellulosilyticus, Bacteroides_intestinalis,
Pseudomonas_aeruginosa_group, Proteobacteria_bacterium_CAG_139, Morganella_morganii,
Bacteroides_fragilis, Klebsiella_oxytoca, Klebsiella_aerogenes, Catenibacterium_mitsuokai, Citrobacter_braakii,
Citrobacter_youngae, Acinetobacter_ursingii, Haemophilus_sp_HMSC71H05, Klebsiella_pneumoniae,
Turicimonas_muris, Escherichia_albertii, Citrobacter_freundii, Klebsiella_quasipneumoniae
P124-PWY: Bifidobacterium shunt
Collinsella_aerofaciens, Bifidobacterium_pseudocatenulatum, Bifidobacterium_longum,
Pseudomonas_aeruginosa_group, Bifidobacterium_dentium
P161-PWY: acetylene degradation (anaerobic)
Flavonifractor_plautii, Citrobacter_amalonaticus, Citrobacter_europaeus, Enterobacter_cloacae_complex,
Enterobacter_mori, Hafnia_alvei, Hafnia_paralvei, Escherichia_coli, Klebsiella_pneumoniae, Dorea_longicatena,
Clostridium_aldenense, Klebsiella_variicola, Morganella_morganii, Klebsiella_oxytoca, Klebsiella_aerogenes,
Citrobacter_freundii, Citrobacter_youngae, Streptococcus_australis, Streptococcus_parasanguinis,
Citrobacter_braakii, Citrobacter_werkmanii, Raoultella_ornithinolytica, Clostridiales_bacterium_1_7_47FAA,
Hungatella_hathewayi, Pseudoflavonifractor_capillosus, Cronobacter_sakazakii, Klebsiella_michiganensis,
Klebsiella_quasipneumoniae, Lactobacillus_plantarum
P221-PWY: octane oxidation
Escherichia_coli, Pseudomonas_aeruginosa_group, Klebsiella_pneumoniae, Klebsiella_quasipneumoniae
PENTOSE-P-PWY: pentose phosphate pathway
Bacteroides_thetaiotaomicron, Escherichia_coli, Parabacteroides_distasonis, Bacteroides_fragilis,
Citrobacter_amalonaticus, Enterobacter_cloacae_complex, Haemophilus_parainfluenzae, Hafnia_alvei,
Hafnia_paralvei, Dorea_longicatena, Clostridium_aldenense, Bacteroides_cellulosilyticus,
Bacteroides_xylanisolvens, Morganella_morganii, Klebsiella_oxytoca, Hungatella_hathewayi,
Clostridium_clostridioforme, Bifidobacterium_bifidum, Blautia_coccoides, Citrobacter_braakii,
Citrobacter_werkmanii, Haemophilus_sp_HMSC71H05, Klebsiella_pneumoniae, Eisenbergiella_tayi,
Clostridiales_bacterium_1_7_47FAA, Citrobacter_freundii, Cronobacter_sakazakii,
Klebsiella_quasipneumoniae, Lactobacillus_plantarum
PHOSLIPSYN-PWY: superpathway of phospholipid biosynthesis I (bacteria)
Escherichia_coli, Fusicatenibacter_saccharivorans, Ruminococcus_gnavus, Citrobacter_amalonaticus,
Enterobacter_cloacae_complex, Haemophilus_parainfluenzae, Hafnia_alvei, Hafnia_paralvei,
Escherichia_marmotae, Pseudomonas_aeruginosa_group, Morganella_morganii, Klebsiella_oxytoca,
Clostridium_leptum, Klebsiella_aerogenes, Catenibacterium_mitsuokai, Citrobacter_braakii,
Haemophilus_sp_HMSC71H05, Citrobacter_freundii, Klebsiella_quasipneumoniae, Klebsiella_variicola
POLYISOPRENSYN-PWY: polyisoprenoid biosynthesis (E. coli)
Escherichia_coli, Citrobacter_amalonaticus, Hafnia_paralvei, Klebsiella_pneumoniae, Morganella_morganii,
Klebsiella_oxytoca, Klebsiella_aerogenes, Citrobacter_braakii, Citrobacter_freundii, Cronobacter_sakazakii,
Klebsiella_quasipneumoniae, Klebsiella_variicola
PWY-4321: L-glutamate degradation IV
Hafnia_paralvei, Escherichia_coli, Klebsiella_pneumoniae
PWY-5030: L-histidine degradation III
Alistipes_putredinis, Bacteroides_dorei, Bacteroides_massiliensis, Bacteroides_ovatus, Bacteroides_stercoris,
Bacteroides_thetaiotaomicron, Bacteroides_uniformis, Bacteroides_vulgatus, Bacteroides_xylanisolvens,
Alistipes_shahii, Bacteroides_fragilis, Streptococcus_parasanguinis, Alistipes_onderdonkii, Alistipes_finegoldii,
Bacteroides_eggerthii, Bacteroides_clarus, Bacteroides_faecis, Bacteroides_sartorii, Catenibacterium_mitsuokai,
Streptococcus_australis, Acidaminococcus_fermentans, Alistipes_timonensis, Bacteroides_sp_OM08_11,
Streptococcus_sp_F0442
PWY-5083: D(P)/DPH interconversion
Escherichia_coli, Citrobacter_amalonaticus, Citrobacter_europaeus, Enterobacter_cloacae_complex,
Escherichia_marmotae, Klebsiella_oxytoca, Klebsiella_aerogenes, Citrobacter_braakii, Citrobacter_werkmanii,
Raoultella_ornithinolytica, Escherichia_albertii, Klebsiella_pneumoniae, Citrobacter_youngae,
Citrobacter_freundii, Klebsiella_quasipneumoniae, Klebsiella_variicola
PWY-5104: L-isoleucine biosynthesis IV
Escherichia_coli, Parabacteroides_distasonis, Parabacteroides_merdae, Citrobacter_amalonaticus,
Citrobacter_europaeus, Eggerthella_lenta, Enterobacter_cloacae_complex, Dorea_longicatena,
Pseudomonas_aeruginosa_group, Parabacteroides_goldsteinii, Citrobacter_braakii, Citrobacter_werkmanii,
Candidatus_Methanomassiliicoccus_intestinalis, Citrobacter_freundii, Citrobacter_youngae,
Klebsiella_pneumoniae, Klebsiella_quasipneumoniae
PWY-5189: tetrapyrrole biosynthesis II (from glycine)
Escherichia_coli, Clostridium_perfringens, Enterobacter_cloacae_complex, Escherichia_marmotae,
Pseudomonas_aeruginosa_group, Klebsiella_oxytoca, Intestinimonas_butyriciproducens, Lactobacillus_reuteri,
Klebsiella_aerogenes, Streptococcus_australis, Streptococcus_parasanguinis,
Candidatus_Methanomassiliicoccus_intestinalis, Clostridium_asparagiforme, Klebsiella_pneumoniae,
Citrobacter_amalonaticus
PWY-5345: superpathway of L-methionine biosynthesis (by sulfhydrylation)
PWY-5484: glycolysis II (from fructose 6-phosphate)
Escherichia_coli, Bacteroides_fragilis, Citrobacter_amalonaticus, Enterobacter_cloacae_complex,
Haemophilus_parainfluenzae, Hafnia_alvei, Hafnia_paralvei, Bacteroides_cellulosilyticus,
Parabacteroides_distasonis, Morganella_morganii, Klebsiella_oxytoca, Bacteroides_salyersiae,
Klebsiella_aerogenes, Catenibacterium_mitsuokai, Citrobacter_braakii, Citrobacter_werkmanii,
Citrobacter_youngae, Haemophilus_sp_HMSC71H05, Bacteroides_nordii, Klebsiella_pneumoniae,
Bacteroides_xylanisolvens, Citrobacter_freundii, Cronobacter_sakazakii, Klebsiella_quasipneumoniae,
Klebsiella_variicola
PWY-5675: nitrate reduction V (assimilatory)
Citrobacter_amalonaticus, Enterobacter_cloacae_complex, Escherichia_coli, Klebsiella_variicola,
Klebsiella_oxytoca, Klebsiella_aerogenes, Citrobacter_braakii, Raoultella_ornithinolytica,
Klebsiella_pneumoniae, Citrobacter_freundii, Klebsiella_quasipneumoniae
PWY-5695: inosine 5′-phosphate degradation
Alistipes_putredinis, Alistipes_shahii, Anaerostipes_hadrus, Bacteroides_caccae, Bacteroides_cellulosilyticus,
Bacteroides_dorei, Bacteroides_massiliensis, Bacteroides_ovatus, Bacteroides_salyersiae, Bacteroides_stercoris,
Bacteroides_uniformis, Blautia_wexlerae, Ruminococcus_torques, Clostridiales_bacterium_1_7_47FAA,
Dorea_longicatena, Escherichia_coli, Faecalibacterium_prausnitzii, Hungatella_hathewayi,
Clostridium_aldenense, Clostridium_bolteae, Ruminococcus_bicirculans, Alistipes_finegoldii, Alistipes_inops,
Bacteroides_vulgatus, Bacteroides_xylanisolvens, Barnesiella_intestinihominis, Blautia_obeum,
Dialister_invisus, Eubacterium_eligens, Eubacterium_ramulus, Eubacterium_ventriosum,
Fusicatenibacter_saccharivorans, Odoribacter_splanchnicus, Roseburia_faecis,
Agathobaculum_butyriciproducens, Bacteroides_fragilis, Citrobacter_amalonaticus, Clostridium_perfringens,
Coprococcus_catus, Coprococcus_comes, Enterobacter_cloacae_complex, Enterococcus_faecalis,
Haemophilus_parainfluenzae, Hafnia_alvei, Hafnia_paralvei, Clostridium_symbiosum,
Parabacteroides_distasonis, Roseburia_inulinivorans, Veillonella_atypica, Veillonella_parvula,
Eisenbergiella_tayi, Klebsiella_pneumoniae, Bacteroides_finegoldii, Bacteroides_thetaiotaomicron,
Clostridium_scindens, Eisenbergiella_massiliensis, Escherichia_marmotae, Firmicutes_bacterium_CAG_94,
Flavonifractor_plautii, Clostridium_clostridioforme, Pseudomonas_aeruginosa_group, Roseburia_intestinalis,
Alistipes_onderdonkii, Bacteroides_intestinalis, Coprococcus_eutactus, Intestinimonas_butyriciproducens,
Parabacteroides_goldsteinii, Lachnospira_pectinoschiza, Lactobacillus_rogosae, Bacteroides_galacturonicus,
Bacteroides_coprocola, Bacteroides_coprophilus, Bacteroides_plebeius, Coprobacter_fastidiosus,
Prevotella_sp_CAG_279, Morganella_morganii, Bacteroides_eggerthii, Bacteroides_faecichinchillae,
Parabacteroides_gordonii, Streptococcus_salivarius, Klebsiella_oxytoca, Clostridium_citroniae,
Bilophila_wadsworthia, Bacteroides_faecis, Desulfovibrio_piger, Parabacteroides_johnsonii,
Citrobacter_freundii, Catenibacterium_mitsuokai, Bacteroides_clarus, Coprobacter_secundus,
Klebsiella_aerogenes, Alistipes_indistinctus, Bacteroides_sartorii, Faecalitalea_cylindroides, Blautia_coccoides,
Clostridium_asparagiforme, Streptococcus_australis, Streptococcus_infantis, Citrobacter_werkmanii,
Acinetobacter_ursingii, Anaerotruncus_colihominis, Citrobacter_braakii, Raoultella_ornithinolytica,
Bacteroides_faecis_CAG_32, Blautia_hydrogenotrophica, Haemophilus_sp_HMSC71H05,
Megasphaera_elsdenii, Alistipes_timonensis, Bacteroides_nordii, Collinsella_stercoris,
Bacteroides_stercorirosoris, Coprobacter_sp, Megasphaera_sp_MJR8396C, Christensenella_minuta,
Desulfovibrio_fairfieldensis, Veillonella_sp_CAG_933, Escherichia_albertii, Ruminococcus_champanellensis,
Turicibacter_sanguinis, Bacteroides_sp_OM08_11, Intestinibacter_bartlettii, Dysgonomonas_sp_37_18,
Megasphaera_sp_DISK_18, Prevotella_copri, Prevotella_sp_CAG_891, Clostridium_saccharolyticum,
Prevotella_sp_AM42_24, Bacteroides_sp_CAG_443, Streptococcus_sp_F0442, Megasphaera_stantonii,
Streptococcus_vestibularis, Klebsiella_quasipneumoniae
PWY-5723: Rubisco shunt
Escherichia_coli, Citrobacter_amalonaticus, Enterobacter_cloacae_complex, Hafnia_paralvei,
Klebsiella_pneumoniae, Klebsiella_oxytoca, Klebsiella_aerogenes, Citrobacter_werkmanii, Citrobacter_braakii,
Citrobacter_freundii, Klebsiella_quasipneumoniae
PWY-5918: superpathway of heme b biosynthesis from glutamate
Escherichia_coli, Citrobacter_amalonaticus, Enterobacter_cloacae_complex, Pseudomonas_aeruginosa_group,
Klebsiella_oxytoca, Klebsiella_aerogenes, Citrobacter_braakii, Klebsiella_pneumoniae, Citrobacter_freundii
PWY-5971: palmitate biosynthesis (type II fatty acid synthase)
Escherichia_coli, Enterobacter_cloacae_complex, Citrobacter_braakii, Citrobacter_werkmanii,
Citrobacter_youngae, Citrobacter_freundii, Citrobacter_amalonaticus
PWY-622: starch biosynthesis
PWY-6284: superpathway of unsaturated fatty acids biosynthesis (E. coli)
Escherichia_coli, Citrobacter_europaeus, Enterobacter_cloacae_complex, Klebsiella_oxytoca,
Citrobacter_braakii, Citrobacter_werkmanii, Citrobacter_youngae, Citrobacter_freundii,
Citrobacter_amalonaticus
PWY-6507: 4-deoxy-L-threo-hex-4-enopyranuronate degradation
Clostridiales_bacterium_1_7_47FAA, Escherichia_coli, Faecalibacterium_prausnitzii, Citrobacter_amalonaticus,
Citrobacter_europaeus, Enterobacter_cloacae_complex, Hafnia_alvei, Hafnia_paralvei, Dorea_longicatena,
Hungatella_hathewayi, Clostridium_aldenense, Clostridium_bolteae, Clostridium_citroniae,
Clostridium_clostridioforme, Morganella_morganii, Klebsiella_oxytoca, Lactobacillus_rhamnosus,
Citrobacter_braakii, Citrobacter_werkmanii, Citrobacter_youngae, Anaerotruncus_colihominis,
Citrobacter_freundii
PWY-6545: pyrimidine deoxyribonucleotides de novo biosynthesis III
Escherichia_coli, Klebsiella_oxytoca, Candidatus_Methanomassiliicoccus_intestinalis, Klebsiella_pneumoniae,
Klebsiella_quasipneumoniae
PWY-6803: phosphatidylcholine acyl editing
Escherichia_coli, Citrobacter_amalonaticus, Citrobacter_europaeus, Enterobacter_cloacae_complex,
Hafnia_alvei, Hafnia_paralvei, Klebsiella_pneumoniae, Escherichia_marmotae, Morganella_morganii,
Klebsiella_oxytoca, Citrobacter_freundii, Citrobacter_youngae, Klebsiella_aerogenes, Citrobacter_koseri,
Citrobacter_braakii, Citrobacter_werkmanii, Escherichia_albertii, Klebsiella_quasipneumoniae,
Klebsiella_variicola
PWY-7184: pyrimidine deoxyribonucleotides de novo biosynthesis I
Enterobacter_cloacae_complex, Hafnia_alvei, Hafnia_paralvei, Escherichia_coli, Morganella_morganii,
Klebsiella_oxytoca, Klebsiella_aerogenes, Acinetobacter_ursingii, Citrobacter_braakii, Klebsiella_pneumoniae,
Haemophilus_parainfluenzae, Citrobacter_freundii, Citrobacter_amalonaticus, Klebsiella_quasipneumoniae
PWY-7204: pyridoxal 5′-phosphate salvage II (plants)
Escherichia_coli, Enterobacter_cloacae_complex, Klebsiella_oxytoca, Klebsiella_aerogenes,
Citrobacter_werkmanii, Citrobacter_braakii, Citrobacter_freundii, Citrobacter_youngae, Klebsiella_pneumoniae
PWY-7210: pyrimidine deoxyribonucleotides biosynthesis from CTP
Enterobacter_cloacae_complex, Hafnia_alvei, Hafnia_paralvei, Escherichia_coli, Morganella_morganii,
Klebsiella_oxytoca, Acinetobacter_ursingii, Candidatus_Methanomassiliicoccus_intestinalis,
Haemophilus_parainfluenzae, Klebsiella_pneumoniae, Klebsiella_quasipneumoniae
PWY-7211: superpathway of pyrimidine deoxyribonucleotides de novo biosynthesis
Escherichia_coli, Klebsiella_quasipneumoniae
PWY-7242: D-fructuronate degradation
Escherichia_coli, Faecalibacterium_prausnitzii, Bacteroides_fragilis, Citrobacter_amalonaticus,
Enterobacter_cloacae_complex, Hafnia_alvei, Hafnia_paralvei, Klebsiella_pneumoniae, Dorea_longicatena,
Clostridium_aldenense, Clostridium_sp_CAG_58, Intestinimonas_butyriciproducens, Lactobacillus_rhamnosus,
Morganella_morganii, Klebsiella_oxytoca, Pseudoflavonifractor_capillosus, Hungatella_hathewayi,
Citrobacter_braakii, Citrobacter_werkmanii, Citrobacter_youngae, Clostridiales_bacterium_1_7_47FAA,
Citrobacter_freundii, Cronobacter_sakazakii
PWY-7269: mitochondrial DPH production (yeast)
Enterobacter_cloacae_complex, Escherichia_coli, Pseudomonas_aeruginosa_group, Klebsiella_oxytoca,
Klebsiella_pneumoniae, Escherichia_albertii, Klebsiella_quasipneumoniae, Klebsiella_variicola
PWY-7282: 4-amino-2-methyl-5-diphosphomethylpyrimidine biosynthesis II
Bacteroides_ovatus, Bacteroides_uniformis, Escherichia_coli, Bacteroides_xylanisolvens, Bacteroides_fragilis,
Citrobacter_europaeus, Enterobacter_cloacae_complex, Haemophilus_parainfluenzae, Klebsiella_oxytoca,
Streptococcus_salivarius, Klebsiella_aerogenes, Pseudomonas_aeruginosa_group, Catenibacterium_mitsuokai,
Citrobacter_werkmanii, Citrobacter_braakii, Streptococcus_vestibularis, Citrobacter_freundii,
Citrobacter_amalonaticus, Klebsiella_quasipneumoniae
PWY-7392: taxadiene biosynthesis (engineered)
PWY-7446: sulfoquinovose degradation I
Escherichia_coli, Citrobacter_amalonaticus, Enterobacter_cloacae_complex, Klebsiella_pneumoniae,
Citrobacter_braakii, Citrobacter_werkmanii, Citrobacter_freundii, Citrobacter_youngae, Klebsiella_oxytoca
PWY-7456: &beta;-(1,4)-mannan degradation
Eubacterium_siraeum, Coprococcus_eutactus, Roseburia_faecis
PWY0-1298: superpathway of pyrimidine deoxyribonucleosides degradation
Escherichia_coli, Citrobacter_amalonaticus, Citrobacter_europaeus, Enterobacter_cloacae_complex,
Hafnia_alvei, Hafnia_paralvei, Klebsiella_pneumoniae, Dorea_longicatena, Morganella_morganii,
Klebsiella_oxytoca, Hungatella_hathewayi, Clostridium_aldenense, Klebsiella_aerogenes,
Streptococcus_australis, Streptococcus_parasanguinis, Clostridium_clostridioforme, Citrobacter_braakii,
Citrobacter_werkmanii, Escherichia_albertii, Citrobacter_freundii, Citrobacter_youngae, Streptococcus_mitis,
Cronobacter_sakazakii, Klebsiella_quasipneumoniae, Klebsiella_variicola
PWY30-355: stearate biosynthesis III (fungi)
Bacteroides_dorei, Bacteroides_uniformis, Bacteroides_vulgatus, Pseudomonas_aeruginosa_group,
Bifidobacterium_longum, Bacteroides_sartorii
PWY4FS-7: phosphatidylglycerol biosynthesis I (plastidic)
Escherichia_coli, Fusicatenibacter_saccharivorans, Ruminococcus_gnavus, Citrobacter_amalonaticus,
Enterobacter_cloacae_complex, Haemophilus_parainfluenzae, Hafnia_alvei, Hafnia_paralvei,
Klebsiella_pneumoniae, Escherichia_marmotae, Roseburia_intestinalis, Pseudomonas_aeruginosa_group,
Faecalibacterium_prausnitzii, Morganella_morganii, Streptococcus_salivarius, Klebsiella_oxytoca,
Streptococcus_thermophilus, Streptococcus_vestibularis, Clostridium_leptum, Klebsiella_aerogenes,
Catenibacterium_mitsuokai, Citrobacter_braakii, Haemophilus_sp_HMSC71H05, Citrobacter_freundii,
Klebsiella_quasipneumoniae, Klebsiella_variicola
PWY4FS-8: phosphatidylglycerol biosynthesis II (non-plastidic)
Escherichia_coli, Fusicatenibacter_saccharivorans, Ruminococcus_gnavus, Citrobacter_amalonaticus,
Enterobacter_cloacae_complex, Haemophilus_parainfluenzae, Hafnia_alvei, Hafnia_paralvei,
Klebsiella_pneumoniae, Escherichia_marmotae, Roseburia_intestinalis, Pseudomonas_aeruginosa_group,
Faecalibacterium_prausnitzii, Morganella_morganii, Streptococcus_salivarius, Klebsiella_oxytoca,
Streptococcus_thermophilus, Streptococcus_vestibularis, Clostridium_leptum, Klebsiella_aerogenes,
Catenibacterium_mitsuokai, Citrobacter_braakii, Haemophilus_sp_HMSC71H05, Citrobacter_freundii,
Klebsiella_quasipneumoniae, Klebsiella_variicola
PWY4LZ-257: superpathway of fermentation (Chlamydomonas reinhardtii)
Escherichia_coli, Klebsiella_oxytoca, Klebsiella_aerogenes, Klebsiella_pneumoniae, Citrobacter_freundii
PWY66-389: phytol degradation
Enterobacter_cloacae_complex, Hafnia_alvei, Hafnia_paralvei, Escherichia_coli,
Pseudomonas_aeruginosa_group, Morganella_morganii, Klebsiella_oxytoca, Haemophilus_parainfluenzae,
Klebsiella_pneumoniae, Klebsiella_quasipneumoniae
PYRIDNUCSAL-PWY: D salvage pathway I (PNC VI cycle)
Escherichia_coli, Klebsiella_pneumoniae, Citrobacter_amalonaticus, Bifidobacterium_kashiwanohense
RIBOSYN2-PWY: flavin biosynthesis I (bacteria and plants)
Akkermansia_muciniphila, Bacteroides_cellulosilyticus, Bacteroides_dorei, Bacteroides_massiliensis,
Bacteroides_ovatus, Bacteroides_stercoris, Bacteroides_uniformis, Blautia_wexlerae, Ruminococcus_torques,
Dorea_longicatena, Faecalibacterium_prausnitzii, Eubacterium_rectale, Phascolarctobacterium_faecium,
Ruminococcaceae_bacterium_D16, Ruminococcus_callidus, Bacteroides_caccae, Bacteroides_vulgatus,
Bacteroides_xylanisolvens, Barnesiella_intestinihominis, Dorea_formicigenerans, Eubacterium_eligens,
Eubacterium_sp_CAG_38, Odoribacter_splanchnicus, Parabacteroides_distasonis, Roseburia_hominis,
Anaerostipes_hadrus, Bacteroides_fragilis, Blautia_obeum, Citrobacter_amalonaticus, Coprococcus_comes,
Enterobacter_cloacae_complex, Haemophilus_parainfluenzae, Hafnia_paralvei, Clostridium_symbiosum,
Parabacteroides_merdae, Prevotella_copri, Veillonella_atypica, Veillonella_dispar, Veillonella_infantium,
Veillonella_parvula, Clostridium_sp_CAG_167, Escherichia_coli, Eubacterium_ramulus, Roseburia_faecis,
Agathobaculum_butyriciproducens, Bacteroides_finegoldii, Bacteroides_thetaiotaomicron,
Clostridium_bolteae_CAG_59, Clostridium_innocuum, Hungatella_hathewayi, Clostridium_aldenense,
Clostridium_bolteae, Clostridium_sp_CAG_253, Roseburia_intestinalis, Bacteroides_intestinalis,
Butyrivibrio_crossotus, Coprococcus_eutactus, Pseudomonas_aeruginosa_group, Paraprevotella_xylaniphila,
Odoribacter_laneus, Paraprevotella_clara, Anaerotignum_lactatifermentans, Lachnospira_pectinoschiza,
Lactobacillus_rogosae, Bacteroides_galacturonicus, Bacteroides_coprocola, Bacteroides_coprophilus,
Coprobacter_fastidiosus, Dialister_sp_CAG_357, Prevotella_sp_CAG_279, Morganella_morganii,
Bacteroides_eggerthii, Parabacteroides_gordonii, Parabacteroides_sp_CAG_409, Enterococcus_faecium,
Klebsiella_oxytoca, Bilophila_wadsworthia, Coprococcus_catus, Eubacterium_hallii, Desulfovibrio_piger,
Parabacteroides_johnsonii, Bacteroides_faecis, Clostridium_sp_CAG_299, Roseburia_sp_CAG_303,
Acidaminococcus_intestini, Veillonella_rogosae, Clostridium_clostridioforme, Lactobacillus_reuteri,
Phascolarctobacterium_succinatutens, Bacteroides_salyersiae, Bacteroides_clarus, Coprobacter_secundus,
Clostridium_spiroforme, Megamonas_funiformis, Klebsiella_aerogenes, Catenibacterium_mitsuokai,
Parabacteroides_goldsteinii, Eubacterium_sp_CAG_274, Megamonas_hypermegale, Streptococcus_mitis,
Citrobacter_werkmanii, Asaccharobacter_celatus, Fusobacterium_sp_CAG_439, Acinetobacter_ursingii,
Citrobacter_braakii, Haemophilus_sp_HMSC71H05, Acidaminococcus_fermentans, Megasphaera_elsdenii,
Klebsiella_pneumoniae, Prevotella_sp_CAG_873, Bacteroides_nordii, Bacteroides_sartorii,
Anaeromassilibacillus_sp_An250, Flavonifractor_plautii, Blautia_hansenii, Bacteroides_stercorirosoris,
Coprobacillus_cateniformis, Desulfovibrio_fairfieldensis, Veillonella_sp_T11011_6, Bacteroides_sp_OM08_11,
Coprobacter_sp, Intestinibacter_bartlettii, Adlercreutzia_equolifaciens, Lactococcus_lactis,
Pseudoflavonifractor_sp_An184, Dysgonomonas_sp_37_18, Clostridium_sp_CAG_632, Bacteroides_plebeius,
Prevotella_sp_CAG_891, Slackia_isoflavoniconvertens, Bifidobacterium_longum, Citrobacter_youngae,
Streptococcus_pasteurianus, Prevotella_sp_AM42_24, Bacteroides_sp_CAG_443, Holdemanella_biformis,
Ruminococcus_sp_CAG_403, Megasphaera_stantonii, Klebsiella_quasipneumoniae,
Phascolarctobacterium_sp_CAG_266
SO4ASSIM-PWY: assimilatory sulfate reduction I
Escherichia_coli, Citrobacter_amalonaticus, Citrobacter_europaeus, Enterobacter_cloacae_complex,
Enterobacter_mori, Haemophilus_parainfluenzae, Hafnia_alvei, Hafnia_paralvei,
Pseudomonas_aeruginosa_group, Odoribacter_laneus, Klebsiella_oxytoca, Citrobacter_freundii,
Klebsiella_aerogenes, Citrobacter_braakii, Citrobacter_werkmanii, Escherichia_albertii, Klebsiella_pneumoniae,
Klebsiella_michiganensis, Klebsiella_quasipneumoniae, Klebsiella_variicola
SULFATE-CYS-PWY: superpathway of sulfate assimilation and cysteine biosynthesis
Escherichia_coli, Citrobacter_amalonaticus, Citrobacter_europaeus, Enterobacter_cloacae_complex,
Haemophilus_parainfluenzae, Hafnia_alvei, Hafnia_paralvei, Klebsiella_oxytoca, Klebsiella_aerogenes,
Citrobacter_werkmanii, Citrobacter_braakii, Citrobacter_freundii, Klebsiella_pneumoniae,
Klebsiella_quasipneumoniae
TCA: TCA cycle I (prokaryotic)
Escherichia_coli, Enterobacter_cloacae_complex, Enterobacter_mori, Hafnia_alvei, Klebsiella_oxytoca,
Klebsiella_aerogenes, Citrobacter_braakii, Citrobacter_werkmanii, Acinetobacter_ursingii, Citrobacter_youngae,
Hafnia_paralvei, Escherichia_albertii, Klebsiella_pneumoniae, Citrobacter_freundii, Citrobacter_amalonaticus,
Klebsiella_quasipneumoniae, Klebsiella_variicola
TEICHOICACID-PWY: poly(glycerol phosphate) wall teichoic acid biosynthesis
Ruminococcus_torques, Bacteroides_vulgatus, Escherichia_coli, Bacteroides_thetaiotaomicron,
Bacteroides_uniformis, Bacteroides_cellulosilyticus, Parabacteroides_distasonis, Lactobacillus_plantarum
THISYRA-PWY: superpathway of thiamine diphosphate biosynthesis III (eukaryotes)
Bacteroides_ovatus, Bacteroides_uniformis, Ruminococcus_torques, Eubacterium_rectale,
Bacteroides_xylanisolvens, Faecalibacterium_prausnitzii, Roseburia_faecis, Bacteroides_fragilis,
Ruminococcus_gnavus, Dorea_longicatena, Clostridium_aldenense, Roseburia_intestinalis,
Butyrivibrio_crossotus, Roseburia_hominis, Eubacterium_hallii, Bifidobacterium_bifidum,
Clostridium_innocuum, Holdemanella_biformis, Clostridium_scindens, Phascolarctobacterium_succinatutens,
Gordonibacter_pamelaeae, Catenibacterium_mitsuokai, Blautia_coccoides, Hungatella_hathewayi,
Bacteroides_sp_OM08_11, Bacteroides_plebeius, Clostridium_saccharolyticum
Pathway - IBS-D (Pathway: microbe)
ARGININE-SYN4-PWY: L-ornithine biosynthesis II
Bacteroides_dorei, Bacteroides_ovatus, Bacteroides_thetaiotaomicron, Bacteroides_uniformis,
Bacteroides_fragilis, Bacteroides_vulgatus, Bacteroides_caccae, Bacteroides_nordii, Bacteroides_xylanisolvens,
Bacteroides_cellulosilyticus, Bacteroides_stercoris, Paraprevotella_clara, Paraprevotella_xylaniphila,
Bacteroides_massiliensis, Bacteroides_sp_D2, Klebsiella_pneumoniae, Bacteroides_finegoldii,
Bacteroides_coprocola, Bacteroides_plebeius, Bacteroides_intestinalis, Bacteroides_salyersiae,
Bacteroides_sartorii, Catenibacterium_mitsuokai, Citrobacter_freundii, Bacteroides_eggerthii,
Bacteroides_faecis, Bacteroides_faecis_CAG_32, Bacteroides_stercorirosoris, Bacteroides_clarus,
Bacteroides_sp_CAG_462, Bacteroides_coprophilus
ECASYN-PWY: enterobacterial common antigen biosynthesis
Escherichia_coli, Morganella_morganii, Hafnia_paralvei, Proteus_vulgaris, Escherichia_fergusonii,
Klebsiella_pneumoniae, Proteus_hauseri, Enterobacter_cloacae_complex, Proteus_penneri,
Cronobacter_sakazakii, Citrobacter_europaeus, Citrobacter_farmeri, Citrobacter_youngae, Klebsiella_oxytoca,
Klebsiella_variicola, Kluyvera_ascorbata, Citrobacter_werkmanii, Klebsiella_quasipneumoniae,
Citrobacter_freundii
GLUCARDEG-PWY: D-glucarate degradation I
Citrobacter_freundii, Escherichia_coli, Klebsiella_oxytoca, Enterobacter_mori, Escherichia_fergusonii,
Klebsiella_pneumoniae, Enterobacter_cloacae_complex, Citrobacter_europaeus, Citrobacter_farmeri,
Citrobacter_werkmanii, Citrobacter_youngae, Klebsiella_variicola, Kluyvera_cryocrescens,
Clostridium_clostridioforme, Klebsiella_quasipneumoniae, Clostridium_aldenense
GLUCUROCAT-PWY: superpathway of &beta;-D-glucuronosides degradation
Faecalibacterium_prausnitzii, Bacteroides_fragilis, Citrobacter_freundii, Escherichia_coli,
Escherichia_fergusonii, Klebsiella_pneumoniae, Cronobacter_sakazakii, Citrobacter_europaeus,
Citrobacter_youngae, Klebsiella_oxytoca, Lactobacillus_rhamnosus, Kluyvera_ascorbata,
Intestinimonas_butyriciproducens, Citrobacter_werkmanii
GLYCOLYSIS: glycolysis I (from glucose 6-phosphate)
Citrobacter_freundii, Enterobacter_cloacae_complex, Bacteroides_nordii, Bacteroides_fragilis, Escherichia_coli,
Morganella_morganii, Citrobacter_braakii, Hafnia_paralvei, Enterobacter_mori, Escherichia_fergusonii,
Klebsiella_pneumoniae, Klebsiella_quasipneumoniae, Klebsiella_variicola, Proteus_hauseri,
Streptococcus_gordonii, Streptococcus_salivarius, Bacteroides_salyersiae, Proteus_penneri,
Cronobacter_sakazakii, Haemophilus_parainfluenzae, Citrobacter_europaeus, Citrobacter_farmeri,
Citrobacter_werkmanii, Citrobacter_youngae, Klebsiella_oxytoca, Kluyvera_cryocrescens,
Lactobacillus_rhamnosus, Proteus_vulgaris, Kluyvera_ascorbata, Raoultella_planticola,
Bacteroides_cellulosilyticus, Catenibacterium_mitsuokai, Parabacteroides_distasonis, Klebsiella_michiganensis,
Bacteroides_xylanisolvens, Citrobacter_portucalensis
GOLPDLCAT-PWY: superpathway of glycerol degradation to 1,3-propanediol
Anaerostipes_hadrus, Ruminococcus_torques, Citrobacter_freundii, Enterobacter_cloacae_complex,
Citrobacter_braakii, Hafnia_paralvei, Escherichia_coli, Klebsiella_pneumoniae, Klebsiella_variicola,
Flavonifractor_plautii, Citrobacter_europaeus, Citrobacter_werkmanii, Citrobacter_youngae, Klebsiella_oxytoca,
Eubacterium_hallii, Klebsiella_quasipneumoniae
HISTSYN-PWY: L-histidine biosynthesis
Bacteroides_dorei, Bacteroides_fragilis, Bacteroides_ovatus, Bacteroides_thetaiotaomicron,
Bacteroides_uniformis, Barnesiella_intestinihominis, Blautia_obeum, Ruminococcus_torques,
Dorea_formicigenerans, Faecalibacterium_prausnitzii, Odoribacter_splanchnicus, Parabacteroides_distasonis,
Parasutterella_excrementihominis, Eubacterium_siraeum, Bacteroides_vulgatus, Citrobacter_freundii,
Parabacteroides_merdae, Roseburia_intestinalis, Bacteroides_caccae, Bacteroides_nordii,
Bacteroides_xylanisolvens, Roseburia_inulinivorans, Ruminococcus_bromii, Bacteroides_cellulosilyticus,
Bacteroides_stercoris, Escherichia_coli, Morganella_morganii, Parabacteroides_johnsonii, Paraprevotella_clara,
Bacteroides_massiliensis, Coprococcus_catus, Hafnia_paralvei, Klebsiella_oxytoca, Bacteroides_sp_D2,
Escherichia_fergusonii, Klebsiella_pneumoniae, Klebsiella_quasipneumoniae, Clostridium_clostridioforme,
Veillonella_parvula, Bifidobacterium_adolescentis, Eubacterium_sp_CAG_180, Lactococcus_lactis,
Bacteroides_finegoldii, Bacteroides_sp_CAG_633, Collinsella_aerofaciens, Eubacterium_sp_CAG_251,
Dialister_sp_CAG_357, Enterobacter_cloacae_complex, Bacteroides_coprocola, Bacteroides_plebeius,
Coprobacter_fastidiosus, Alistipes_finegoldii, Alistipes_shahii, Bacteroides_intestinalis, Bacteroides_salyersiae,
Bacteroides_sartorii, Flavonifractor_plautii, Lawsonibacter_asaccharolyticus, Bifidobacterium_bifidum,
Bifidobacterium_longum, Eubacterium_sp_CAG_38, Clostridium_leptum, Eggerthella_lenta,
Eisenbergiella_massiliensis, Collinsella_stercoris, Megasphaera_sp_DISK_18,
Proteobacteria_bacterium_CAG_139, Cronobacter_sakazakii, Gemmiger_sp_An87,
Agathobaculum_butyriciproducens, Bacteroides_eggerthii, Bacteroides_sp_CAG_144,
Bifidobacterium_pseudocatenulatum, Prevotella_sp_CAG_1320, Clostridium_spiroforme,
Eubacterium_sp_CAG_274, Firmicutes_bacterium_CAG_424, Streptococcus_thermophilus,
Bifidobacterium_breve, Citrobacter_europaeus, Citrobacter_farmeri, Klebsiella_variicola,
Kluyvera_cryocrescens, Lactobacillus_rhamnosus, Paenibacillus_thiaminolyticus, Proteus_vulgaris,
Saccharomyces_cerevisiae, Paraprevotella_xylaniphila, Prevotella_sp_CAG_279, Prevotella_copri,
Kluyvera_ascorbata, Lactobacillus_ruminis, Catenibacterium_mitsuokai, Mitsuokella_multacida,
Roseburia_hominis, Clostridium_sp_CAG_678, Ruminococcaceae_bacterium_D16,
Ruthenibacterium_lactatiformans, Butyricimonas_virosa, Methanobrevibacter_smithii, Citrobacter_youngae,
Clostridium_bolteae, Prevotella_sp_AM42_24, Bacteroides_faecis, Bacteroides_faecis_CAG_32,
Clostridium_disporicum, Streptococcus_salivarius, Bacteroides_stercorirosoris, Eubacterium_ventriosum,
Bifidobacterium_animalis, Lactobacillus_plantarum, Alistipes_onderdonkii, Parabacteroides_gordonii,
Bacteroides_clarus, Anaerostipes_caccae, Erysipelatoclostridium_ramosum, Citrobacter_werkmanii,
Clostridium_bolteae_CAG_59, Coprobacillus_cateniformis, Enterococcus_casseliflavus, Clostridium_innocuum,
Anaerotignum_lactatifermentans, Odoribacter_laneus, Alistipes_indistinctus, Parabacteroides_goldsteinii,
Ruminococcus_sp_CAG_330, Bifidobacterium_pullorum, Bifidobacterium_saeculare, Hungatella_hathewayi,
Clostridium_aldenense, Acidaminococcus_fermentans, Bacteroides_sp_CAG_462,
Parabacteroides_sp_CAG_409, Prevotella_sp_CAG_1185, Megasphaera_sp_MJR8396C, Megasphaera_elsdenii,
Prevotella_sp_CAG_1092, Intestinimonas_butyriciproducens, Blautia_producta,
Firmicutes_bacterium_CAG_94, Megamonas_funiformis
KETOGLUCONMET-PWY: ketogluconate metabolism
Escherichia_coli, Hafnia_paralvei, Klebsiella_oxytoca, Escherichia_fergusonii, Klebsiella_pneumoniae,
Enterobacter_cloacae_complex, Proteus_penneri, Cronobacter_sakazakii, Citrobacter_farmeri,
Citrobacter_youngae, Klebsiella_variicola, Proteus_vulgaris, Citrobacter_freundii, Lactobacillus_plantarum,
Citrobacter_koseri, Bifidobacterium_gallinarum, Bifidobacterium_pullorum
METH-ACETATE-PWY: methanogenesis from acetate
Blautia_obeum, Blautia_wexlerae, Ruminococcus_torques, Fusicatenibacter_saccharivorans,
Blautia_sp_CAG_257, Eubacterium_limosum
GLIPASYN-PWY: lipid IVA biosynthesis (E. coli)
Bacteroides_fragilis, Bacteroides_uniformis, Parasutterella_excrementihominis, Citrobacter_freundii,
Bacteroides_stercoris, Escherichia_coli, Morganella_morganii, Hafnia_paralvei, Proteus_vulgaris,
Escherichia_fergusonii, Klebsiella_pneumoniae, Klebsiella_quasipneumoniae, Proteus_hauseri,
Bacteroides_sp_CAG_633, Enterobacter_cloacae_complex, Proteobacteria_bacterium_CAG_139,
Bacteroides_intestinalis, Bacteroides_cellulosilyticus, Proteus_penneri, Cronobacter_sakazakii,
Haemophilus_parainfluenzae, Citrobacter_europaeus, Citrobacter_farmeri, Citrobacter_werkmanii,
Citrobacter_youngae, Klebsiella_oxytoca, Klebsiella_variicola, Kluyvera_cryocrescens, Kluyvera_ascorbata,
Raoultella_planticola, Catenibacterium_mitsuokai, Citrobacter_portucalensis
P124-PWY: Bifidobacterium shunt
Bifidobacterium_longum, Collinsella_aerofaciens, Bifidobacterium_dentium,
Bifidobacterium_pseudocatenulatum
P161-PWY: acetylene degradation (anaerobic)
Citrobacter_freundii, Enterobacter_cloacae_complex, Escherichia_coli, Morganella_morganii,
Citrobacter_braakii, Hafnia_paralvei, Atlantibacter_hermannii, Enterobacter_mori, Escherichia_fergusonii,
Fusobacterium_nucleatum, Klebsiella_pneumoniae, Klebsiella_quasipneumoniae, Klebsiella_variicola,
Streptococcus_gordonii, Flavonifractor_plautii, Proteus_penneri, Cronobacter_sakazakii,
Bifidobacterium_animalis, Citrobacter_europaeus, Citrobacter_farmeri, Citrobacter_werkmanii,
Citrobacter_youngae, Klebsiella_oxytoca, Kluyvera_cryocrescens, Kluyvera_ascorbata, Raoultella_planticola,
Lactobacillus_plantarum, Klebsiella_michiganensis, Citrobacter_portucalensis, Hungatella_hathewayi,
Clostridium_aldenense
P221-PWY: octane oxidation
Escherichia_coli, Klebsiella_pneumoniae, Klebsiella_quasipneumoniae, Enterobacter_cloacae_complex,
Cronobacter_sakazakii, Kluyvera_ascorbata, Klebsiella_oxytoca
PENTOSE-P-PWY: pentose phosphate pathway
Parabacteroides_distasonis, Bacteroides_fragilis, Citrobacter_freundii, Enterobacter_cloacae_complex,
Bacteroides_xylanisolvens, Bacteroides_cellulosilyticus, Escherichia_coli, Morganella_morganii,
Citrobacter_braakii, Hafnia_paralvei, Klebsiella_oxytoca, Enterobacter_mori, Escherichia_fergusonii,
Klebsiella_pneumoniae, Klebsiella_quasipneumoniae, Clostridium_clostridioforme, Bifidobacterium_bifidum,
Bacteroides_thetaiotaomicron, Lactobacillus_salivarius, Cronobacter_sakazakii, Haemophilus_parainfluenzae,
Citrobacter_europaeus, Citrobacter_farmeri, Citrobacter_werkmanii, Citrobacter_youngae, Klebsiella_variicola,
Kluyvera_cryocrescens, Lactobacillus_rhamnosus, Saccharomyces_cerevisiae, Lactobacillus_plantarum,
Citrobacter_portucalensis, Hungatella_hathewayi, Clostridium_aldenense
PHOSLIPSYN-PWY: superpathway of phospholipid biosynthesis I (bacteria)
Fusicatenibacter_saccharivorans, Escherichia_coli, Morganella_morganii, Hafnia_paralvei, Klebsiella_oxytoca,
Ruminococcus_gnavus, Escherichia_fergusonii, Klebsiella_pneumoniae, Proteus_hauseri,
Enterobacter_cloacae_complex, Proteus_penneri, Clostridium_leptum, Cronobacter_sakazakii,
Haemophilus_parainfluenzae, Citrobacter_europaeus, Citrobacter_farmeri, Citrobacter_youngae,
Klebsiella_variicola, Kluyvera_cryocrescens, Proteus_vulgaris, Kluyvera_ascorbata, Catenibacterium_mitsuokai,
Citrobacter_freundii, Fusobacterium_mortiferum, Citrobacter_werkmanii, Klebsiella_michiganensis,
Klebsiella_quasipneumoniae
POLYISOPRENSYN-PWY: polyisoprenoid biosynthesis (E. coli)
Citrobacter_freundii, Enterobacter_cloacae_complex, Escherichia_coli, Morganella_morganii,
Citrobacter_braakii, Hafnia_paralvei, Klebsiella_oxytoca, Escherichia_fergusonii, Klebsiella_pneumoniae,
Citrobacter_farmeri, Citrobacter_werkmanii, Citrobacter_youngae, Klebsiella_variicola,
Klebsiella_quasipneumoniae
PWY-4321: L-glutamate degradation IV
Escherichia_coli, Hafnia_paralvei, Klebsiella_pneumoniae
PWY-5030: L-histidine degradation III
Alistipes_putredinis, Bacteroides_dorei, Bacteroides_fragilis, Bacteroides_ovatus, Bacteroides_thetaiotaomicron,
Bacteroides_uniformis, Bacteroides_vulgatus, Alistipes_shahii, Bacteroides_xylanisolvens,
Bacteroides_stercoris, Streptococcus_parasanguinis, Bacteroides_faecis, Bacteroides_massiliensis,
Bacteroides_sp_D2, Fusobacterium_nucleatum, Streptococcus_gordonii, Streptococcus_salivarius,
Streptococcus_sanguinis, Bacteroides_sartorii, Alistipes_finegoldii, Bacteroides_eggerthii,
Catenibacterium_mitsuokai, Alistipes_onderdonkii, Bacteroides_clarus, Acidaminococcus_fermentans,
Clostridium_saccharolyticum
PWY-5083: D(P)/DPH interconversion
Citrobacter_freundii, Enterobacter_cloacae_complex, Escherichia_coli, Citrobacter_braakii, Enterobacter_mori,
Escherichia_fergusonii, Klebsiella_pneumoniae, Klebsiella_quasipneumoniae, Klebsiella_variicola,
Citrobacter_europaeus, Citrobacter_farmeri, Citrobacter_werkmanii, Citrobacter_youngae, Klebsiella_oxytoca,
Kluyvera_cryocrescens, Saccharomyces_cerevisiae, Kluyvera_ascorbata, Raoultella_planticola,
Citrobacter_koseri, Citrobacter_portucalensis
PWY-5104: L-isoleucine biosynthesis IV
Parabacteroides_distasonis, Citrobacter_freundii, Parabacteroides_merdae, Escherichia_coli, Citrobacter_braakii,
Escherichia_fergusonii, Klebsiella_pneumoniae, Klebsiella_variicola, Parabacteroides_goldsteinii,
Eggerthella_lenta, Dorea_longicatena, Citrobacter_europaeus, Citrobacter_farmeri, Citrobacter_werkmanii,
Citrobacter_youngae, Klebsiella_oxytoca, Kluyvera_ascorbata, Klebsiella_quasipneumoniae,
Citrobacter_portucalensis
PWY-5189: tetrapyrrole biosynthesis II (from glycine)
Escherichia_coli, Proteus_vulgaris, Escherichia_fergusonii, Klebsiella_pneumoniae, Proteus_hauseri,
Enterobacter_cloacae_complex, Proteus_penneri, Cronobacter_sakazakii, Citrobacter_farmeri,
Klebsiella_oxytoca, Saccharomyces_cerevisiae, Romboutsia_ilealis, Clostridium_perfringens,
Intestinimonas_butyriciproducens
PWY-5345: superpathway of L-methionine biosynthesis (by sulfhydrylation)
Klebsiella_pneumoniae, Proteus_penneri, Saccharomyces_cerevisiae
PWY-5484: glycolysis II (from fructose 6-phosphate)
Citrobacter_freundii, Enterobacter_cloacae_complex, Bacteroides_nordii, Bacteroides_fragilis, Escherichia_coli,
Morganella_morganii, Citrobacter_braakii, Enterobacter_mori, Escherichia_fergusonii, Klebsiella_pneumoniae,
Klebsiella_quasipneumoniae, Klebsiella_variicola, Proteus_hauseri, Bacteroides_salyersiae, Proteus_penneri,
Hafnia_paralvei, Cronobacter_sakazakii, Haemophilus_parainfluenzae, Citrobacter_europaeus,
Citrobacter_farmeri, Citrobacter_werkmanii, Citrobacter_youngae, Klebsiella_oxytoca, Kluyvera_cryocrescens,
Lactobacillus_rhamnosus, Proteus_vulgaris, Kluyvera_ascorbata, Raoultella_planticola,
Bacteroides_cellulosilyticus, Catenibacterium_mitsuokai, Parabacteroides_distasonis, Klebsiella_michiganensis,
Bacteroides_xylanisolvens, Citrobacter_portucalensis
PWY-5675: nitrate reduction V (assimilatory)
Escherichia_coli, Escherichia_fergusonii, Klebsiella_pneumoniae, Klebsiella_quasipneumoniae,
Klebsiella_variicola, Enterobacter_cloacae_complex, Citrobacter_europaeus, Klebsiella_oxytoca,
Kluyvera_cryocrescens, Kluyvera_ascorbata, Raoultella_planticola, Citrobacter_koseri
PWY-5695: inosine 5′-phosphate degradation
Alistipes_putredinis, Anaerostipes_hadrus, Bacteroides_dorei, Bacteroides_fragilis, Bacteroides_ovatus,
Bacteroides_thetaiotaomicron, Bacteroides_uniformis, Barnesiella_intestinihominis, Blautia_obeum,
Blautia_wexlerae, Ruminococcus_torques, Coprococcus_comes, Dorea_longicatena, Eubacterium_eligens,
Faecalibacterium_prausnitzii, Fusicatenibacter_saccharivorans, Lachnospira_pectinoschiza,
Parabacteroides_distasonis, Bacteroides_vulgatus, Citrobacter_freundii, Citrobacter_youngae,
Enterobacter_cloacae_complex, Roseburia_intestinalis, Bacteroides_caccae, Bacteroides_nordii,
Bacteroides_xylanisolvens, Roseburia_faecis, Roseburia_inulinivorans, Ruminococcus_bicirculans,
Bacteroides_stercoris, Escherichia_coli, Morganella_morganii, Parabacteroides_johnsonii,
Streptococcus_salivarius, Bacteroides_faecis, Bacteroides_faecis_CAG_32, Bacteroides_massiliensis,
Coprococcus_eutactus, Hafnia_paralvei, Klebsiella_oxytoca, Proteus_vulgaris, Bacteroides_sp_D2,
Dialister_invisus, Enterobacter_mori, Escherichia_fergusonii, Klebsiella_pneumoniae,
Klebsiella_quasipneumoniae, Klebsiella_variicola, Clostridium_clostridioforme, Proteus_hauseri,
Streptococcus_mitis, Streptococcus_oralis, Streptococcus_vestibularis, Veillonella_parvula,
Eubacterium_ramulus, Bacteroides_cellulosilyticus, Bacteroides_finegoldii, Bacteroides_sp_CAG_633,
Bacteroides_galacturonicus, Eubacterium_ventriosum, Intestinimonas_butyriciproducens,
Bilophila_wadsworthia, Hungatella_hathewayi, Bacteroides_coprocola, Prevotella_sp_CAG_1031,
Bacteroides_plebeius, Lactobacillus_rogosae, Odoribacter_splanchnicus, Alistipes_shahii,
Bacteroides_intestinalis, Bacteroides_salyersiae, Bacteroides_sartorii, Proteus_penneri, Alistipes_finegoldii,
Agathobaculum_butyriciproducens, Eisenbergiella_massiliensis, Megasphaera_sp_DISK_18,
Cronobacter_sakazakii, Haemophilus_parainfluenzae, Bacteroides_eggerthii, Coprococcus_catus,
Streptococcus_sp_A12, Clostridiales_bacterium_1_7_47FAA, Citrobacter_europaeus, Citrobacter_farmeri,
Citrobacter_werkmanii, Kluyvera_cryocrescens, Lactobacillus_rhamnosus, Paenibacillus_thiaminolyticus,
Prevotella_sp_CAG_279, Prevotella_copri, Kluyvera_ascorbata, Raoultella_planticola, Mitsuokella_multacida,
Coprobacter_fastidiosus, Clostridium_symbiosum, Alistipes_sp_An31A, Catenibacterium_mitsuokai,
Coprobacter_sp, Eisenbergiella_tayi, Clostridium_bolteae, Prevotella_sp_AM42_24, Turicibacter_sanguinis,
Clostridium_disporicum, Intestinibacter_bartlettii, Romboutsia_ilealis, Streptococcus_thermophilus,
Bacteroides_stercorirosoris, Clostridium_perfringens, Clostridium_citroniae, Alistipes_onderdonkii,
Parabacteroides_gordonii, Flavonifractor_plautii, Firmicutes_bacterium_CAG_94, Clostridium_sp_7_2_43FAA,
Megasphaera_micronuciformis, Terrisporobacter_othiniensis, Clostridium_aldenense, Alistipes_indistinctus,
Citrobacter_koseri, Citrobacter_portucalensis, Parabacteroides_goldsteinii, Bacteroides_coprophilus,
Bacteroides_sp_CAG_462, Megasphaera_sp_MJR8396C, Desulfovibrio_piger, Megasphaera_elsdenii,
Clostridium_saccharolyticum, Alistipes_inops, Bacteroides_clarus
PWY-5723: Rubisco shunt
Citrobacter_freundii, Escherichia_coli, Citrobacter_braakii, Hafnia_paralvei, Escherichia_fergusonii,
Klebsiella_pneumoniae, Enterobacter_cloacae_complex, Cronobacter_sakazakii, Citrobacter_farmeri,
Citrobacter_youngae, Klebsiella_oxytoca, Klebsiella_variicola, Kluyvera_cryocrescens, Citrobacter_werkmanii,
Klebsiella_michiganensis, Klebsiella_quasipneumoniae, Citrobacter_portucalensis
PWY-5918: superpathway of heme b biosynthesis from glutamate
Escherichia_coli, Morganella_morganii, Citrobacter_braakii, Escherichia_fergusonii, Klebsiella_pneumoniae,
Proteus_hauseri, Enterobacter_cloacae_complex, Proteus_penneri, Cronobacter_sakazakii, Citrobacter_farmeri,
Citrobacter_youngae, Klebsiella_oxytoca, Klebsiella_variicola
PWY-5971: palmitate biosynthesis (type II fatty acid synthase)
Citrobacter_freundii, Escherichia_coli, Citrobacter_braakii, Enterobacter_mori, Escherichia_fergusonii,
Klebsiella_pneumoniae, Enterobacter_cloacae_complex, Citrobacter_farmeri, Citrobacter_werkmanii,
Citrobacter_youngae, Klebsiella_oxytoca, Citrobacter_portucalensis
PWY-622: starch biosynthesis
Bifidobacterium_gallinarum, Bifidobacterium_pullorum, Klebsiella_oxytoca
PWY-6284: superpathway of unsaturated fatty acids biosynthesis (E. coli)
Citrobacter_freundii, Escherichia_coli, Citrobacter_braakii, Enterobacter_mori, Escherichia_fergusonii,
Klebsiella_pneumoniae, Streptococcus_oralis, Enterobacter_cloacae_complex, Citrobacter_farmeri,
Citrobacter_werkmanii, Citrobacter_youngae, Klebsiella_oxytoca, Citrobacter_portucalensis
PWY-6507: 4-deoxy-L-threo-hex-4-enopyranuronate degradation
Faecalibacterium_prausnitzii, Citrobacter_freundii, Enterobacter_cloacae_complex, Escherichia_coli,
Morganella_morganii, Citrobacter_braakii, Hafnia_paralvei, Klebsiella_oxytoca, Klebsiella_pneumoniae,
Hungatella_hathewayi, Clostridium_clostridioforme, Cronobacter_sakazakii,
Clostridiales_bacterium_1_7_47FAA, Clostridium_bolteae, Citrobacter_europaeus, Citrobacter_farmeri,
Citrobacter_werkmanii, Citrobacter_youngae, Kluyvera_cryocrescens, Lactobacillus_rhamnosus,
Kluyvera_ascorbata, Turicibacter_sanguinis, Citrobacter_portucalensis, Clostridium_aldenense
PWY-6545: pyrimidine deoxyribonucleotides de novo biosynthesis III
Escherichia_coli, Klebsiella_pneumoniae, Citrobacter_farmeri, Klebsiella_oxytoca, Klebsiella_variicola,
Klebsiella_quasipneumoniae
PWY-6803: phosphatidylcholine acyl editing
Enterobacter_cloacae_complex, Escherichia_coli, Morganella_morganii, Citrobacter_braakii, Hafnia_paralvei,
Klebsiella_oxytoca, Escherichia_fergusonii, Klebsiella_pneumoniae, Klebsiella_quasipneumoniae,
Klebsiella_variicola, Proteus_penneri, Citrobacter_europaeus, Citrobacter_farmeri, Citrobacter_youngae,
Kluyvera_cryocrescens, Kluyvera_ascorbata, Raoultella_planticola, Citrobacter_freundii, Citrobacter_koseri,
Citrobacter_werkmanii, Klebsiella_michiganensis
PWY-7184: pyrimidine deoxyribonucleotides de novo biosynthesis I
Citrobacter_freundii, Escherichia_coli, Morganella_morganii, Hafnia_paralvei, Klebsiella_oxytoca,
Escherichia_fergusonii, Klebsiella_pneumoniae, Klebsiella_quasipneumoniae, Proteus_hauseri, Proteus_penneri,
Haemophilus_parainfluenzae, Citrobacter_europaeus, Citrobacter_farmeri, Citrobacter_youngae,
Klebsiella_variicola, Kluyvera_cryocrescens, Lactobacillus_rhamnosus, Kluyvera_ascorbata,
Citrobacter_werkmanii
PWY-7204: pyridoxal 5′-phosphate salvage II (plants)
Citrobacter_freundii, Escherichia_coli, Escherichia_fergusonii, Klebsiella_pneumoniae,
Enterobacter_cloacae_complex, Citrobacter_europaeus, Citrobacter_farmeri, Citrobacter_youngae,
Klebsiella_oxytoca, Citrobacter_werkmanii
PWY-7210: pyrimidine deoxyribonucleotides biosynthesis from CTP
Escherichia_coli, Morganella_morganii, Hafnia_paralvei, Klebsiella_pneumoniae, Klebsiella_quasipneumoniae,
Proteus_hauseri, Proteus_penneri, Haemophilus_parainfluenzae, Citrobacter_farmeri, Klebsiella_oxytoca,
Klebsiella_variicola, Lactobacillus_rhamnosus
PWY-7211: superpathway of pyrimidine deoxyribonucleotides de novo biosynthesis
Escherichia_coli, Klebsiella_pneumoniae, Citrobacter_farmeri, Klebsiella_oxytoca, Klebsiella_variicola,
Klebsiella_quasipneumoniae
PWY-7242: D-fructuronate degradation
Faecalibacterium_prausnitzii, Bacteroides_fragilis, Citrobacter_freundii, Enterobacter_cloacae_complex,
Escherichia_coli, Morganella_morganii, Citrobacter_braakii, Hafnia_paralvei, Klebsiella_oxytoca,
Escherichia_fergusonii, Klebsiella_pneumoniae, Intestinimonas_butyriciproducens, Cronobacter_sakazakii,
Clostridium_sp_CAG_58, Lactococcus_lactis, Clostridiales_bacterium_1_7_47FAA, Citrobacter_europaeus,
Citrobacter_farmeri, Citrobacter_werkmanii, Citrobacter_youngae, Kluyvera_cryocrescens,
Lactobacillus_rhamnosus, Kluyvera_ascorbata, Citrobacter_portucalensis, Clostridium_aldenense,
Dorea_longicatena
PWY-7269: mitochondrial DPH production (yeast)
Escherichia_coli, Escherichia_fergusonii, Klebsiella_pneumoniae, Klebsiella_quasipneumoniae,
Enterobacter_cloacae_complex, Cronobacter_sakazakii, Klebsiella_oxytoca, Klebsiella_variicola,
Kluyvera_cryocrescens, Saccharomyces_cerevisiae, Kluyvera_ascorbata, Raoultella_planticola,
Citrobacter_portucalensis, Citrobacter_youngae
PWY-7282: 4-amino-2-methyl-5-diphosphomethylpyrimidine biosynthesis II
Bacteroides_fragilis, Bacteroides_ovatus, Bacteroides_uniformis, Bacteroides_xylanisolvens, Escherichia_coli,
Streptococcus_salivarius, Citrobacter_braakii, Bacteroides_sp_D2, Escherichia_fergusonii,
Klebsiella_pneumoniae, Klebsiella_quasipneumoniae, Streptococcus_vestibularis,
Enterobacter_cloacae_complex, Citrobacter_europaeus, Citrobacter_farmeri, Citrobacter_youngae,
Klebsiella_oxytoca, Klebsiella_variicola, Kluyvera_cryocrescens, Saccharomyces_cerevisiae,
Kluyvera_ascorbata, Catenibacterium_mitsuokai, Citrobacter_freundii, Haemophilus_parainfluenzae,
Citrobacter_werkmanii, Klebsiella_michiganensis
PWY-7392: taxadiene biosynthesis (engineered)
Hafnia_paralvei, Klebsiella_pneumoniae
PWY-7446: sulfoquinovose degradation I
Escherichia_coli, Escherichia_fergusonii, Klebsiella_pneumoniae, Enterobacter_cloacae_complex,
Cronobacter_sakazakii, Citrobacter_europaeus, Citrobacter_farmeri, Citrobacter_youngae, Klebsiella_oxytoca,
Raoultella_planticola, Citrobacter_freundii, Citrobacter_werkmanii, Citrobacter_portucalensis
PWY-7456: &beta;-(1,4)-mannan degradation
Eubacterium_siraeum, Roseburia_faecis, Coprococcus_eutactus, Clostridium_disporicum
PWY0-1298: superpathway of pyrimidine deoxyribonucleosides degradation
Citrobacter_youngae, Enterobacter_cloacae_complex, Escherichia_coli, Morganella_morganii,
Citrobacter_braakii, Hafnia_paralvei, Escherichia_fergusonii, Klebsiella_pneumoniae,
Klebsiella_quasipneumoniae, Klebsiella_variicola, Streptococcus_mitis, Streptococcus_oralis,
Hungatella_hathewayi, Streptococcus_sp_M334, Cronobacter_sakazakii, Lactococcus_lactis,
Citrobacter_europaeus, Citrobacter_farmeri, Citrobacter_werkmanii, Klebsiella_oxytoca, Kluyvera_cryocrescens,
Kluyvera_ascorbata, Raoultella_planticola, Citrobacter_freundii, Clostridium_baratii, Clostridium_perfringens,
Terrisporobacter_othiniensis, Citrobacter_portucalensis
PWY30-355: stearate biosynthesis III (fungi)
Bacteroides_dorei, Bacteroides_uniformis, Bacteroides_vulgatus, Saccharomyces_cerevisiae,
Bifidobacterium_longum
PWY4FS-7: phosphatidylglycerol biosynthesis I (plastidic)
Fusicatenibacter_saccharivorans, Escherichia_coli, Morganella_morganii, Streptococcus_salivarius,
Hafnia_paralvei, Klebsiella_oxytoca, Ruminococcus_gnavus, Escherichia_fergusonii, Klebsiella_pneumoniae,
Klebsiella_quasipneumoniae, Proteus_hauseri, Streptococcus_vestibularis, Enterobacter_cloacae_complex,
Faecalibacterium_prausnitzii, Streptococcus_thermophilus, Proteus_penneri, Roseburia_intestinalis,
Clostridium_leptum, Cronobacter_sakazakii, Haemophilus_parainfluenzae, Citrobacter_europaeus,
Citrobacter_farmeri, Citrobacter_youngae, Klebsiella_variicola, Kluyvera_cryocrescens, Proteus_vulgaris,
Kluyvera_ascorbata, Catenibacterium_mitsuokai, Citrobacter_freundii, Turicibacter_sanguinis,
Fusobacterium_mortiferum, Citrobacter_werkmanii
PWY4FS-8: phosphatidylglycerol biosynthesis II (non-plastidic)
Fusicatenibacter_saccharivorans, Escherichia_coli, Morganella_morganii, Streptococcus_salivarius,
Hafnia_paralvei, Klebsiella_oxytoca, Ruminococcus_gnavus, Escherichia_fergusonii, Klebsiella_pneumoniae,
Klebsiella_quasipneumoniae, Proteus_hauseri, Streptococcus_vestibularis, Enterobacter_cloacae_complex,
Faecalibacterium_prausnitzii, Streptococcus_thermophilus, Proteus_penneri, Roseburia_intestinalis,
Clostridium_leptum, Cronobacter_sakazakii, Haemophilus_parainfluenzae, Citrobacter_europaeus,
Citrobacter_farmeri, Citrobacter_youngae, Klebsiella_variicola, Kluyvera_cryocrescens, Proteus_vulgaris,
Kluyvera_ascorbata, Catenibacterium_mitsuokai, Citrobacter_freundii, Turicibacter_sanguinis,
Fusobacterium_mortiferum, Citrobacter_werkmanii
PWY4LZ-257: superpathway of fermentation (Chlamydomonas reinhardtii)
Escherichia_coli, Klebsiella_pneumoniae, Cronobacter_sakazakii, Klebsiella_oxytoca, Citrobacter_freundii
PWY66-389: phytol degradation
Escherichia_coli, Morganella_morganii, Hafnia_paralvei, Proteus_vulgaris, Klebsiella_pneumoniae,
Klebsiella_quasipneumoniae, Proteus_hauseri, Enterobacter_cloacae_complex, Proteus_penneri,
Cronobacter_sakazakii, Haemophilus_parainfluenzae, Klebsiella_oxytoca, Saccharomyces_cerevisiae,
Kluyvera_ascorbata
PYRIDNUCSAL-PWY: D salvage pathway I (PNC VI cycle)
Escherichia_coli, Hafnia_paralvei, Proteus_vulgaris, Klebsiella_pneumoniae, Escherichia_fergusonii,
Proteus_penneri, Citrobacter_farmeri, Klebsiella_oxytoca
RIBOSYN2-PWY: flavin biosynthesis I (bacteria and plants)
Agathobaculum_butyriciproducens, Anaerostipes_hadrus, Bacteroides_dorei, Bacteroides_fragilis,
Bacteroides_ovatus, Bacteroides_thetaiotaomicron, Bacteroides_uniformis, Barnesiella_intestinihominis,
Blautia_obeum, Blautia_wexlerae, Ruminococcus_torques, Dorea_formicigenerans, Dorea_longicatena,
Eubacterium_eligens, Eubacterium_hallii, Faecalibacterium_prausnitzii, Lachnospira_pectinoschiza,
Eubacterium_rectale, Odoribacter_splanchnicus, Parabacteroides_distasonis, Phascolarctobacterium_faecium,
Bacteroides_vulgatus, Parabacteroides_merdae, Bacteroides_caccae, Bacteroides_nordii,
Bacteroides_xylanisolvens, Roseburia_faecis, Roseburia_hominis, Bacteroides_cellulosilyticus,
Bacteroides_stercoris, Escherichia_coli, Morganella_morganii, Parabacteroides_johnsonii,
Bacteroides_massiliensis, Citrobacter_braakii, Coprococcus_catus, Coprococcus_comes, Coprococcus_eutactus,
Clostridium_spiroforme, Hafnia_paralvei, Klebsiella_oxytoca, Proteus_vulgaris, Bacteroides_sp_D2,
Escherichia_fergusonii, Klebsiella_pneumoniae, Clostridium_clostridioforme, Proteus_hauseri,
Veillonella_parvula, Dialister_sp_CAG_357, Ruminococcus_callidus, Bilophila_wadsworthia,
Akkermansia_muciniphila, Bacteroides_galacturonicus, Enterobacter_cloacae_complex, Bacteroides_finegoldii,
Prevotella_sp_CAG_1031, Lactobacillus_rogosae, Paraprevotella_clara, Bacteroides_coprocola,
Bacteroides_intestinalis, Bacteroides_salyersiae, Bacteroides_sartorii, Roseburia_intestinalis,
Ruminococcaceae_bacterium_D16, Proteus_penneri, Anaeromassilibacillus_sp_An250, Eubacterium_ramulus,
Eubacterium_sp_CAG_38, Adlercreutzia_equolifaciens, Asaccharobacter_celatus, Cronobacter_sakazakii,
Haemophilus_parainfluenzae, Lactococcus_lactis, Citrobacter_farmeri, Citrobacter_werkmanii,
Citrobacter_youngae, Fusobacterium_ulcerans, Klebsiella_variicola, Kluyvera_cryocrescens,
Prevotella_sp_CAG_279, Paraprevotella_xylaniphila, Prevotella_copri, Kluyvera_ascorbata,
Holdemanella_biformis, Mitsuokella_multacida, Slackia_isoflavoniconvertens, Coprobacter_fastidiosus,
Dialister_pneumosintes, Clostridium_symbiosum, Butyrivibrio_crossotus, Catenibacterium_mitsuokai,
Clostridium_sp_CAG_167, Citrobacter_freundii, Bacteroides_eggerthii, Prevotella_sp_AM42_24,
Bacteroides_faecis, Acidaminococcus_intestini, Clostridium_disporicum, Intestinibacter_bartlettii,
Bacteroides_stercorirosoris, Phascolarctobacterium_succinatutens, Ruminococcus_sp_CAG_330,
Clostridium_perfringens, Anaerotignum_lactatifermentans, Parabacteroides_gordonii,
Fusobacterium_mortiferum, Megamonas_funiformis, Anaerostipes_caccae, Clostridium_bolteae_CAG_59,
Coprobacillus_cateniformis, Enterococcus_casseliflavus, Clostridium_innocuum, Klebsiella_quasipneumoniae,
Clostridium_bolteae, Terrisporobacter_othiniensis, Parabacteroides_goldsteinii, Eubacterium_limosum,
Hungatella_hathewayi, Clostridium_aldenense, Megasphaera_micronuciformis, Acidaminococcus_fermentans,
Bacteroides_sp_CAG_462, Parabacteroides_sp_CAG_409, Prevotella_sp_CAG_1185, Desulfovibrio_piger,
Bacteroides_coprophilus, Eubacterium_sp_CAG_274, Brachyspira_sp_CAG_700, Flavonifractor_plautii,
Megamonas_hypermegale
SO4ASSIM-PWY: assimilatory sulfate reduction I
Citrobacter_freundii, Citrobacter_youngae, Enterobacter_cloacae_complex, Escherichia_coli,
Klebsiella_variicola, Citrobacter_braakii, Hafnia_paralvei, Klebsiella_oxytoca, Proteus_vulgaris,
Enterobacter_mori, Escherichia_fergusonii, Klebsiella_pneumoniae, Klebsiella_quasipneumoniae,
Proteus_hauseri, Proteus_penneri, Cronobacter_sakazakii, Haemophilus_parainfluenzae, Citrobacter_europaeus,
Citrobacter_farmeri, Citrobacter_werkmanii, Kluyvera_cryocrescens, Saccharomyces_cerevisiae,
Kluyvera_ascorbata, Raoultella_planticola, Klebsiella_michiganensis, Odoribacter_laneus,
Citrobacter_portucalensis
SULFATE-CYS-PWY: superpathway of sulfate assimilation and cysteine biosynthesis
Citrobacter_freundii, Escherichia_coli, Citrobacter_braakii, Hafnia_paralvei, Proteus_vulgaris,
Enterobacter_mori, Escherichia_fergusonii, Klebsiella_pneumoniae, Klebsiella_quasipneumoniae,
Proteus_hauseri, Enterobacter_cloacae_complex, Proteus_penneri, Cronobacter_sakazakii,
Haemophilus_parainfluenzae, Citrobacter_europaeus, Citrobacter_farmeri, Citrobacter_youngae,
Klebsiella_oxytoca, Klebsiella_variicola, Kluyvera_cryocrescens, Citrobacter_werkmanii,
Klebsiella_michiganensis, Citrobacter_portucalensis
TCA: TCA cycle I (prokaryotic)
Citrobacter_freundii, Enterobacter_cloacae_complex, Escherichia_coli, Citrobacter_braakii, Hafnia_paralvei,
Escherichia_fergusonii, Klebsiella_pneumoniae, Klebsiella_quasipneumoniae, Proteus_penneri,
Cronobacter_sakazakii, Citrobacter_europaeus, Citrobacter_farmeri, Citrobacter_werkmanii,
Citrobacter_youngae, Klebsiella_oxytoca, Klebsiella_variicola, Kluyvera_cryocrescens,
Saccharomyces_cerevisiae, Kluyvera_ascorbata, Klebsiella_michiganensis, Citrobacter_koseri,
Citrobacter_portucalensis
TEICHOICACID-PWY: poly(glycerol phosphate) wall teichoic acid biosynthesis
Bacteroides_vulgatus, Ruminococcus_torques, Parabacteroides_distasonis, Lactobacillus_plantarum,
Bacteroides_cellulosilyticus, Bacteroides_thetaiotaomicron, Bacteroides_uniformis
THISYRA-PWY: superpathway of thiamine diphosphate biosynthesis III (eukaryotes)
Bacteroides_fragilis, Bacteroides_ovatus, Bacteroides_uniformis, Ruminococcus_torques,
Faecalibacterium_prausnitzii, Eubacterium_rectale, Roseburia_intestinalis, Bacteroides_xylanisolvens,
Roseburia_faecis, Roseburia_hominis, Bifidobacterium_bifidum, Ruminococcus_gnavus, Dorea_longicatena,
Gordonibacter_pamelaeae, Lactobacillus_rhamnosus, Eubacterium_sulci, Holdemanella_biformis,
Mitsuokella_multacida, Butyrivibrio_crossotus, Catenibacterium_mitsuokai,
Phascolarctobacterium_succinatutens, Fusobacterium_mortiferum, Clostridium_innocuum,
Clostridium_aldenense, Bifidobacterium_angulatum, Clostridium_saccharolyticum, Clostridium_scindens
Pathway - IBS-U (Pathway: microbe)
ARGININE-SYN4-PWY: L-ornithine biosynthesis II
Bacteroides_cellulosilyticus, Bacteroides_dorei, Bacteroides_fragilis, Bacteroides_ovatus,
Bacteroides_thetaiotaomicron, Bacteroides_uniformis, Bacteroides_vulgatus, Bacteroides_xylanisolvens,
Bacteroides_massiliensis, Klebsiella_pneumoniae, Bacteroides_coprocola, Bacteroides_faecis,
Bacteroides_stercoris, Bacteroides_finegoldii, Paraprevotella_xylaniphila, Bacteroides_caccae,
Bacteroides_coprophilus, Bacteroides_nordii, Bacteroides_plebeius, Paraprevotella_clara,
Bacteroides_intestinalis, Bacteroides_faecis_CAG_32, Bacteroides_eggerthii, Bacteroides_clarus,
Catenibacterium_mitsuokai, Bacteroides_salyersiae
ECASYN-PWY: enterobacterial common antigen biosynthesis
Escherichia_coli, Klebsiella_pneumoniae, Citrobacter_braakii, Enterobacter_cloacae_complex,
Klebsiella_variicola, Kluyvera_ascorbata
GLUCARDEG-PWY: D-glucarate degradation I
Escherichia_coli, Klebsiella_pneumoniae, Citrobacter_braakii, Klebsiella_oxytoca, Clostridium_clostridioforme,
Enterobacter_bugandensis, Enterobacter_cloacae_complex, Klebsiella_variicola, Kluyvera_ascorbata
GLUCUROCAT-PWY: superpathway of &beta;-D-glucuronosides degradation
Bacteroides_fragilis, Escherichia_coli, Faecalibacterium_prausnitzii, Citrobacter_braakii,
Intestinimonas_butyriciproducens, Kluyvera_ascorbata, Lactobacillus_rhamnosus
GLYCOLYSIS: glycolysis I (from glucose 6-phosphate)
Bacteroides_cellulosilyticus, Bacteroides_fragilis, Escherichia_coli, Klebsiella_pneumoniae, Citrobacter_braakii,
Klebsiella_michiganensis, Klebsiella_oxytoca, Bacteroides_nordii, Hafnia_alvei, Enterobacter_cloacae_complex,
Catenibacterium_mitsuokai, Klebsiella_variicola, Kluyvera_ascorbata, Parabacteroides_distasonis,
Bacteroides_salyersiae, Lactobacillus_rhamnosus
GOLPDLCAT-PWY: superpathway of glycerol degradation to 1,3-propanediol
Ruminococcus_torques, Flavonifractor_plautii, Escherichia_coli, Klebsiella_pneumoniae, Klebsiella_variicola,
Anaerostipes_hadrus, Eubacterium_hallii, Citrobacter_braakii, Citrobacter_youngae, Klebsiella_oxytoca,
Enterobacter_cloacae_complex, Klebsiella_quasipneumoniae
HISTSYN-PWY: L-histidine biosynthesis
Alistipes_finegoldii, Alistipes_shahii, Bacteroides_cellulosilyticus, Bacteroides_dorei, Bacteroides_fragilis,
Bacteroides_ovatus, Bacteroides_thetaiotaomicron, Bacteroides_uniformis, Bacteroides_vulgatus,
Bacteroides_xylanisolvens, Barnesiella_intestinihominis, Bifidobacterium_bifidum, Bifidobacterium_longum,
Blautia_obeum, Ruminococcus_torques, Collinsella_aerofaciens, Escherichia_coli, Faecalibacterium_prausnitzii,
Flavonifractor_plautii, Parabacteroides_merdae, Roseburia_intestinalis, Ruminococcus_bromii,
Bacteroides_caccae, Bacteroides_massiliensis, Blautia_hydrogenotrophica, Eggerthella_lenta,
Clostridium_innocuum, Klebsiella_pneumoniae, Klebsiella_quasipneumoniae, Parabacteroides_distasonis,
Bacteroides_coprocola, Bacteroides_faecis, Bacteroides_stercoris, Lactococcus_lactis, Megamonas_funiformis,
Roseburia_inulinivorans, Clostridium_sp_CAG_964, Agathobaculum_butyriciproducens,
Anaerotignum_lactatifermentans, Parabacteroides_johnsonii, Streptococcus_salivarius, Bacteroides_finegoldii,
Eubacterium_siraeum, Bacteroides_intestinalis, Bacteroides_salyersiae, Erysipelatoclostridium_ramosum,
Paraprevotella_xylaniphila, Bifidobacterium_adolescentis, Citrobacter_braakii, Dialister_sp_CAG_357,
Eubacterium_sp_CAG_180, Eubacterium_sp_CAG_251, Klebsiella_michiganensis, Klebsiella_oxytoca,
Proteobacteria_bacterium_CAG_139, Hungatella_hathewayi, Parabacteroides_goldsteinii,
Dialister_succinatiphilus, Bacteroides_nordii, Bacteroides_plebeius, Paraprevotella_clara,
Prevotella_sp_CAG_279, Bacteroides_faecis_CAG_32, Bifidobacterium_pseudocatenulatum,
Catenibacterium_mitsuokai, Eubacterium_sp_CAG_38, Prevotella_copri, Prevotella_sp_AM42_24,
Prevotella_sp_CAG_5226, Roseburia_hominis, Eisenbergiella_massiliensis, Lawsonibacter_asaccharolyticus,
Parasutterella_excrementihominis, Bacteroides_clarus, Firmicutes_bacterium_CAG_94, Anaerostipes_caccae,
Blautia_producta, Clostridium_clostridioforme, Clostridium_scindens, Veillonella_seminalis,
Dorea_formicigenerans, Hafnia_alvei, Acinetobacter_johnsonii, Acinetobacter_ursingii,
Enterobacter_bugandensis, Enterobacter_cloacae_complex, Pseudomonas_aeruginosa_group,
Streptococcus_thermophilus, Bifidobacterium_catenulatum, Veillonella_parvula, Anaerotruncus_sp_CAG_528,
Megasphaera_sp_DISK_18, Odoribacter_splanchnicus, Klebsiella_variicola, Alistipes_onderdonkii,
Bacteroides_sp_CAG_144, Lactobacillus_rhamnosus, Clostridium_leptum, Parabacteroides_sp_CAG_409
KETOGLUCONMET-PWY: ketogluconate metabolism
Escherichia_coli, Klebsiella_pneumoniae, Enterobacter_cloacae_complex, Citrobacter_braakii,
Klebsiella_oxytoca, Pseudomonas_aeruginosa_group, Pseudomonas_lundensis, Klebsiella_variicola,
Enterococcus_faecium
METH-ACETATE-PWY: methanogenesis from acetate
Blautia_obeum, Blautia_wexlerae, Ruminococcus_torques, Fusicatenibacter_saccharivorans,
Blautia_hydrogenotrophica, Blautia_sp_CAG_257
GLIPASYN-PWY: lipid IVA biosynthesis (E. coli)
Bacteroides_cellulosilyticus, Bacteroides_uniformis, Escherichia_coli, Klebsiella_pneumoniae,
Bacteroides_stercoris, Citrobacter_braakii, Klebsiella_michiganensis, Klebsiella_oxytoca,
Proteobacteria_bacterium_CAG_139, Bacteroides_intestinalis, Parasutterella_excrementihominis, Hafnia_alvei,
Pseudomonas_fragi, Acinetobacter_johnsonii, Acinetobacter_ursingii, Enterobacter_bugandensis,
Enterobacter_cloacae_complex, Pseudomonas_aeruginosa_group, Haemophilus_parainfluenzae,
Catenibacterium_mitsuokai, Klebsiella_variicola, Kluyvera_ascorbata
P124-PWY: Bifidobacterium shunt
Bifidobacterium_longum, Bifidobacterium_pseudocatenulatum, Collinsella_aerofaciens
P161-PWY: acetylene degradation (anaerobic) Escherichia_coli, Flavonifractor_plautii,
Klebsiella_pneumoniae, Enterobacter_cloacae_complex, Klebsiella_michiganensis, Lactococcus_lactis,
Citrobacter_braakii, Citrobacter_youngae, Klebsiella_oxytoca, Bifidobacterium_animalis, Hafnia_alvei,
Enterobacter_mori, Klebsiella_variicola, Kluyvera_ascorbata
P221-PWY: octane oxidation
Escherichia_coli, Klebsiella_pneumoniae, Klebsiella_quasipneumoniae, Hafnia_alvei, Acinetobacter_johnsonii,
Enterobacter_cloacae_complex, Pseudomonas_aeruginosa_group, Kluyvera_ascorbata
PENTOSE-P-PWY: pentose phosphate pathway Bacteroides_cellulosilyticus,
Bacteroides_xylanisolvens, Bifidobacterium_bifidum, Escherichia_coli, Klebsiella_pneumoniae,
Parabacteroides_distasonis, Bacteroides_fragilis, Citrobacter_braakii, Enterococcus_faecalis, Klebsiella_oxytoca,
Bacteroides_thetaiotaomicron, Clostridium_clostridioforme, Enterobacter_bugandensis,
Enterobacter_cloacae_complex, Enterobacter_mori, Haemophilus_parainfluenzae,
Haemophilus_sp_HMSC71H05, Klebsiella_variicola, Enterococcus_faecium, Lactobacillus_rhamnosus
PHOSLIPSYN-PWY: superpathway of phospholipid biosynthesis I (bacteria)
Escherichia_coli, Fusicatenibacter_saccharivorans, Ruminococcus_gnavus, Klebsiella_pneumoniae,
Citrobacter_braakii, Klebsiella_michiganensis, Klebsiella_oxytoca, Hafnia_alvei, Acinetobacter_johnsonii,
Enterobacter_cloacae_complex, Pseudomonas_aeruginosa_group, Pseudomonas_lundensis,
Haemophilus_parainfluenzae, Haemophilus_sp_HMSC71H05, Klebsiella_variicola, Kluyvera_ascorbata,
Clostridium_leptum
POLYISOPRENSYN-PWY: polyisoprenoid biosynthesis (E. coli)
Escherichia_coli, Klebsiella_pneumoniae, Citrobacter_braakii, Klebsiella_oxytoca,
Enterobacter_cloacae_complex, Pseudomonas_lundensis, Klebsiella_variicola
PWY-4321: L-glutamate degradation IV
Escherichia_coli, Hafnia_alvei, Klebsiella_oxytoca
PWY-5030: L-histidine degradation III
Alistipes_finegoldii, Alistipes_shahii, Bacteroides_dorei, Bacteroides_fragilis, Bacteroides_ovatus,
Bacteroides_thetaiotaomicron, Bacteroides_uniformis, Bacteroides_vulgatus, Bacteroides_xylanisolvens,
Bacteroides_massiliensis, Streptococcus_parasanguinis, Alistipes_putredinis, Bacteroides_stercoris,
Alistipes_onderdonkii, Bacteroides_sp_OM05_12, Bacteroides_faecis, Bacteroides_clarus,
Catenibacterium_mitsuokai
PWY-5083: D(P)/DPH interconversion
Escherichia_coli, Klebsiella_pneumoniae, Citrobacter_braakii, Klebsiella_oxytoca,
Enterobacter_cloacae_complex, Enterobacter_mori, Klebsiella_quasipneumoniae, Klebsiella_variicola,
Kluyvera_ascorbata, Klebsiella_aerogenes
PWY-5104: L-isoleucine biosynthesis IV
Escherichia_coli, Parabacteroides_merdae, Eggerthella_lenta, Parabacteroides_distasonis, Citrobacter_braakii,
Parabacteroides_goldsteinii, Enterobacter_cloacae_complex, Klebsiella_oxytoca,
Pseudomonas_aeruginosa_group, Kluyvera_ascorbata
PWY-5189: tetrapyrrole biosynthesis II (from glycine)
Escherichia_coli, Klebsiella_pneumoniae, Klebsiella_michiganensis, Actinomyces_odontolyticus,
Actinomyces_sp_ICM47, Pseudomonas_fragi, Enterobacter_cloacae_complex, Klebsiella_oxytoca,
Pseudomonas_aeruginosa_group, Pseudomonas_lundensis, Stenotrophomonas_maltophilia
PWY-5345: superpathway of L-methionine biosynthesis (by sulfhydrylation)
PWY-5484: glycolysis II (from fructose 6-phosphate)
Bacteroides_cellulosilyticus, Bacteroides_fragilis, Escherichia_coli, Klebsiella_pneumoniae,
Klebsiella_variicola, Citrobacter_braakii, Klebsiella_michiganensis, Klebsiella_oxytoca, Bacteroides_nordii,
Hafnia_alvei, Enterobacter_cloacae_complex, Catenibacterium_mitsuokai, Kluyvera_ascorbata,
Parabacteroides_distasonis, Bacteroides_salyersiae, Lactobacillus_rhamnosus
PWY-5675: nitrate reduction V (assimilatory)
Escherichia_coli, Klebsiella_oxytoca, Enterobacter_bugandensis, Enterobacter_cloacae_complex,
Klebsiella_pneumoniae, Klebsiella_variicola, Kluyvera_ascorbata, Klebsiella_aerogenes
PWY-5695: inosine 5′-phosphate degradation
Alistipes_finegoldii, Alistipes_shahii, Anaerostipes_hadrus, Bacteroides_cellulosilyticus, Bacteroides_dorei,
Bacteroides_fragilis, Bacteroides_ovatus, Bacteroides_thetaiotaomicron, Bacteroides_uniformis,
Bacteroides_vulgatus, Bacteroides_xylanisolvens, Barnesiella_intestinihominis, Blautia_obeum,
Blautia_wexlerae, Ruminococcus_torques, Escherichia_coli, Faecalibacterium_prausnitzii,
Fusicatenibacter_saccharivorans, Clostridium_clostridioforme, Roseburia_faecis, Roseburia_intestinalis,
Bacteroides_massiliensis, Blautia_hydrogenotrophica, Coprococcus_comes, Dialister_invisus,
Dorea_longicatena, Klebsiella_pneumoniae, Lachnospira_pectinoschiza, Parabacteroides_distasonis,
Bacteroides_caccae, Bacteroides_coprocola, Bacteroides_stercoris, Eubacterium_eligens,
Roseburia_inulinivorans, Streptococcus_salivarius, Agathobaculum_butyriciproducens, Veillonella_atypica,
Alistipes_putredinis, Bacteroides_finegoldii, Alistipes_onderdonkii, Intestinimonas_butyriciproducens,
Parabacteroides_johnsonii, Bacteroides_galacturonicus, Citrobacter_braakii, Eubacterium_ramulus,
Eubacterium_ventriosum, Klebsiella_oxytoca, Lactobacillus_rogosae, Eisenbergiella_tayi,
Hungatella_hathewayi, Clostridium_symbiosum, Ruminococcus_bicirculans, Bacteroides_coprophilus,
Bacteroides_nordii, Bacteroides_plebeius, Parabacteroides_goldsteinii, Bacteroides_intestinalis,
Bacteroides_sp_OM05_12, Coprococcus_eutactus, Prevotella_sp_CAG_279, Bilophila_wadsworthia,
Flavonifractor_plautii, Clostridium_bolteae, Bacteroides_faecis, Bacteroides_faecis_CAG_32,
Catenibacterium_mitsuokai, Megasphaera_elsdenii, Prevotella_copri, Prevotella_sp_AM42_24,
Desulfovibrio_piger, Eisenbergiella_massiliensis, Odoribacter_splanchnicus, Bacteroides_clarus,
Firmicutes_bacterium_CAG_94, Veillonella_seminalis, Hafnia_alvei, Prevotella_sp_CAG_1031,
Acinetobacter_johnsonii, Acinetobacter_ursingii, Enterobacter_bugandensis, Enterobacter_cloacae_complex,
Enterobacter_mori, Pseudomonas_aeruginosa_group, Pseudomonas_lundensis, Klebsiella_variicola,
Bacteroides_sp_CAG_927, Megasphaera_sp_DISK_18, Veillonella_parvula, Bacteroides_eggerthii,
Kluyvera_ascorbata, Alistipes_indistinctus, Clostridium_citroniae, Bacteroides_salyersiae,
Enterococcus_faecalis, Enterococcus_faecium, Lactobacillus_rhamnosus, Alistipes_inops
PWY-5723: Rubisco shunt
Escherichia_coli, Klebsiella_pneumoniae, Citrobacter_braakii, Klebsiella_michiganensis,
Enterobacter_cloacae_complex, Klebsiella_variicola
PWY-5918: superpathway of heme b biosynthesis from glutamate
Escherichia_coli, Klebsiella_pneumoniae, Citrobacter_braakii, Klebsiella_michiganensis,
Actinomyces_odontolyticus, Pseudomonas_fragi, Enterobacter_cloacae_complex, Klebsiella_oxytoca,
Pseudomonas_aeruginosa_group, Klebsiella_variicola
PWY-5971: palmitate biosynthesis (type II fatty acid synthase)
Escherichia_coli, Citrobacter_braakii, Enterobacter_cloacae_complex
PWY-622: starch biosynthesis
PWY-6284: superpathway of unsaturated fatty acids biosynthesis (E. coli)
Escherichia_coli, Citrobacter_braakii, Enterobacter_bugandensis, Enterobacter_cloacae_complex
PWY-6507: 4-deoxy-L-threo-hex-4-enopyranuronate degradation
Escherichia_coli, Faecalibacterium_prausnitzii, Clostridium_aldenense, Citrobacter_braakii,
Hungatella_hathewayi, Clostridium_clostridioforme, Clostridium_bolteae, Hafnia_alvei,
Enterobacter_cloacae_complex, Kluyvera_ascorbata, Dorea_longicatena, Clostridium_citroniae,
Enterococcus_faecalis, Enterococcus_faecium
PWY-6545: pyrimidine deoxyribonucleotides de novo biosynthesis III
Escherichia_coli, Klebsiella_pneumoniae, Enterobacter_cloacae_complex, Klebsiella_variicola,
Kluyvera_ascorbata
PWY-6803: phosphatidylcholine acyl editing
Escherichia_coli, Klebsiella_pneumoniae, Citrobacter_braakii, Citrobacter_youngae, Klebsiella_oxytoca,
Citrobacter_portucalensis, Salmonella_enterica, Acinetobacter_johnsonii, Enterobacter_bugandensis,
Enterobacter_cloacae_complex, Stenotrophomonas_maltophilia, Klebsiella_variicola, Kluyvera_ascorbata
PWY-7184: pyrimidine deoxyribonucleotides de novo biosynthesis I
Escherichia_coli, Klebsiella_pneumoniae, Citrobacter_braakii, Klebsiella_michiganensis, Acinetobacter_ursingii,
Enterobacter_bugandensis, Enterobacter_cloacae_complex, Klebsiella_oxytoca, Haemophilus_parainfluenzae,
Klebsiella_variicola, Kluyvera_ascorbata
PWY-7204: pyridoxal 5′-phosphate salvage II (plants)
Escherichia_coli, Citrobacter_braakii, Klebsiella_michiganensis, Enterobacter_cloacae_complex
PWY-7210: pyrimidine deoxyribonucleotides biosynthesis from CTP
Escherichia_coli, Klebsiella_pneumoniae, Acinetobacter_ursingii, Enterobacter_bugandensis,
Enterobacter_cloacae_complex, Haemophilus_parainfluenzae, Klebsiella_variicola, Kluyvera_ascorbata
PWY-7211: superpathway of pyrimidine deoxyribonucleotides de novo biosynthesis
Escherichia_coli, Klebsiella_pneumoniae, Enterobacter_cloacae_complex, Klebsiella_variicola
PWY-7242: D-fructuronate degradation
Bacteroides_fragilis, Clostridium_sp_CAG_58, Escherichia_coli, Faecalibacterium_prausnitzii,
Lactococcus_lactis, Intestinimonas_butyriciproducens, Citrobacter_braakii, Citrobacter_youngae, Hafnia_alvei,
Klebsiella_oxytoca, Kluyvera_ascorbata, Enterococcus_faecalis, Enterococcus_faecium,
Lactobacillus_rhamnosus
PWY-7269: mitochondrial DPH production (yeast)
Escherichia_coli, Klebsiella_pneumoniae, Klebsiella_quasipneumoniae, Enterobacter_cloacae_complex,
Klebsiella_oxytoca, Pseudomonas_aeruginosa_group, Klebsiella_variicola, Kluyvera_ascorbata
PWY-7282: 4-amino-2-methyl-5-diphosphomethylpyrimidine biosynthesis II
Bacteroides_fragilis, Bacteroides_uniformis, Escherichia_coli, Bacteroides_xylanisolvens,
Klebsiella_pneumoniae, Bacteroides_ovatus, Streptococcus_salivarius, Citrobacter_braakii, Klebsiella_oxytoca,
Bacteroides_sp_OM05_12, Enterobacter_cloacae_complex, Pseudomonas_aeruginosa_group,
Pseudomonas_lundensis, Haemophilus_parainfluenzae, Haemophilus_sp_HMSC71H05,
Catenibacterium_mitsuokai, Klebsiella_variicola, Kluyvera_ascorbata
PWY-7392: taxadiene biosynthesis (engineered)
Klebsiella_pneumoniae, Enterobacter_cloacae_complex
PWY-7446: sulfoquinovose degradation I
Escherichia_coli, Citrobacter_braakii, Klebsiella_oxytoca, Enterobacter_bugandensis,
Enterobacter_cloacae_complex
PWY-7456: &beta;-(1,4)-mannan degradation
Eubacterium_siraeum, Coprococcus_eutactus, Roseburia_faecis
PWY0-1298: superpathway of pyrimidine deoxyribonucleosides degradation
Escherichia_coli, Klebsiella_pneumoniae, Lactococcus_lactis, Citrobacter_braakii, Citrobacter_youngae,
Klebsiella_michiganensis, Klebsiella_oxytoca, Hungatella_hathewayi, Enterobacter_bugandensis,
Enterobacter_cloacae_complex, Klebsiella_variicola, Kluyvera_ascorbata, Enterococcus_faecalis,
Enterococcus_faecium
PWY3O-355: stearate biosynthesis III (fungi)
Bacteroides_vulgatus, Bacteroides_dorei, Bacteroides_uniformis, Enterobacter_cloacae_complex,
Pseudomonas_aeruginosa_group
PWY4FS-7: phosphatidylglycerol biosynthesis I (plastidic)
Escherichia_coli, Fusicatenibacter_saccharivorans, Roseburia_intestinalis, Ruminococcus_gnavus,
Klebsiella_pneumoniae, Streptococcus_salivarius, Citrobacter_braakii, Klebsiella_michiganensis,
Klebsiella_oxytoca, Hafnia_alvei, Acinetobacter_johnsonii, Enterobacter_cloacae_complex,
Pseudomonas_aeruginosa_group, Stenotrophomonas_maltophilia, Haemophilus_parainfluenzae,
Haemophilus_sp_HMSC71H05, Streptococcus_thermophilus, Faecalibacterium_prausnitzii,
Catenibacterium_mitsuokai, Klebsiella_variicola, Kluyvera_ascorbata, Clostridium_leptum
PWY4FS-8: phosphatidylglycerol biosynthesis II (non-plastidic) Escherichia_coli,
Fusicatenibacter_saccharivorans, Roseburia_intestinalis, Ruminococcus_gnavus, Klebsiella_pneumoniae,
Streptococcus_salivarius, Citrobacter_braakii, Klebsiella_michiganensis, Klebsiella_oxytoca, Hafnia_alvei,
Acinetobacter_johnsonii, Enterobacter_cloacae_complex, Pseudomonas_aeruginosa_group,
Stenotrophomonas_maltophilia, Haemophilus_parainfluenzae, Haemophilus_sp_HMSC71H05,
Streptococcus_thermophilus, Faecalibacterium_prausnitzii, Catenibacterium_mitsuokai, Klebsiella_variicola,
Kluyvera_ascorbata, Clostridium_leptum
PWY4LZ-257: superpathway of fermentation (Chlamydomonas reinhardtii) Escherichia_coli,
Klebsiella_pneumoniae, Klebsiella_oxytoca, Hafnia_alvei, Enterobacter_cloacae_complex
PWY66-389: phytol degradation
Escherichia_coli, Klebsiella_pneumoniae, Klebsiella_quasipneumoniae,
Hafnia_alvei, Acinetobacter_johnsonii, Enterobacter_cloacae_complex, Pseudomonas_aeruginosa_group,
Kluyvera_ascorbata
PYRIDNUCSAL-PWY: D salvage pathway I (PNC VI cycle)
Escherichia_coli, Klebsiella_oxytoca, Enterobacter_cloacae_complex, Pseudomonas_aeruginosa_group,
Bifidobacterium_catenulatum
RIBOSYN2-PWY: flavin biosynthesis I (bacteria and plants)
Bacteroides_cellulosilyticus, Bacteroides_dorei, Bacteroides_fragilis, Bacteroides_ovatus,
Bacteroides_uniformis, Bacteroides_vulgatus, Bacteroides_xylanisolvens, Barnesiella_intestinihominis,
Blautia_obeum, Blautia_wexlerae, Ruminococcus_torques, Escherichia_coli, Faecalibacterium_prausnitzii,
Eubacterium_rectale, Parabacteroides_merdae, Phascolarctobacterium_faecium, Roseburia_faecis,
Roseburia_intestinalis, Bacteroides_massiliensis, Coprococcus_comes, Dorea_longicatena,
Clostridium_innocuum, Klebsiella_pneumoniae, Lachnospira_pectinoschiza, Parabacteroides_distasonis,
Anaerostipes_hadrus, Clostridium_sp_CAG_299, Dorea_formicigenerans, Eubacterium_eligens,
Lactococcus_lactis, Megamonas_funiformis, Akkermansia_muciniphila, Clostridium_sp_CAG_964,
Agathobaculum_butyriciproducens, Anaerotignum_lactatifermentans, Bacteroides_thetaiotaomicron,
Eubacterium_hallii, Parabacteroides_johnsonii, Phascolarctobacterium_sp_CAG_266, Bacteroides_caccae,
Bacteroides_stercoris, Paraprevotella_xylaniphila, Bacteroides_galacturonicus, Citrobacter_braakii,
Dialister_sp_CAG_357, Klebsiella_michiganensis, Lactobacillus_rogosae, Roseburia_hominis,
Roseburia_sp_CAG_303, Fusobacterium_ulcerans, Fusobacterium_varium, Hungatella_hathewayi,
Clostridium_clostridioforme, Veillonella_dispar, Veillonella_parvula, Dialister_succinatiphilus,
Megamonas_hypermegale, Paraprevotella_clara, Bacteroides_intestinalis, Bacteroides_sp_OM05_12,
Coprococcus_eutactus, Prevotella_sp_CAG_279, Bacteroides_finegoldii, Bacteroides_nordii,
Bilophila_wadsworthia, Flavonifractor_plautii, Clostridium_bolteae, Bacteroides_faecis, Butyrivibrio_crossotus,
Eubacterium_sp_CAG_38, Phascolarctobacterium_succinatutens, Prevotella_copri, Prevotella_sp_AM42_24,
Desulfovibrio_piger, Coprobacter_fastidiosus, Clostridium_sp_CAG_167, Odoribacter_splanchnicus,
Ruminococcus_callidus, Veillonella_atypica, Bacteroides_clarus, Anaerostipes_caccae, Clostridium_symbiosum,
Bacteroides_coprocola, Prevotella_sp_CAG_1031, Acinetobacter_johnsonii, Acinetobacter_ursingii,
Enterobacter_bugandensis, Enterobacter_cloacae_complex, Klebsiella_oxytoca,
Pseudomonas_aeruginosa_group, Pseudomonas_lundensis, Eubacterium_ramulus, Veillonella_infantium,
Acidaminococcus_intestini, Bacteroides_sp_CAG_927, Butyricicoccus_pullicaecorum,
Ruminococcaceae_bacterium_D16, Catenibacterium_mitsuokai, Klebsiella_variicola, Kluyvera_ascorbata,
Bacteroides_salyersiae, Adlercreutzia_equolifaciens, Asaccharobacter_celatus
SO4ASSIM-PWY: assimilatory sulfate reduction I
Escherichia_coli, Klebsiella_pneumoniae, Citrobacter_braakii, Klebsiella_michiganensis, Klebsiella_oxytoca,
Acinetobacter_johnsonii, Enterobacter_bugandensis, Enterobacter_cloacae_complex,
Pseudomonas_aeruginosa_group, Pseudomonas_lundensis, Klebsiella_variicola, Kluyvera_ascorbata
SULFATE-CYS-PWY: superpathway of sulfate assimilation and cysteine biosynthesis
Escherichia_coli, Klebsiella_pneumoniae, Citrobacter_braakii, Klebsiella_michiganensis, Klebsiella_oxytoca,
Acinetobacter_johnsonii, Enterobacter_bugandensis, Enterobacter_cloacae_complex, Klebsiella_variicola,
Kluyvera_ascorbata
TCA: TCA cycle I (prokaryotic)
Escherichia_coli, Klebsiella_pneumoniae, Citrobacter_braakii, Klebsiella_michiganensis, Klebsiella_oxytoca,
Acinetobacter_johnsonii, Acinetobacter_ursingii, Enterobacter_bugandensis, Enterobacter_cloacae_complex,
Klebsiella_variicola, Kluyvera_ascorbata
TEICHOICACID-PWY: poly(glycerol phosphate) wall teichoic acid biosynthesis
Ruminococcus_torques, Bacteroides_uniformis, Parabacteroides_distasonis, Bacteroides_vulgatus,
Bacteroides_thetaiotaomicron
THISYRA-PWY: superpathway of thiamine diphosphate biosynthesis III (eukaryotes)
Bacteroides_fragilis, Bacteroides_uniformis, Bacteroides_xylanisolvens, Bifidobacterium_bifidum,
Ruminococcus_torques, Faecalibacterium_prausnitzii, Eubacterium_rectale, Roseburia_faecis,
Roseburia_intestinalis, Clostridium_innocuum, Ruminococcus_gnavus, Clostridium_hiranonis,
Bacteroides_ovatus, Dorea_longicatena, Roseburia_hominis, Butyrivibrio_crossotus,
Phascolarctobacterium_succinatutens, Gordonibacter_pamelaeae, Clostridium_scindens,
Catenibacterium_mitsuokai, Eubacterium_hallii, Holdemanella_biformis

The enterobacterial common antigen (ECA) biosynthesis pathway was associated with IBS-A. The ECA is one of the components of the outer membrane of Gram-negative bacteria and its association with IBS-A may indicate the increased presence of Enterobacterales in the gut microbiome. Interestingly, the ECA may contribute to virulence and protection of enteric pathogens from bile salts and antibiotics. Bile acids have been shown to protect the host from infection which may contribute to overall gut intestinal health. ECA protection against bile acids and antibiotics may make IBS-A difficult to treat with antibiotics and may contribute to dysbiosis. These results suggest that common antibiotic treatments for IBS may not be ideal for alleviating symptoms or treating the possible underlying microbiome triggers associated with IBS-A.

Pathways associated with healthy microbiomes were amino acid and ribonucleotide synthesis, polysaccharide degradation, and fermentation. L-methionine biosynthesis by sulfhydrylation and cysteine biosynthesis implies the presence of hydrogen sulfide in the gut. An overabundance of hydrogen sulfide induces inflammation, while low levels protect the gut lining and microbes against reactive oxygen species. Polysaccharide degradation, specifically beta-mannan degradation, is primarily driven by Roseburia intestinalis and the metabolic output has been shown to promote gut health. As products of fermentation, the role of SCFA has been implicated in cardiovascular and neurologic pathologies. The thiamine diphosphate biosynthesis pathway was associated with healthy gut metagenomes while negatively associated with IBS-A (FIG. 2). A thiamine deficiency has been shown to increase risk for lifelong neurodevelopmental consequences and is associated with many cardiovascular diseases. These results demonstrate the balance of metabolites must be regulated to maintain gut homeostasis and overall health. When the chemical and microbiome balance is disrupted, host physiology may be affected, leading to worsening gut symptoms or onset of disease.

IBS is heterogeneous; a universal cocktail of probiotics may not comprehensively target all symptoms experienced by individuals with IBS. Therefore, one of our goals is to individually formulate prebiotics and probiotics to address the more common symptoms experienced by individuals with IBS. There were three common strains included in formulas to specifically target constipation and diarrhea. Bifidobacterium longum was included in formulations for constipation. B. breve and Lactobacillus rhamnosus were included in formulations for diarrhea. Each of these probiotics were added to formulas for a total of 4-8 different probiotic strains at different concentrations.

In probiotic studies, most strains are detectable for less than 2 weeks following the cessation of probiotic supplementation. Because individuals in this study took daily probiotic supplements across a longitudinal time period (4 months between each microbiome test), we investigated microbiome changes in response to daily probiotic supplementation. In the population, there was no significant change in alpha or beta diversity across time, but there may have been changes in diversity within the individual (FIG. 3). There was a shift in beta diversity of the microbiome from one timepoint to the next, indicating there may be changes in microbiome composition in response to probiotic supplementation (FIG. 3). Diversity metrics that compare population level information may not show the impact of probiotic usage, but may influence smaller communities within the gut with postbiotic release.

In addition to the diversity metrics calculated, an overall microbiome score was calculated to take into consideration the microbiome composition and health and diet survey. Across timepoints 1-3, there was a significant increase in the microbiome score, indicating an improvement in the overall microbiome and symptoms in response to precision probiotic supplementation. When investigating individual probiotics, Bifidobacterium longum did not significantly increase across timepoints in the IBS subjects even when provided in precision formulations. The presence of B. longum may promote gut health through cross-feeding mechanisms that lead to the production of short chain fatty acids. B. breve and L. rhamnosus increased in relative abundance across time in individuals with IBS, indicating colonization of the gut microbiome that may contribute to changes in microbiome physiology. Further investigation is needed to identify potential functional changes in microbiome metabolism with daily prebiotic and probiotic supplementation in IBS and whether symptoms associated with IBS has been improved.

There are several limitations to this current study. First, the self-reporting nature of IBS is a limitation to this study. For official diagnosis of IBS, the Rome IV criteria assesses symptoms related to stool consistency and appearance, recurrent abdominal pain, and changes in bowel habits. Although the health and diet questionnaire included questions regarding gut symptoms and chronic conditions, a formal diagnosis was not verified. Second, this study was not designed to investigate longitudinal assessments of comprehensive gut issues experienced by the individuals with IBS. This hindered the ability to identify whether gut symptoms were alleviated by daily probiotic supplementation or whether there were associations with certain probiotic formulations in improving certain symptoms in IBS. However, because the relative abundance of the common probiotics formulated for constipation and diarrhea were increased in relative abundance across time, these results may inform future studies. Additional research is also needed to determine the roles of specific pathways in disease etiology of IBS.

In summary, differentially abundant microbes and functional pathways associated with IBS are reported. These data may help inform future studies and therapeutic strategies by identifying important microbes and pathways associated with each of the different IBS subtypes. As IBS is a multi-factorial syndrome, there is no one-size-fits-all approach to target all symptoms experienced by individuals with IBS. For potential life-style modifications in addition to probiotic supplementation, diet changes may also be an important factor in alleviating symptoms or changing the microbiome. Low FODMAP diet (LFD) and low lactose diet (LLD) reduced the IBS-SSS score. IBS subjects on LFD had significantly less abdominal pain, bloating, and gas production. When diet changes are not sufficient in alleviating symptoms, precision probiotics may help specifically target individual needs, although further research is needed to identify the long-term implications of prebiotic and probiotic supplementation on health.

### Conclusion

Microbes and pathways that differentiate healthy and IBS microbiomes were identified. In response to precision probiotic supplementation, the inventors identified a significant improvement in the overall microbiome score in individuals with IBS. These results suggest an important role for probiotics in the management of IBS symptoms.

### Example 2

### Alterations in Gut Microbiome Composition and Functional Potential in ASD

Early development of the gut microbiota plays an essential role, if not required, for healthy Gastrointestinal (GI) function. In the last five years, studies have associated this GI function to our nervous system and functions through the Gut-Brain axis. The mechanisms of how these systems communicate with one another are still under intense exploration. The association and result of impaired neurodevelopmental function through the gut-brain axis has been reported in several studies. Recovery of that function by influencing the gut microbiota has also been demonstrated. Here we look to discuss further the mechanisms by which these two systems are connected in the onset or impact of autism spectrum disorder and the extent to which probiotics have been studied to be a plausible intervention.

Metagenomics (the analysis of DNA from all organism's within a sample), computational speeds, and software have rapidly advanced our ability to access the microflora of an individual's gut. There is great variability in the human gut microbiome (the community of organisms that make up a human gut microflora) across individuals with differences associated with host diet, which affect gut microbiome communities at the abundance and functional levels, intake of drugs and antibiotics, interactions with pathogens and parasites and lastly, interspecies interactions and engagement with their environment. It has been observed that the stratification of autism spectrum disorder (ASD) treatment generated by the current classification process may not be effective for the personalization of early treatments, and there is a need to identify biologically significant parameters (biomarkers) that have the power to characterize everyone at different stages of neurological development automatically. This new approach to ASD has led to landmark research showing the value of using the microbiome to assess potential human physiological impacts, and modulation of that microbiota in ASD has now been shown to improve the symptoms and severity of ASD with potentially lasting effects for years. This work's goal is to understand better the underlying mechanisms that could contribute to such success and provide more deliberate and precise solutions to modulate the microbiome, including fortified precision probiotic formulations.

Most studies to date, related to the microbiome, preferentially look at GI symptoms as these present in 30-50% of all cases of ASD. This lack of data stratification by typical ASD symptoms could be why clinical improvement has been only reported anecdotally in children with ASD who develop fever, receive oral antibiotics, or ingest probiotics. These studies typically lack the power in sample size, stratification of the data sets, and lack of resolution on the data sets when looking at the microbiome (16s vs. WGS).

Disclosed herein are methods for identifying microbial signatures from samples that determine discriminant features between healthy control populations and subjects with autism spectrum disorder. Metagenomic sequencing captures all genomic content of the sample, providing insight into microbial identity and protein coding and non-coding genetic materials. Microbes include eukaryotes and prokaryotes, including bacteria, parasites, archaea, fungi, viruses, phage, and others. Genomic content that infers functional potential was assessed to find the underlying differences in microbial metabolisms between the subject populations.

### Materials and Methods

### Participants and Sample Collection.

Participants of the study were required to submit paperwork for a clinical diagnosis of autism by a medical professional. In addition, participants should not have had antibiotics within the last two months and were advised against changing medication, nutritional supplements, therapies, or dietary habits for the duration of the study. Required surveys were assigned at the beginning of the study to collect baseline information about the participants regarding dietary and nutritional habits, birth and infancy history, allergies, social responsiveness scale (SRS2), screen for child anxiety related disorders (SCARED), gastrointestinal severity rating scale (GSRS), and parental global impressions of autism (PGIA). A total of 315 participants with autism and 123 healthy NT, age matched controls from the Sun Genomics customer base were included in this study (Table 4). Extensive survey data part of the autism study was not collected from the healthy NT control population.

TABLE 4
Study cohort demographics. Listed are the number of subjects, age, and gender of
each cohort and the summaries of surveys taken by ASD study participants. The PGIA
survey is the parental global impressions of autism. SCARED is the screen for childhood
related anxiety disorders. SRS2 is the social responsiveness scale. GSRS is the
gastrointestinal symptom response scale. The nutrition survey assesses dietary habits
and number of daily servings of a variety of food categories. Values within the
parentheses indicate SD from the mean. Percentages indicate the proportion of the
population that fall within a subcategory (SCARED) or are indicative a specific
condition or phenotype (SRS2 and nutritional assessment surveys).

| | | ASD | | NT |
|---|---|---|---|---|
| N | | 315 | | 123 |
| Age | | 10.41 | (7.14) | 10.74 (8.71) |
| % Male | | 79.7% | | 52.03% |
| Shannon Index | | 3.90 | (0.48)* | 4.04 (0.38)* |
| PGIA | Overall | 2.81 | (0.87) | — |
| SCARED | Overall | 19.34 (15.46), 26.11% | | — |
| | Panic Disorder | 19.34 (15.46), 17.83% | | — |
| | Generalized Anxiety Disorder | 4.04 (4.44), 19.11% | | — |
| | Separation Anxiety | 4.42 (3.74), 42.04% | | — |
| | Social Anxiety Disorder | 5.49 (4.12), 29.30% | | — |
| | Significant School Avoidance | 1.52 (1.87), 27.39% | | — |
| SRS2 | Overall | 80.08 | (10.36) | — |
| | Mild to Moderate | 6.67% | | — |
| | Moderate | 29.17% | | — |
| | Severe | 64.17% | | — |
| GSRS | Overall | 2.25 | (0.97) | — |
| | Abdominal pain | 3.37 | (1.26) | — |
| | Constipation | 2.69 | (1.61) | — |
| | Diarrhea | 2.01 | (1.22) | — |
| | Ingestion Syndrome | 2.39 | (1.8) | — |
| | Reflux Syndrome | 1.69 | (1.16) | — |
| Nutrition | Overall | 21.07 | (14.71) | — |

| | | |
|---|---|---|
| Excellent/Very Good | 17.5% | — |
| Average | 22.6% | — |
| Below Average/Poor | 59.8% | — |

Stool samples were collected via the Flore™ research edition kit for metagenomic sequencing. The stool sample was collected by the parent or participant of the study with provided kit instructions. Stool samples collected from the control population were collected with the gut microbiome test kit (Flore™ gut health test kit). Samples were mailed to the Flore™ lab for analysis.

### Survey.

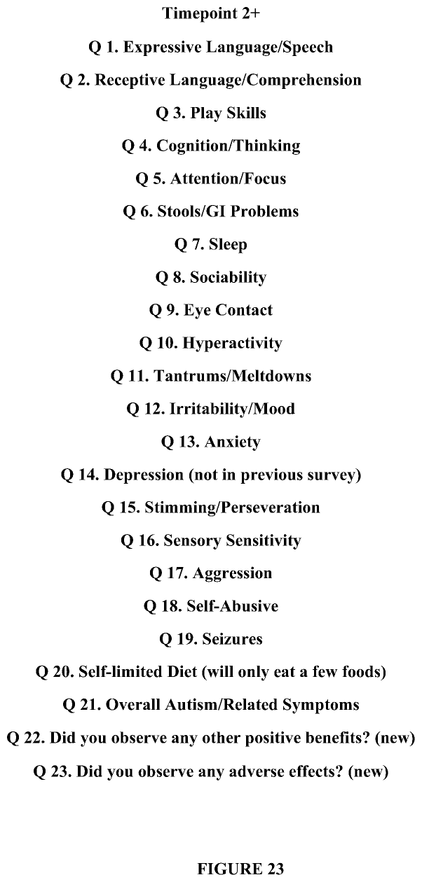

Participants were surveyed at T1, T2 and later time points using the survey questions set forth in FIGS. 22 and 23. A survey summary is shown in Table 4.

### Metagenomic Sequencing and Bioinformatic Analysis.

DNA extraction and library preparation methods are previously described in Phan et al. 2021. Briefly, DNA was extracted and purified with a proprietary method (U.S. Pat. Nos. 10,428,370 and 10,837,046, incorporated herein by reference in their entireties). Library prep and size selection was performed with NEBNext™ reagents and MagJet™ magnetic beads, respectively. Library quantitation was performed with qPCR prior to normalization. Libraries were sequenced on an Illumina NextSeq™ 550 (Illumina, San Diego, CA). Once sequenced, reads were quality filtered and processed to remove human reads. For taxonomic analysis, reads were aligned to a hand-curated database of 23,000 species. Metabolic pathways and gene families were determined using HUMAnN2™.

### Statistical Analyses.

Downstream statistical analyses were performed in R. Permutational multivariate analysis of variance (PERMANOVA) was performed with the “adonis” function from the “vegan” package. Bray-Curtis dissimilarity was calculated with the “vegan” package and was used to for principal-coordinates analysis. Pearson correlations were used to investigate parametric relationships between variables of interest. Random forest was performed to identify variables of importance between the two cohorts in the microbiome, pathways, and gene family datasets. The random forest analysis was performed with the randomForest package in R, but may also be performed with other packages, including caret in R or scikit-learn in Python. Subsequent significance testing with FDR corrections was performed on the random forest variables of importance to compare relative abundances of microbes from each cohort.

Statistical analyses include univariate, multivariate, and machine learning approaches to profile community wide microbial features. Permutational multivariate analysis of variance calculates the percentage of variation explained by disease status. For example, in Table 5, 2.5% of the variation in the microbiome composition data significantly distinguishes healthy and ASD. Principal coordinates analysis displays the differences in the microbiome compositions between the healthy and ASD populations. To find associations between the microbial community and functional potential, correlations between the microbes and genetic pathways were investigated. A random forest model generated based on the abundances of each member of the microbial community and genetic pathways distinguished features between the healthy and ASD cohorts. The relative abundances of the microbial species from the model was then plotted against the principal coordinate to visualize differences in relative abundances of microbes across the two populations.

TABLE 5
Permutational multivariate analysis of variance of microbiome
composition. Multivariate analysis method to determine the percent
variation of the data that are attributed to sample features
(Healthy vs. ASD). Approximately 2.5% of the variation explained
in the data distinguish the status of healthy and ASD.

| | R² | P value |
|---|---|---|
| Status (Healthy vs. ASD) | 0.025 | 0.001 |
| Residuals | 0.975 | |

### Results

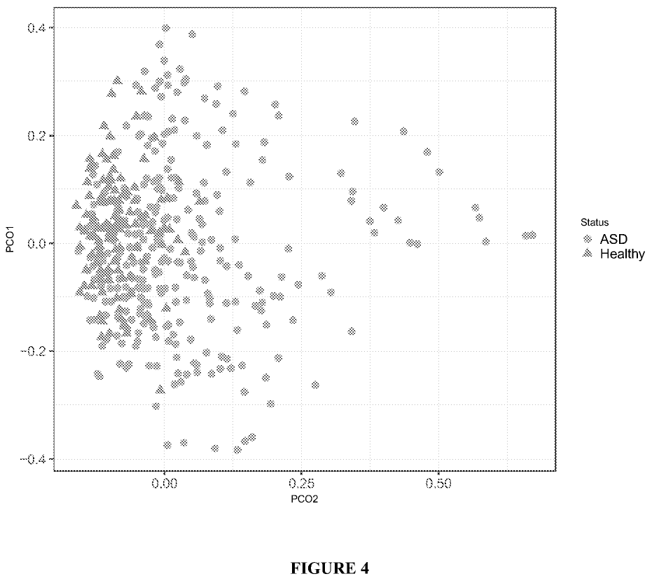

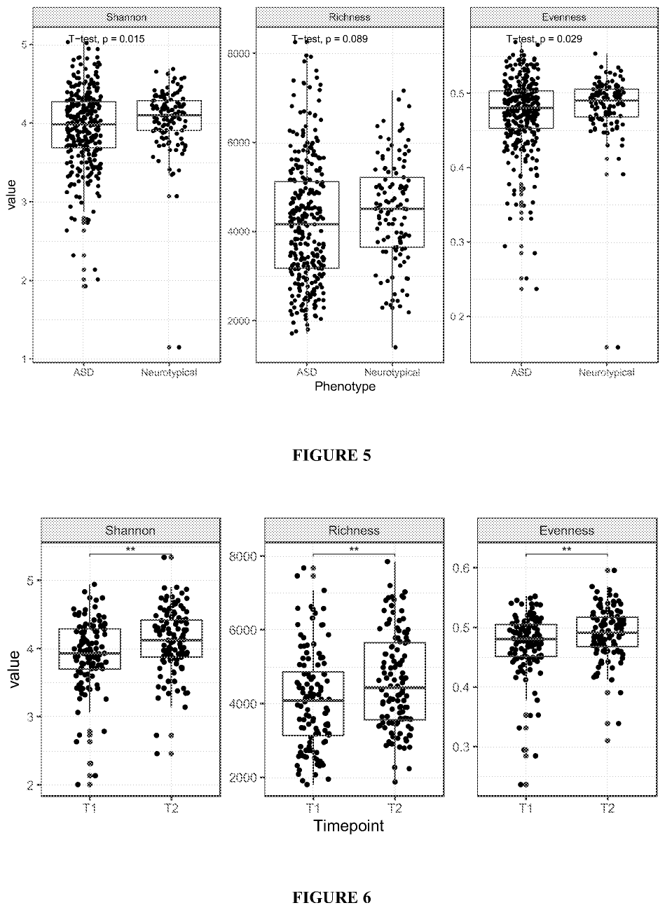

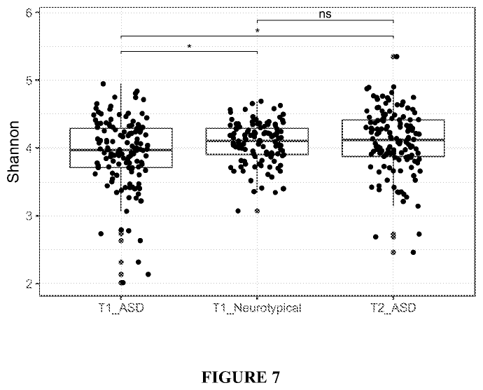

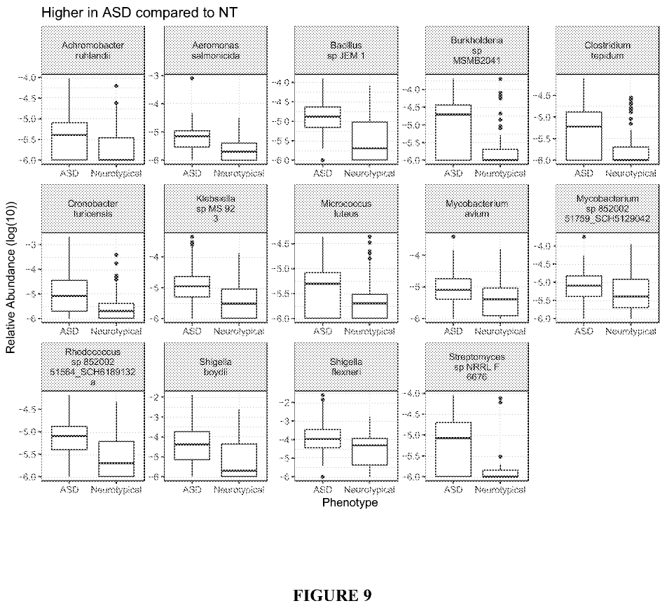

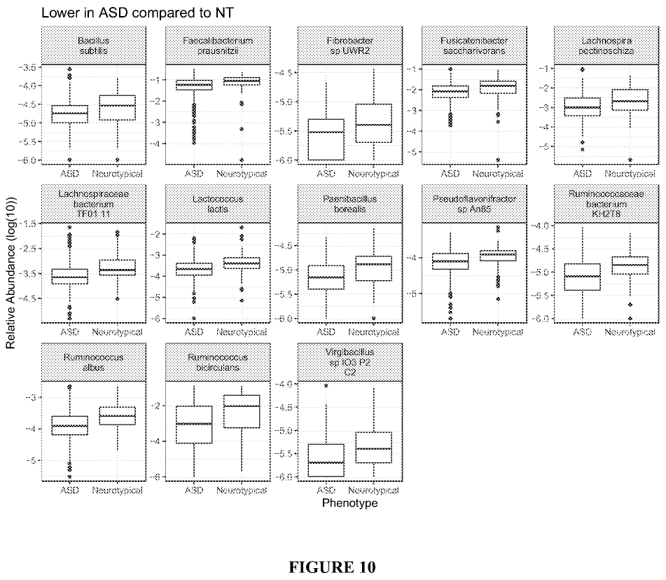

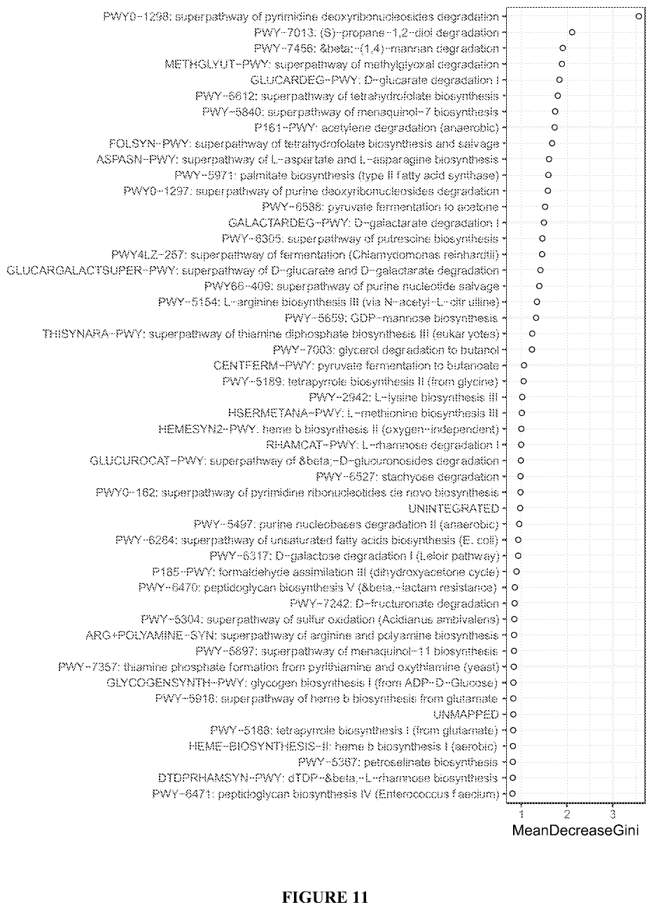

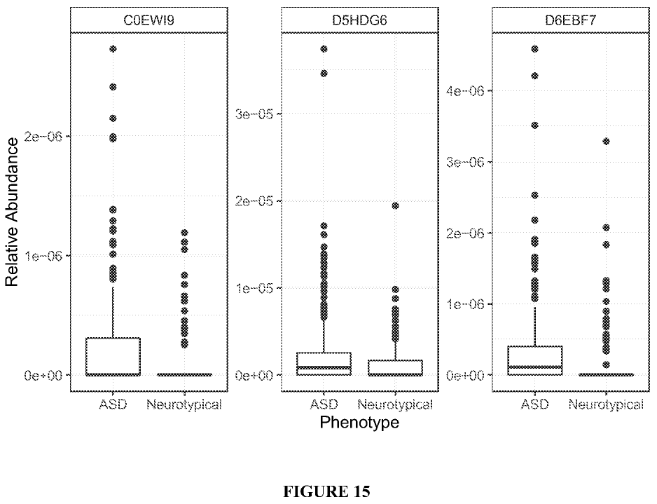

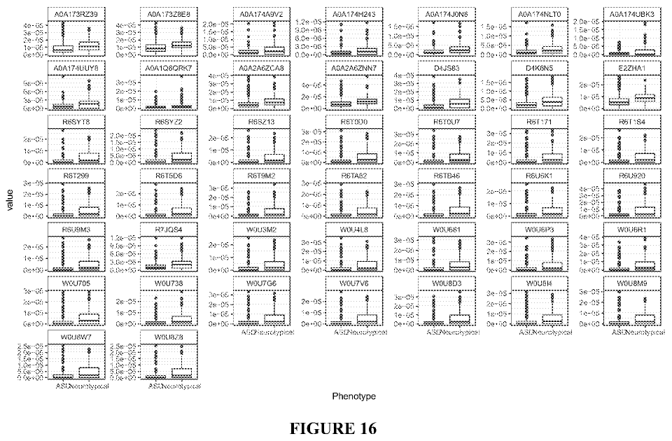

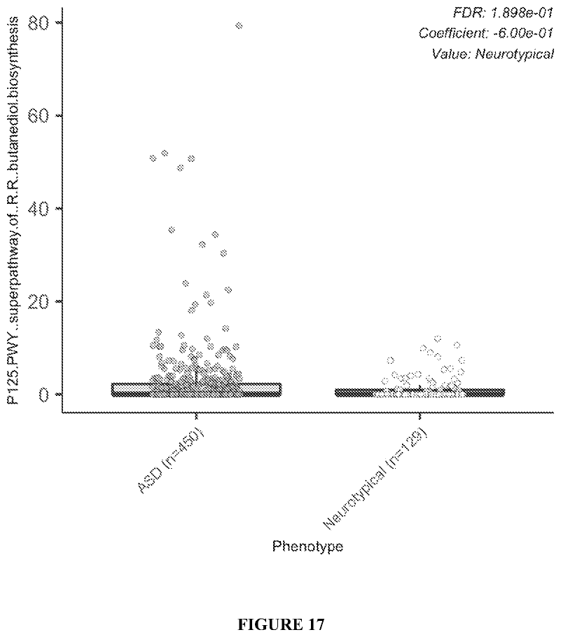

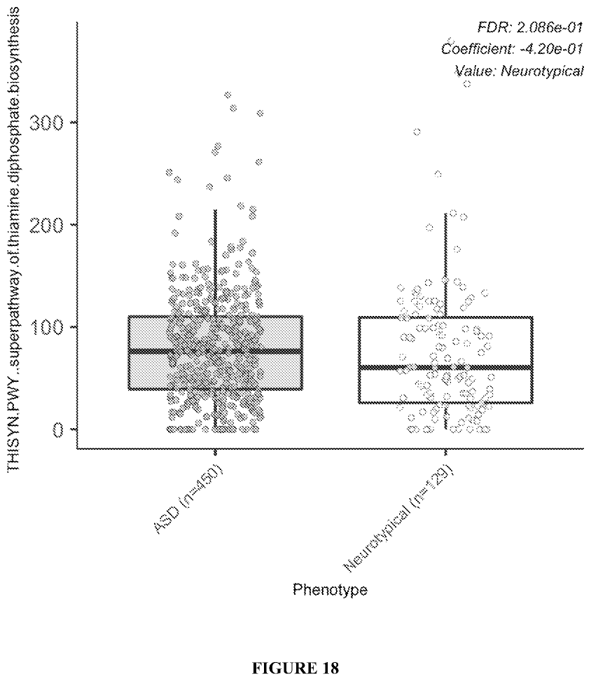

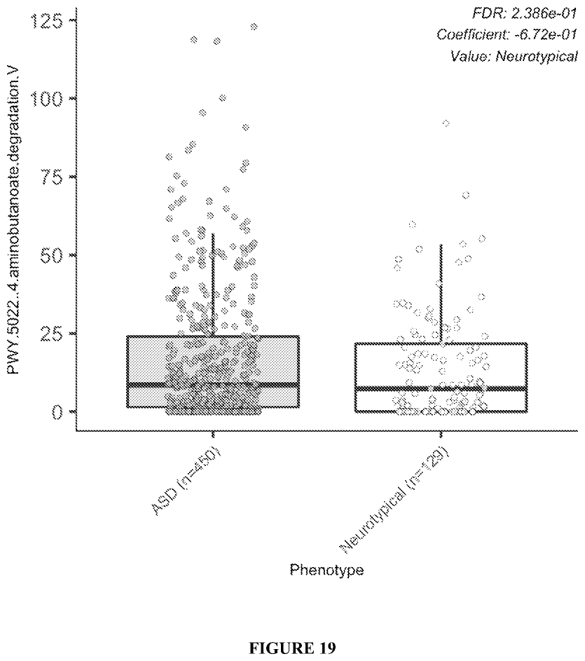

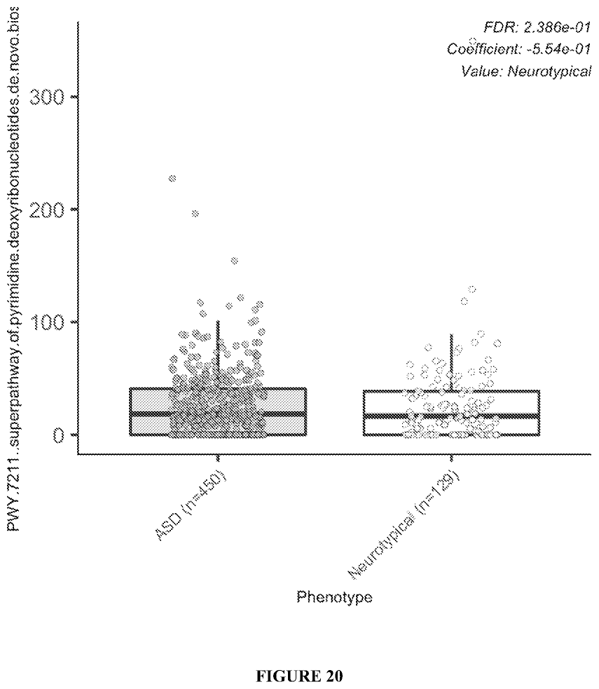

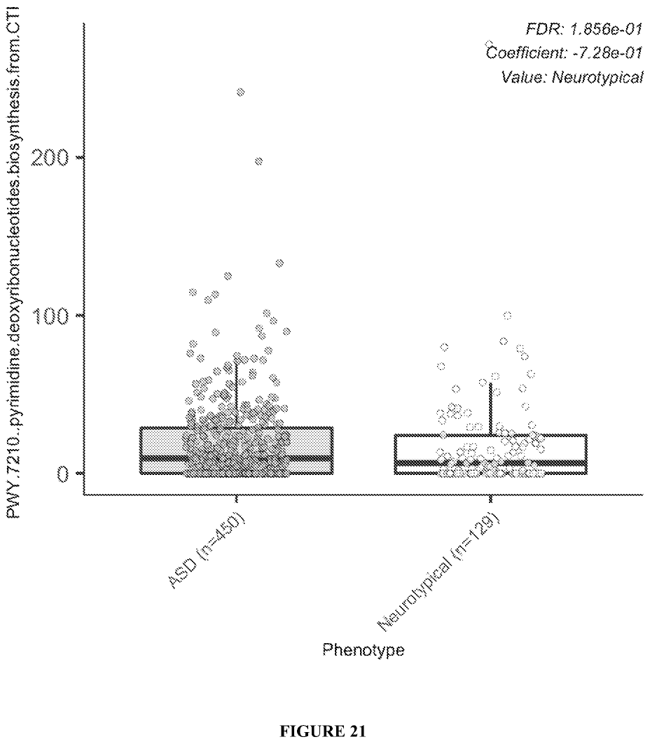

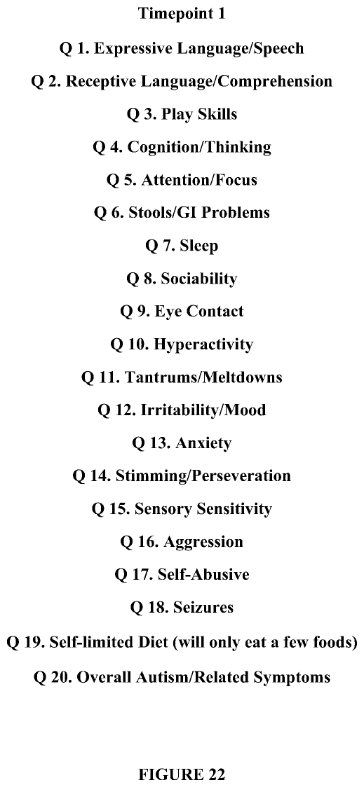

Results are shown in FIGS. 4-21.

### Example 3

### Tracking of the Microbiome in an ASD Cohort

Presented here is longitudinal tracking of the microbiome and its genetic material of the study cohort of Example 2 to show the restoration of gut microbial diversity after dietary supplements, medical food, or biotherapeutic treatment.

The dietary supplement may include and is not limited to probiotics, prebiotics, post-biotics, digestive enzymes, vitamins, minerals, natural extracts, botanical, and other food ingredients. Alpha and beta diversity of whole genome metagenomic sequencing on the microbiome and its genetic material was assessed at longitudinal timepoints and demonstrated statistical differences in gut microbial populations between ASD and NT controls. When treated with custom dietary supplements, the gut microbial diversity of the ASD cohort increased to levels that are not statistically different from the NT control group. Using surveys such as PGIA, SRS-2, GSRS, DSM, CARS, and SCARED, improvement of paired samples can be tracked. Gastrointestinal symptom improvement was shown from timepoint 1 to timepoint 2 over a 2-6 month period of taking a custom dietary supplement. Improvement over multiple categories of symptoms known to be associated with ASD through the PGIA survey was observed. Further analysis of the microbial populations between NT (healthy controls) vs. ASD show specific organisms that may be implicated, diagnostic, prognostic of the disorder. Analysis of the genetic material can further determine the genetic potential of biochemical pathways that are different between the healthy controls and the ASD.

### Results

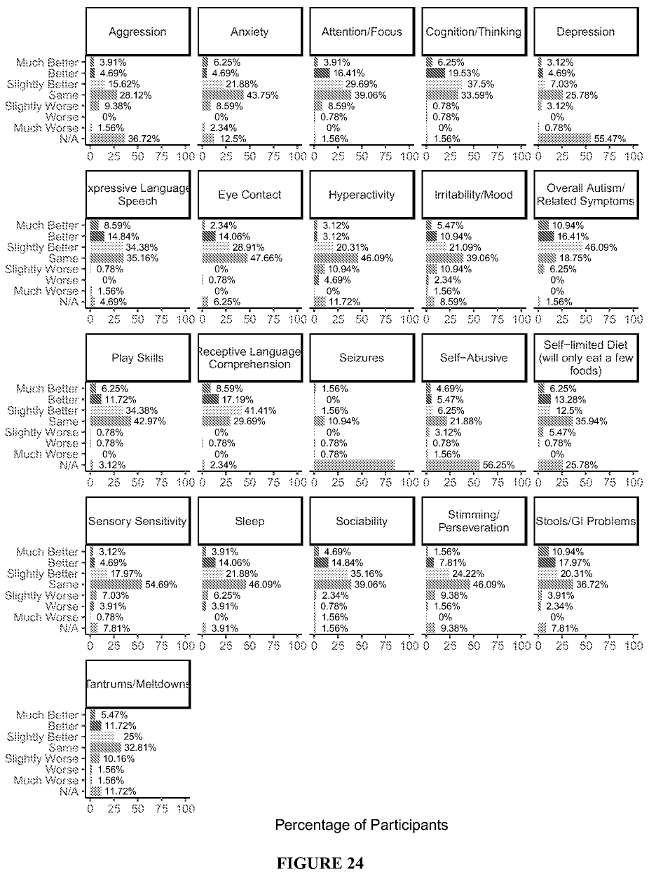

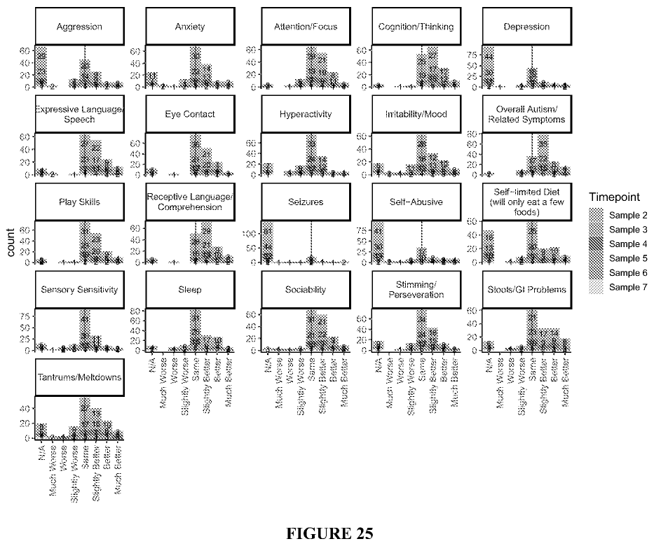

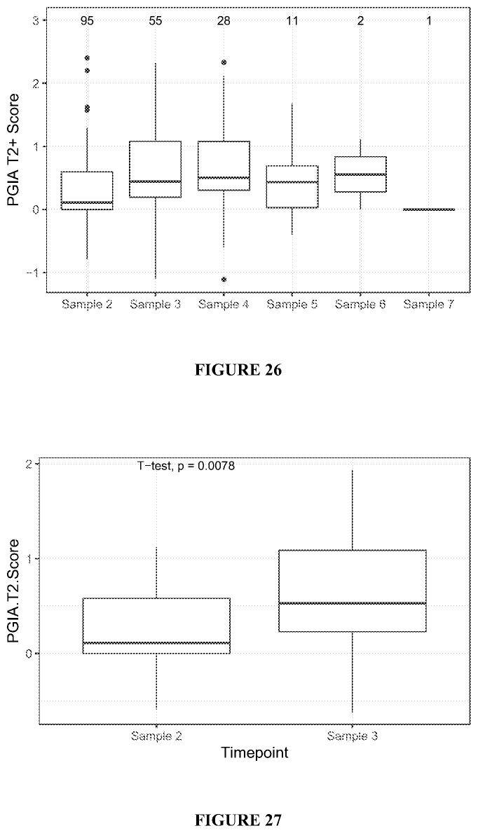

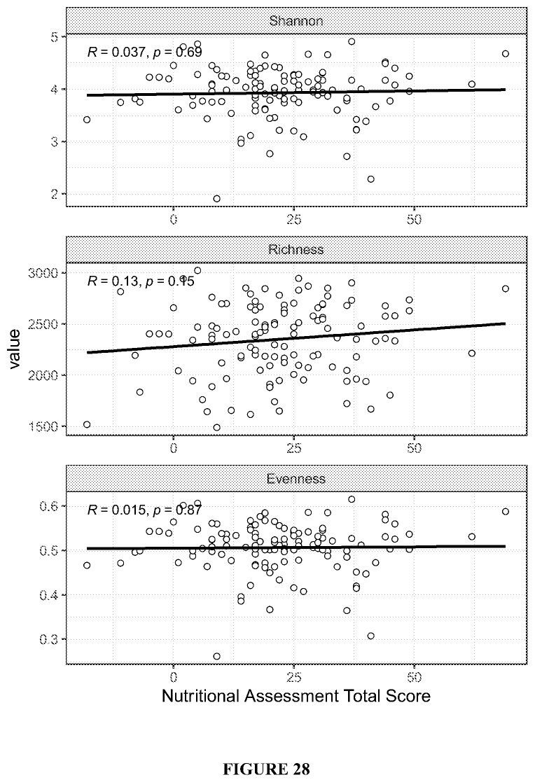

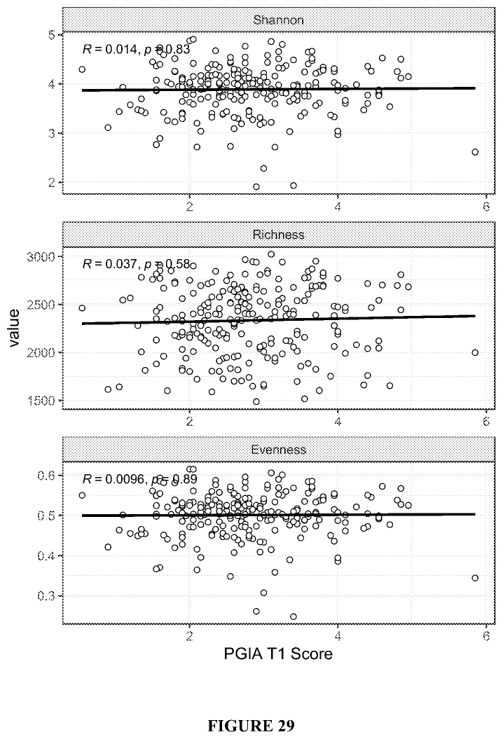

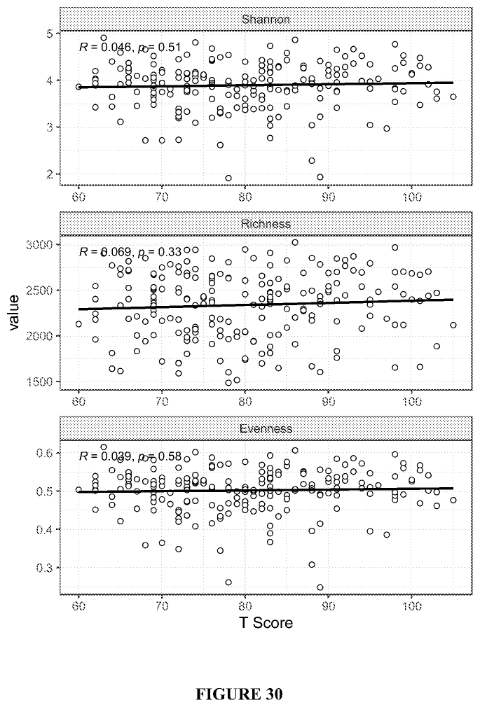

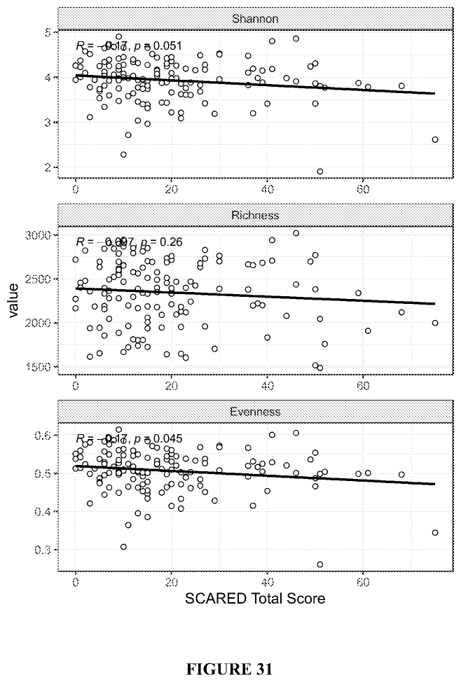

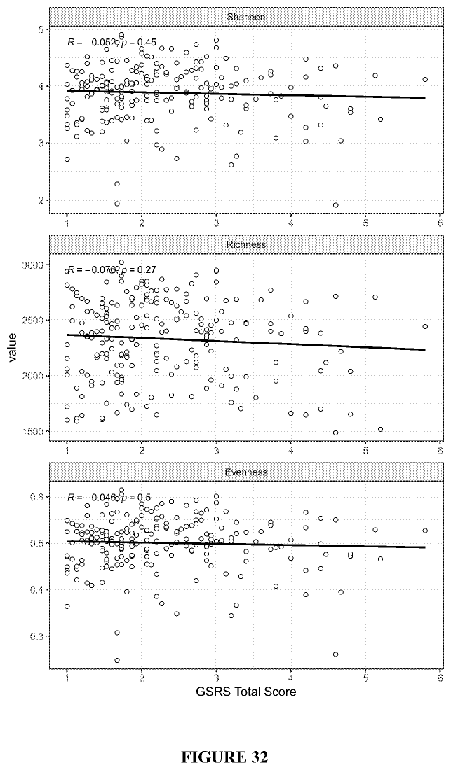

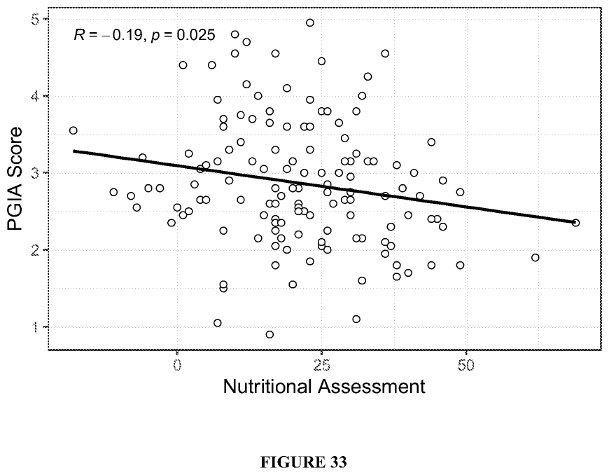

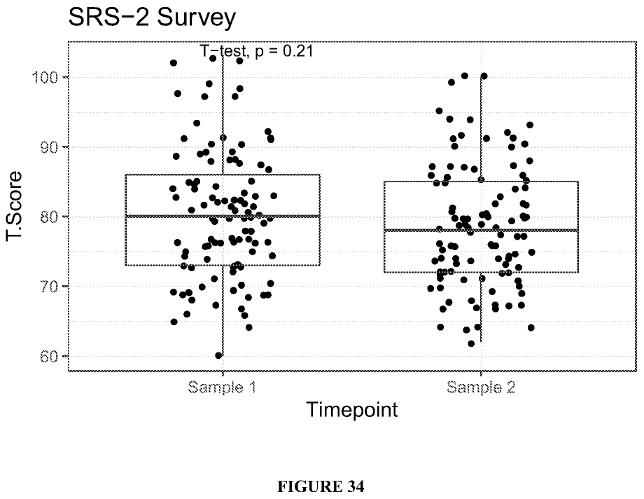

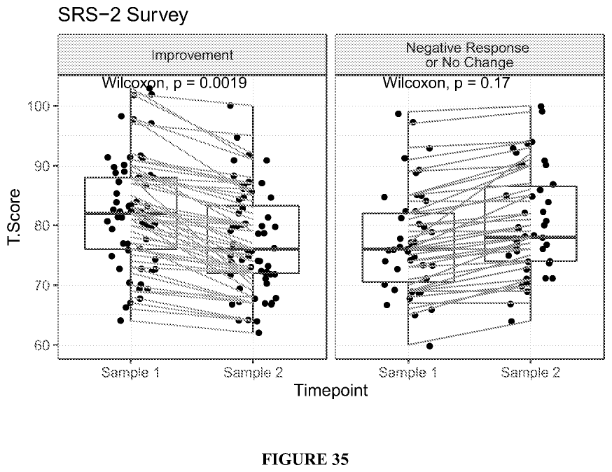

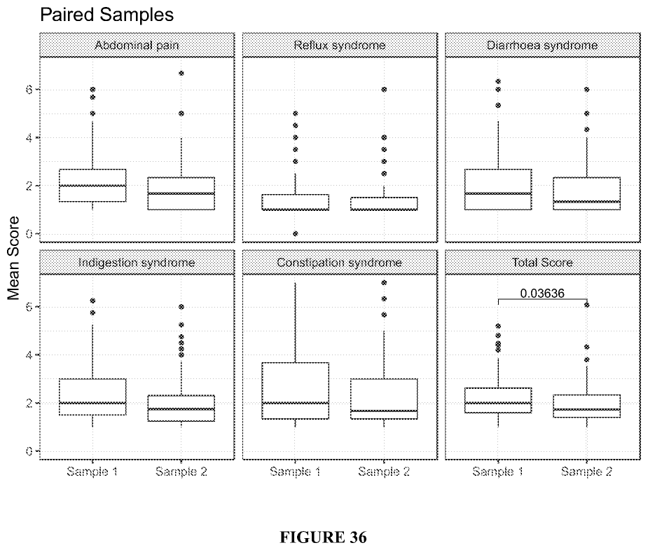

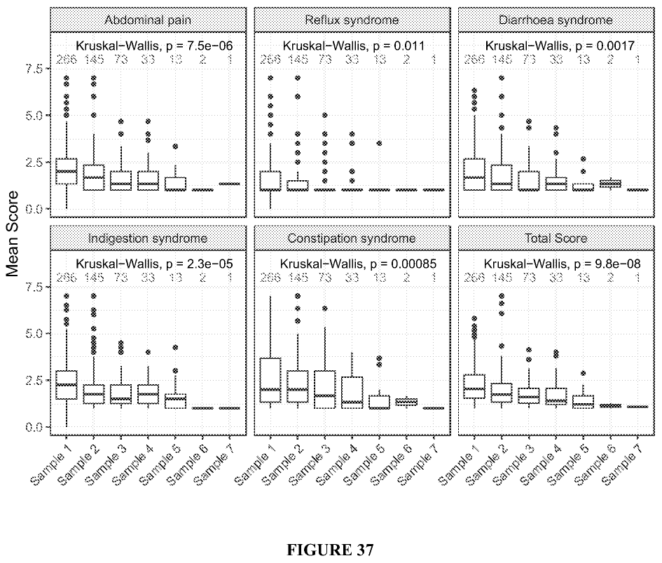

Results are shown in FIGS. 24-37.

### Example 4

### Comparison of Microbiome Profile Between Healthy Pilot Whales and Sick Pilot Whales

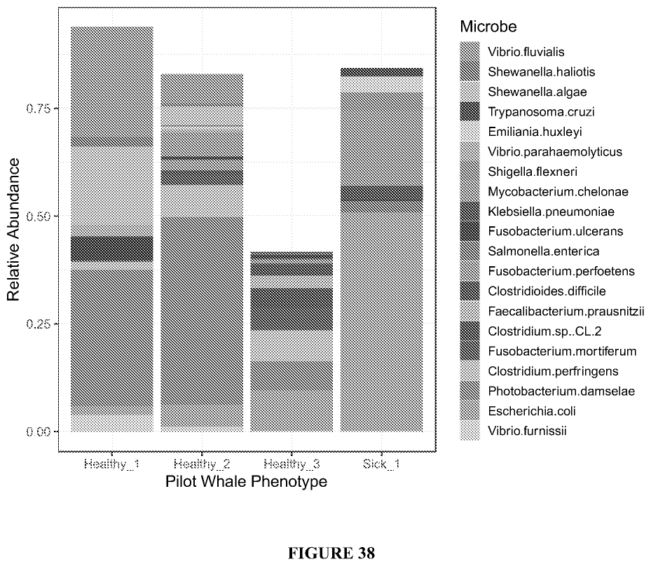

Presented here is a comparison study of the microbiome profiles of healthy and sick pilot whales. A direct comparison of the microbiome profiles between healthy whales and a sick whale is shown in FIG. 38. The sick whale was reported to have ulcerative colitis in the survey. The profile for this whale was represented with high abundance levels of pathogens including 47% E. coli, 16% Mycobacterium chelonae, Klebsiella pneumoniae, Salmonella and Shigella compared to the control whales (FIG. 38).

The healthy whales were mostly represented with Photobacterium damselae subsp. Damselae, found above 40% and is considered a primary pathogen of several species of wild fish causing wound infections and hemorrhagic. Clostridium perfringens is associated with intestinal diseases in humans and animals. Vibrio fluvialis is a Gram-negative microbe commonly found in coastal environments. Mycobacterium chelonae has been found to be associated with tumor-like lesions in sturgeon and associated with mycobacteriosis in aquatic animals.

TABLE 6
Pilot Whale Study Population

| Whale | kit ID | SG numbers |
|---|---|---|
| Healthy_1 | 24004463 | SG014270 |
| Healthy_2 | 24004455 | SG014282 |
| Healthy_3 | 24003851 | SG012081 |
| Ulcerative | 24004446 | SG013764 |
| colitis | | |

Although the invention has been described with reference to the above example, it will be understood that modifications and variations are encompassed within the spirit and scope of the invention. Accordingly, the invention is limited only by the following claims.

1. A method for determining a microbial signature in a microbiome comprising:
analyzing microbiomes from a healthy control population, wherein analyzing comprises performing metagenomics analysis and classifying microbial taxa and/or biological pathways;
analyzing microbiomes from a population having a disease or disorder, wherein analyzing comprises performing metagenomics analysis and classifying microbial taxa and/or biological pathways; and
identifying a microbial signature by comparing the analysis of microbiomes from the healthy control population to the analysis of the microbiomes from the population having the disease or disorder, wherein identifying comprises determining a difference in presence or relative abundances of i) microbial taxa, and/or ii) biological pathways, between the microbiomes from the healthy control population and the microbiomes from the population having the disease or disorder, thereby determining a microbial signature in a microbiome.

2. The method of claim 1, wherein determining the difference in presence or relative abundances of microbial taxa comprises determining an increase and/or decrease in relative abundances of microbial taxa in the microbiomes from the population having the disease or disorder as compared to the relative abundances of microbial taxa in the microbiomes from the healthy control population.

3. The method of claim 1, wherein determining the difference in presence or relative abundances of microbial taxa comprises determining relative abundances of different microbial populations.

4. The method of claim 1, wherein the microbial populations are selected from bacteria, parasites, archaea, fungi, viruses and phage.

5. The method of claim 1, wherein determining the difference in presence or relative abundances of biological pathways comprises determining an increase and/or decrease in relative abundances of biological pathways in the microbiomes from the population having the disease or disorder as compared to the relative abundances of microbial taxa in the microbiomes from the healthy control population.

6. The method of claim 1, wherein the disease or disorder is selected from the group consisting of irritable bowel syndrome (IBS), autism spectrum disorder (ASD), arthritis, obesity, dysbiosis, Crohn's disease, mood disorder, chronic fatigue, infection, necrosis, inflammation, autoimmune, hemorrhage, weight loss, metabolic disorder, diabetes 1 or 2, rheumatoid arthritis, cancer, and cardiovascular disorder.

7. The method of claim 6, wherein the disease or disorder is IBS.

8. The method of claim 6, wherein the disease or disorder is ASD.

9. The method of claim 8, wherein the ASD is Asperger's syndrome, pervasive developmental disorder-not otherwise specified (PDD-NOS) or autistic disorder.

10. The method of claim 1, wherein determining the difference in presence or relative abundances of biological pathways comprises analyzing a coding or non-coding region of a microbial genome.

11. The method of claim 10, wherein determining the difference in presence or relative abundances of biological pathways comprises analyzing the coding region of the microbial genome.

12. The method of claim 11, wherein the coding region is a gene.

13. The method of claim 12, wherein the gene is associated with a biological pathway.

14. The method of claim 1, further comprising correlating the microbial signature with meta data from a standardized survey.

15-35. (canceled)

36. A method of diagnosing, prognosing and/or determining risk or severity of irritable bowel syndrome (IBS) in a subject comprising:
obtaining a sample comprising a microbiome from the subject;
performing metagenomics analysis on the sample;
classifying microbial taxa and/or biological pathways in the microbiome;
determining a microbial signature of the microbiome indicative of IBS based on the classifying; and
diagnosing, prognosing and/or determining risk or severity of IBS in the subject based on the microbial signature, thereby diagnosing, prognosing and/or determining risk or severity IBS in the subject.

37. The method of claim 36, wherein IBS is IBS-C, IBS-D, IBS-M or IBS-U.

38. (canceled)

39. The method of claim 36, further comprising assigning an overall microbiome score to the subject.

40. The method of claim 36, wherein the sample is a gut or fecal sample.

41. The method of claim 36, wherein metagenomics analysis comprises whole genome sequencing.

42-66. (canceled)

67. A method of diagnosing, prognosing and/or determining risk or severity of autism spectrum disorder (ASD) in a subject comprising:
obtaining a sample comprising a microbiome from the subject;
performing metagenomics analysis on the sample;
classifying microbial taxa and/or biological pathways in the microbiome;
determining a microbial signature of the microbiome indicative of ASD based on the classifying; and
diagnosing, prognosing and/or determining risk or severity of ASD in the subject based on the microbial signature, thereby diagnosing, prognosing and/or determining risk or severity ASD in the subject.

68-97. (canceled)
